(19) United States
(12) Patent Application Publication
(10) Pub. No.: US 2026/0165733 A1
(43) Pub. Date: Jun. 18, 2026

(54) INTEGRATED DEVICE AND SYSTEM FOR EPIDURAL INJECTION

(71) Applicant: BEJING SIGHTNOVO MEDICAL TECHNOLOGY CO., LTD, Beijing (CN)

(72) Inventors: Chaoran XIA, Beijing (CN); Chan ZHAO, Beijing (CN); Chuan LI, Beijing (CN)

(21) Appl. No.: 19/125,198

(22) PCT Filed: Nov. 10, 2023
(86) PCT No.: PCT/CN2023/130955

§ 371 Date: Apr. 28, 2025

(30) Foreign Application Priority Data
Nov. 11, 2022 (WO) PCT/CN2022/131455

(51) Int. Cl. A61B 17/34; A61M 5/00; A61M 5/20; A61M 5/31; A61M 5/32; A61M 25/00; A61M 25/06

(52) U.S. Cl. CPC A61B 17/3401; A61M 5/2033; A61M 5/3129; A61M 5/3286; A61M 5/329; A61M 25/065; A61M 2005/006; A61M 2005/3128; A61M 2005/3131; A61M 2025/0007; A61M 2202/048; A61M 2210/1003

(57) ABSTRACT

A medical puncturing device and a system. The medical puncturing device includes a syringe barrel (1), a gasket seal (3), a push shaft (2), an elastic element (12) and a catheter (16). The medical puncturing device enables injection, access, expansion, and/or device implantation in an apparent or potential tissue void, cavity, or vessel (e.g., an epidural space), and is especially useful for achieving precise control of puncturing depth and needle placement, as well as steady injection and injection of a defined volume (e.g., for epidural anesthesia).

### CROSS-REFERENCE TO RELATED APPLICATIONS

This application claims priority to International Patent Application No. PCT/CN2022/131455, entitled “DEVICE AND SYSTEM FOR EPIDURAL INJECTION,” having an international filing date of Nov. 11, 2022. The disclosure and content of the above-referenced application is incorporated herein by reference in its entirety for all purposes.

### FIELD

The present disclosure in some aspects relates to the field of medical device and apparatus, and specifically a device, kit, assembly, or system for epidural injection.

### BACKGROUND

In existing methods of treatment involving epidural anesthesia, a regular syringe is typically used to inject a local anesthetic into the epidural space. When performing the puncture, the punctuation position and depth of a needle of the syringe needs to be manually controlled, and a medical personnel has to rely on his or her experience to determine if the needle has entered the epidural space. However, the depth and structure of various tissues around and in the epidural space of different patients usually vary from each other and the determination of needle depth by the medical personnel may not be accurate and reliable. As a result, the precise placement of the needle relative to the epidural cannot be guaranteed. Furthermore, it is hard for medical personnel to visualize whether the needle tip has reached the targeted injection site (e.g., an epidural space). Improved devices and methods for medical penetration such as injection into the epidural space are needed. The present disclosure addressed these and other needs.

### SUMMARY

In some embodiments, provided herein is an injection system. In any of the preceding embodiments, the injection system can comprise a syringe barrel extending from a proximal end to a distal end. In any of the preceding embodiments, the injection system can comprise a first hollow needle extending from a proximal end to a distal end comprising an end opening, wherein the needle distal end is connected to the distal end of the syringe barrel. In any of the preceding embodiments, the hollow needle can comprise markings on an outside wall of the hollow needle to indicate the insertion depth of the hollow needle. In any of the preceding embodiments, the injection system can comprise a floating seal, wherein the floating seal is positioned inside the syringe barrel, forms a lumen between the floating seal and the distal end of the syringe barrel, and comprises a hollow channel configured to align with the first hollow needle. In any of the preceding embodiments, the injection system can comprise a push shaft with a hollow channel extending from a proximal end to a distal end, wherein the distal end of the push shaft is proximal to and in contact with the floating seal, and the hollow channel of the push shaft is configured to align with the hollow channel of the floating seal and the first hollow needle to form a central hollow channel extending from the proximal end of the push shaft to the distal opening of the first hollow needle. In any of the preceding embodiments, the injection system can comprise a proximal seal at the proximal end of the central hollow channel. In any of the preceding embodiments, the injection system can comprise an actuation unit comprising an actuation member and an energy storage member, wherein the actuation member is configured to elastically engage the push shaft via the energy storage member.

In any of the preceding embodiments, the energy storage member can comprise a spring between the floating seal and the push shaft. In any of the preceding embodiments, the spring can elastically engage a proximal portion of the floating seal and a distal portion of the push shaft. In any of the preceding embodiments, the distal end of the push shaft can elastically engage the floating seal. In any of the preceding embodiments, the push shaft can be configured to directly or indirectly couple with a pathway triggering component (e.g., an alarm and/or a light sensor) to signal upon the needle reaching a predetermined depth or a predetermined position.

In any of the preceding embodiments, the injection system can further comprise a piercing unit comprising a proximal needle and a needle guiding structure. In any of the preceding embodiments, the proximal needle can be configured to advance distally in a hollow needle guiding channel inside the needle guiding structure to pierce the proximal seal. In any of the preceding embodiments, the needle guiding structure can comprise a side port configured to align with a catheter guiding channel, such that a catheter in the catheter guiding channel is configured to advance through the side port into the hollow needle guiding channel inside the needle guiding structure, through the pierced proximal seal, and into the central hollow channel.

In any of the preceding embodiments, the injection system can further comprise an injection syringe comprising a second hollow needle configured to insert into the central hollow channel for injection of a composition. In any of the preceding embodiments, the first hollow needle can be configured to be placed in an epidural space. In any of the preceding embodiments, the injection system can further comprise a catheter configured to be inserted through the first hollow needle into an epidural space. In any of the preceding embodiments, the second hollow needle can be configured to withdraw from the central hollow channel following an injection of a composition. In any of the preceding embodiments, the catheter can be configured to withdraw from an epidural space through the first hollow needle.

In some embodiments, disclosed herein is a method for epidural injection. In any of the preceding embodiments, the method can comprise using any injection system disclosed herein to inject a composition into an epidural space. In some embodiments, disclosed herein is a method for epidural injection. In any of the preceding embodiments, the method can comprise using any injection system disclosed herein to place a catheter into an epidural space, and injecting a composition into the epidural space through the catheter.

In some embodiments, provided herein is a device comprising a syringe barrel extending from a proximal end to a distal end, wherein the distal end of the syringe barrel comprises a needle base configured to be coupled to a needle comprising a needle lumen, and wherein the needle base comprises a passageway configured to fluidically communicate with the needle lumen. In any of the preceding embodiments, the device can comprise a gasket seal that forms a fluid-tight seal with an inner wall of the syringe barrel, wherein a flowable composition lumen is formed between the gasket seal and the distal end of the syringe barrel, wherein the gasket seal comprises a through hole along an axis of the syringe barrel, and wherein the through hole is configured to align with the passageway in the needle base. In any of the preceding embodiments, the device can comprise a push shaft extending from a proximal end to a distal end, wherein the distal end of the push shaft engages the gasket seal, and the push shaft comprises a central channel comprising a distal end that aligns with the through hole of the gasket seal and a valve that aligns with a proximal end of the central channel. In any of the preceding embodiments, the device can comprise an elastic element configured to actuate the push shaft such that the gasket seal is moved distally along the axis of the syringe barrel. In some embodiments, the central channel and the passageway in the needle base are connected when the gasket seal is moved distally. In any of the preceding embodiments, the device can comprise a catheter configured to be inserted through the valve and into the central channel. In any of the preceding embodiments, the needle can be an epidural needle. In any of the preceding embodiments, the needle is a Tuohy epidural needle, a Hustead epidural needle, a Crawford epidural needle, or a Weiss epidural needle. In any of the preceding embodiments, the needle can be between about 17 G and about 22 G (iso-9626) in gauge size. In any of the preceding embodiments, the needle can be 17-18 G (iso-9626) in gauge size. In any of the preceding embodiments, the needle can be 19-20 G (iso-9626) in gauge size. In any of the preceding embodiments, the needle can be between about 2.5 and about 6 inches in length. In any of the preceding embodiments, the needle can be between about 3 and about 3.5 inches in length. In any of the preceding embodiments, the needle can comprise a straight distal tip. In any of the preceding embodiments, the needle can comprise a curved distal tip. In any of the preceding embodiments, the needle can comprise a distal tip comprising a blunt bevel. In any of the preceding embodiments, the needle can comprise a side port. In any of the preceding embodiments, the side port of the needle can be configured to allow injection of an anesthetic agent through the needle lumen.

In any of the preceding embodiments, the gasket seal can be configured to allow distal advancement of the catheter through the through hole. In any of the preceding embodiments, the gasket seal can be configured to allow proximal retraction of the catheter through the through hole. In any of the preceding embodiments, the through hole can be configured to close in the absence of the catheter or the central channel inserted in the gasket seal. In any of the preceding embodiments, the through hole can be configured to allow the catheter or the central channel to pass through the gasket seal.

In any of the preceding embodiments, the central channel can pass through the through hole of the gasket seal. In any of the preceding embodiments, the distal end of the central channel can be flush with the distal end of the through hole. In any of the preceding embodiments, the distal end of the central channel can be flush with the proximal end of the through hole. In any of the preceding embodiments, the push shaft can further comprise a central chamber in fluidic communication with the central channel. In any of the preceding embodiments, the central chamber can be at least partially between the valve and the proximal end of the central channel. In any of the preceding embodiments, the valve can be configured to allow one-way passage of the catheter. In any of the preceding embodiments, the push shaft can comprise a catheter guiding channel. In any of the preceding embodiments, the valve can align with a distal end of the catheter guiding channel. In any of the preceding embodiments, the valve can be at the distal end of the catheter guiding channel. In any of the preceding embodiments, the push shaft can comprise a side port configured to allow passage of the catheter through the side port into the catheter guiding channel. In any of the preceding embodiments, the push shaft can comprise a locking mechanism configured to maintain a position of the gasket seal in the syringe barrel and a compressed or expanded state of the elastic element.

In any of the preceding embodiments, the elastic element can comprise a spring, a rubber band, a bungee cord, a memory foam, an air bag, or a combination thereof. In any of the preceding embodiments, a distal end of the elastic element can engage a portion of the push shaft or the gasket seal and a proximal end of the elastic element can engage a portion of the syringe barrel. In any of the preceding embodiments, a distal end of the elastic element (e.g., spring) can engage a portion of the push shaft and a proximal end of the elastic element (e.g., spring) can engage a structure of or in the syringe barrel, such as a baffle of or in the syringe barrel. In any of the preceding embodiments, the elastic element can be configured to be compressed. In any of the preceding embodiments, the decompression of the compressed elastic element can exert a force to actuate the push shaft or the gasket seal such that the gasket seal is moved distally along the axis of the syringe barrel. In any of the preceding embodiments, a distal end of the elastic element can engage a portion of the syringe barrel and a proximal end of the elastic element can engage a portion of the push shaft or the gasket seal. In any of the preceding embodiments, the elastic element can be configured to be expanded. In any of the preceding embodiments, the collapse of the expanded elastic element can exert a force to actuate the push shaft or the gasket seal such that the gasket seal is moved distally along the axis of the syringe barrel.

In any of the preceding embodiments, the catheter can be between about 19 G and about 20 G (iso-9626) in gauge size. In any of the preceding embodiments, the device can comprise a housing accommodating at least a portion of the syringe barrel, at least a portion of the push shaft, and at least a portion of the catheter. In any of the preceding embodiments, the device can comprise a catheter storage mechanism and a catheter actuation mechanism. In any of the preceding embodiments, the housing can define the outside boundaries of the device, with all components contained within the housing. In any of the preceding embodiments, at least a portion of the catheter can be inserted through the valve. In any of the preceding embodiments, at least a portion of the catheter can be inserted into the central channel. In any of the preceding embodiments, at least a portion of the catheter can be passed through the through hole of the gasket seal. In any of the preceding embodiments, at least a portion of the catheter can be passed through the passageway of the needle base. In any of the preceding embodiments, at least a portion of the catheter can be inserted into the needle lumen. In any of the preceding embodiments, the catheter can comprise a distal end configured to form a coil. In any of the preceding embodiments, the catheter can comprise markings on an outside wall of the catheter to indicate the insertion depth and/or the insertion location of the distal end of the catheter.

In any of the preceding embodiments, the device can comprise (a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen; (b) a gasket seal that forms a fluid-tight seal with an inner wall of the syringe barrel, wherein the gasket seal abuts the distal end of the syringe barrel, wherein the gasket seal comprises a through hole along an axis of the syringe barrel, and wherein the through hole aligns with the passageway in the needle base; (c) a push shaft extending from a proximal end to a distal end, wherein the distal end of the push shaft engages the gasket seal, and the push shaft comprises a central channel comprising a distal end that aligns with the through hole of the gasket seal and a valve that aligns with a proximal end of the central channel; (d) a spring, wherein a distal end of the spring engages a portion of the push shaft, and a proximal end of the spring engages a structure (e.g., a baffle) in or of the syringe barrel, wherein the spring is configured to be compressed, and decompression of the compressed spring actuates the push shaft or the gasket seal such that the gasket seal is moved distally along the axis of the syringe barrel; (e) a catheter configured to be inserted through the valve and into the central channel, and (f) a housing accommodating at least a portion of the syringe barrel, at least a portion of the push shaft, and at least a portion of the catheter.

In any of the preceding embodiments, the device can comprise (a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen; (b) a gasket seal that forms a fluid-tight seal with an inner wall of the syringe barrel, wherein a flowable composition lumen is formed between the gasket seal and the distal end of the syringe barrel, wherein the gasket seal comprises a through hole along an axis of the syringe barrel, and wherein the through hole aligns with the passageway in the needle base; (c) a push shaft extending from a proximal end to a distal end, wherein the distal end of the push shaft engages the gasket seal, and the push shaft comprises a central channel comprising a distal end that aligns with the through hole of the gasket seal and a valve that aligns with a proximal end of the central channel; (d) a spring, wherein a distal end of the spring engages a portion of the push shaft, and a proximal end of the spring engages a structure (e.g., a baffle) in or of the syringe barrel, wherein the spring is compressed, and decompression of the compressed spring actuates the push shaft or the gasket seal such that the gasket seal is moved distally along the axis of the syringe barrel; (e) a catheter configured to be inserted through the valve and into the central channel, and (f) a housing accommodating at least a portion of the syringe barrel, at least a portion of the push shaft, and at least a portion of the catheter. In any of the preceding embodiments, the flowable composition lumen can contain a gas. In any of the preceding embodiments, the gas can be air. In any of the preceding embodiments, the flowable composition lumen can contain no liquid.

In any of the preceding embodiments, the device can comprise (a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen; (b) a gasket seal that forms a fluid-tight seal with an inner wall of the syringe barrel, wherein the gasket seal comprises a through hole along an axis of the syringe barrel, and wherein the through hole aligns with the passageway in the needle base; (c) a push shaft extending from a proximal end to a distal end, wherein the distal end of the push shaft engages the gasket seal, and the push shaft comprises a central channel comprising a distal end that aligns with the through hole of the gasket seal and a valve that aligns with a proximal end of the central channel; (d) a spring, wherein a distal end of the spring engages a portion of the push shaft, and a proximal end of the spring engages a structure (e.g., a baffle) in or of the syringe barrel, wherein the spring exerts a force on the push shaft or the gasket seal such that the gasket seal abuts the distal end of the syringe barrel, and wherein the central channel and the passageway in the needle base are configured to be connected; (e) a catheter configured to be inserted through the valve and into the central channel, and (f) a housing accommodating at least a portion of the syringe barrel, at least a portion of the push shaft, and at least a portion of the catheter.

In any of the preceding embodiments, the device can comprise (a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen; (b) a gasket seal that forms a fluid-tight seal with an inner wall of the syringe barrel, wherein the gasket seal comprises a through hole along an axis of the syringe barrel, and wherein the through hole aligns with the passageway in the needle base; (c) a push shaft extending from a proximal end to a distal end, wherein the distal end of the push shaft engages the gasket seal, and the push shaft comprises a central channel comprising a distal end that aligns with the through hole of the gasket seal, a valve that aligns with a proximal end of the central channel, and a catheter guiding channel, wherein the valve aligns with a distal end of the catheter guiding channel; (d) a spring, wherein a distal end of the spring engages a portion of the push shaft, and a proximal end of the spring engages a structure (e.g., a baffle) in or of the syringe barrel, wherein the spring exerts a force on the push shaft or gasket seal such that the gasket seal abuts the distal end of the syringe barrel, and wherein the central channel and the passageway in the needle base are configured to be connected; (e) a catheter, wherein a portion of the catheter is in the catheter guiding channel; and (f) a housing accommodating at least a portion of the syringe barrel, at least a portion of the push shaft, and at least a portion of the catheter. In any of the preceding embodiments, the distal end of catheter can be in the catheter guiding channel of the push shaft. In any of the preceding embodiments, the distal end of catheter can be inserted through the valve of the push shaft. In any of the preceding embodiments, the distal end of catheter can be in the central channel of the push shaft or the through hole of the gasket seal. In any of the preceding embodiments, the distal end of catheter can be in the passageway of the needle base. In any of the preceding embodiments, the distal end of catheter can be in the needle lumen of the needle.

In some embodiments, provided herein is a method for accessing an epidural space of a subject. In any of the preceding embodiments, the method can comprise a step of (a) coupling a needle to a device, wherein the needle comprises a needle tip, a needle lumen and a needle hub, and the device comprises (i) a syringe barrel extending from a proximal end to a distal end, wherein the distal end of the syringe barrel comprises a needle base configured to be coupled to the needle, and wherein the needle base comprises a passageway configured to fluidically communicate with the needle lumen; (ii) a gasket seal that forms a fluid-tight seal with an inner wall of the syringe barrel, wherein the gasket seal abuts the distal end of the syringe barrel, wherein the gasket seal comprises a through hole along an axis of the syringe barrel, and wherein the through hole aligns with the passageway in the needle base; (iii) a push shaft extending from a proximal end to a distal end, wherein the distal end of the push shaft engages the gasket seal, and the push shaft comprises a central channel comprising a distal end that aligns with the through hole of the gasket seal and a valve that aligns with a proximal end of the central channel; (iv) a spring, wherein a distal end of the spring engages a portion of the push shaft, and a proximal end of the spring engages a structure (e.g., a baffle) in or of the syringe barrel, wherein the spring is configured to be compressed; and (v) a catheter configured to be inserted through the valve and into the central channel. In any of the preceding embodiments, the method can comprise a step of (b) proximally actuating the push shaft, thereby compressing the spring and forming a flowable composition lumen between the gasket seal and the distal end of the syringe barrel, wherein the flowable composition lumen contains a gas. In any of the preceding embodiments, the method can comprise a step of (c) locking the push shaft to maintain a compressed state of the spring and maintain a position of the gasket seal in the syringe barrel. In any of the preceding embodiments, the method can comprise a step of (d) advancing the needle tip in the subject towards a location in the ligamenta flava of the subject, without a user holding the push shaft to maintain the compressed state of the spring. In any of the preceding embodiments, the method can comprise a step of (e) unlocking the push shaft when the needle tip is in the ligamenta flava. In any of the preceding embodiments, the method can comprise a step of (f) advancing the needle tip through the ligamenta flava into an epidural space of the subject, thereby allowing the compressed spring to decompress such that the gasket seal is moved distally to connect the central channel and the passageway in the needle base. In any of the preceding embodiments, the method can comprise a step of (g) injecting an anesthetic agent through a side port of the needle into the needle lumen, thereby injecting anesthetic agent into the epidural space of the subject. In any of the preceding embodiments, a second needle can be inserted through the side port of the needle to reach the subarachnoid space of the subject. In any of the preceding embodiments, a second needle can be inserted through the side port of the needle into the needle lumen, and then through the distal opening of the needle, thereby placing a distal end of the second needle in the subarachnoid space of the subject. In any of the preceding embodiments, an anesthetic agent through can be injected through the second needle into the subarachnoid space. In any of the preceding embodiments, the second needle can be withdrawn from the subarachnoid space following the injection of the anesthetic agent. In any of the preceding embodiments, the method can comprise a step of (h) inserting the catheter through the valve, the central channel, the through hole, the passageway, and the needle lumen, thereby placing a distal portion of the catheter in the epidural space. In any of the preceding embodiments, the method can comprise a step of (i) uncoupling the needle from the needle base to remove the catheter from the device, while the distal portion of the catheter remains in the epidural space. In any of the preceding embodiments, the proximal portion of the catheter can be fixed on the outside of the subject (e.g. on the skin). In any of the preceding embodiments, the method can comprise a step of using the catheter for epidural anesthesia by continuous infusion or intermittent bolus, optionally by manually delivered intermittent bolus (MIB) and programmable intermittent bolus (PIB). In any of the preceding embodiments, the method can comprise a step of using the catheter for intraoperative epidural anesthesia and/or postoperative analgesia.

In some embodiments, disclosed herein is a multifunctional anesthesia device for intraspinal anesthesia. In some embodiments, the multifunctional anesthesia device used for intraspinal anesthesia comprises a puncture needle, a first injection component (e.g., one comprising a syringe, a plunger, a pull rod or push shaft, and/or a one-way valve), a pressurizing component (e.g., one comprising a spring, a main rod, and/or a pull rod or push shaft), a catheter placement mechanism (e.g., one comprising a gear set, a roller, a clamp, and/or a catheter), a second injection component (e.g., one comprising an injection needle and/or a fixing seat), and a casing/housing. In some embodiments, the materials of one or more of the components of the device include but are not limited to medical polymer materials, aluminum alloy, stainless steel, etc. In some embodiments, the outer surface of the casing or housing can be designed with patterns or a frosted effect for easy grip during operation. In some embodiments, the device can comprise one or more transmission components, for instance, including gears and rollers. In some embodiments, the catheter can be placed inside the injection syringe through any one or more of the transmission component(s). In some embodiments, turning the gears clockwise can push the catheter through the injection syringe and puncture needle into the epidural space.

In some embodiments, before using a device disclosed herein, a patient can be instructed to assume a proper position, a suitable puncture point is selected, the skin of the selected point is disinfected, and a sterile drape is placed, and then the puncture point is locally infiltrated with anesthesia. In some embodiments, during use, an operator can grip the outer shell and align the epidural needle with the puncture point. In some embodiments, the puncture path is from the skin, to a subcutaneous tissue, to a supraspinous ligamentum, to an interspinous ligamentum, or a ligamentum flavum, and to an epidural space. In some embodiments, when the needle pierces the ligamentum flavum, the spring in the device senses the disappearance of resistance and automatically releases pressure, indicating that the needle has reached the epidural space. In some embodiments, a subarachnoid injection needle can be inserted (e.g., through a side port) to perform a subarachnoid anesthesia. After that, the subarachnoid injection needle can be withdrawn and a knob can be rotated (e.g., clockwise) to advance a catheter through the puncture needle into the epidural space to the appropriate length. Then an operator can grab the outer shell/casing/housing and remove the puncture needle, secure the catheter, and the patient can turn over and lie flat. If necessary, epidural anesthesia can be administered through the catheter.

In some embodiments, a device disclosed herein is an integrated device where a syringe, an anesthesia puncture needle, and an epidural anesthesia catheter are consolidated into one device, reducing operational steps and saving surgical time. In some embodiments, a device disclosed herein is configured to perform an automatic pressure release function: when the anesthesia puncture needle passes through the ligamentum flavum, the device can sense the disappearance of pushing resistance and automatically release pressure through a spring, indicating that the needle has been punctured into place, reducing the risks associated with human judgment. In some embodiments, a device disclosed herein is configured to achieve continuous drug delivery: through the epidural anesthesia catheter and drug deliver pump, continuous and repeated drug administration can be achieved, thereby achieving postoperative pain relief.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1E show schematic diagrams of the different stages of operating an exemplary medical puncturing device, for example, during epidural anesthesia and/or injection into epidural space 14. FIG. 1F show steps of operating an exemplary medical puncturing device without a contacting member (e.g., 1b shown in FIGS. 1A-1E), where a distal seal (e.g., 8 shown in FIGS. 1A-1E) may directly contact a tissue.

FIGS. 2A-2G show schematic diagrams of the different stages of operating an exemplary medical puncturing device, for example, during epidural anesthesia and/or injection into epidural space 14. FIG. 2F shows steps of operating an exemplary medical puncturing device without a contacting member (e.g., 1b shown in FIGS. 2A-2E), where a distal seal (e.g., 8 shown in FIGS. 2A-2E) may directly contact a tissue. FIG. 2G shows steps of operating an exemplary medical puncturing device comprising an additional actuation member 2′ engaging floating seal 3 via another spring 4′, whereas actuation member 2 engages floating seal 3 via spring 4.

FIGS. 3A-3F are partial structure diagrams of exemplary medical puncturing devices comprising floating seal 3 and one or more needle body openings (6b or 6b1, 6b2, and/or 6b3) and needle distal opening 6a.

FIGS. 4A-4C are partial structure diagrams of exemplary medical puncturing devices comprising floating seal 3 and needle body opening 6b.

FIGS. 5A-5F are partial structure diagrams of exemplary medical puncturing devices comprising floating seals 3a and 3b and one or more needle body openings (6b or 6b1 and/or 6b2).

FIG. 6 shows a partial structure diagram of an exemplary medical puncturing device comprising a through angled guiding groove 3a and one-way valve 9.

FIG. 7 shows a partial structure diagram of an exemplary medical puncturing device comprising a through angled guiding groove 3a and one-way valve 9.

FIG. 8 shows a partial structure diagram of an exemplary medical puncturing device comprising a non-through angled guiding groove 3a.

FIG. 9 shows a partial structure diagram of an exemplary medical puncturing device comprising an angled guiding needle hole 6c and one-way valve 9.

FIG. 10 shows a partial structure diagram of an exemplary medical puncturing device comprising an angled guiding needle hole 6c and needle hole plug 10.

FIG. 11 shows a schematic diagrams of implanting catheter 11 into epidural space 14 using an exemplary medical apparatus assembly comprising a central guiding groove 2c.

Reference numerals and exemplary corresponding structures are provided below for illustration only, and should not be considered limiting: 1—syringe barrel; 1a—axial stopper; 1b—circular contacting element; 2—pressing element; 2c—central guiding groove; 3—floating seal; 3a—angled guiding groove; 4—elastic sheath; 5—spring; 6—hollow puncture needle; 6a—needle distal opening; 6b—needle body opening; 6c—angled guiding needle hole; 7—flowable composition lumen; 8—distal seal; 9—one—way valve; 10—needle hole plug; 11—catheter; 12—auxiliary guiding needle; 13—denser tissue (e.g., ligamentum flavum); 14—potential or apparent tissue void, cavity, or vessel (e.g., epidural space).

FIGS. 12A-12C show schematic diagrams of the different stages of operating an exemplary medical puncturing device.

FIG. 13 shows an example of epidural injection, e.g., for epidural anesthesia.

FIGS. 14A-14B show schematic diagrams of an exemplary injection system. FIG. 14A shows the outside view of the exemplary injection system. FIG. 14B shows the inside view of the exemplary injection system.

FIG. 15 shows schematic diagrams of different stages of operating an exemplary injection system.

Reference numerals and exemplary corresponding structures are provided below for illustration only, for instance, with reference to FIGS. 14A-14B and FIG. 15, and should not be considered limiting: 1—syringe barrel; 2—push shaft; 2a—control knob; 3—floating seal; 5—spring; 6—hollow needle structure; 6′—piercing unit (to pierce the proximal seal); 8a—proximal seal; 11—catheter; 12a—stabilizing structure; 12b—catheter guiding channel; 40—main housing; 41—handle; 42—rotation wheel of catheter insertion unit; 43—needle guiding structure; 44—side port; 50—drug injection syringe.

FIGS. 16A-16B show schematic diagrams of an exemplary integrated devices. FIG. 16A shows the outside view of the exemplary integrated device. FIG. 16B shows the inside view of the exemplary integrated injection system.

FIG. 17 shows a structure diagram of an exemplary integrated device.

Reference numerals and exemplary corresponding structures are provided below for illustration only, for instance, with reference to FIGS. 16 and 17, and should not be considered limiting: 1—syringe barrel; 2—push shaft; 3—gasket seal; 4—needle base; 5—needle; 6—needle lumen; 7—passageway; 8—flowable composition lumen; 9—through hole; 10—central channel; 11—valve; 12—elastic element; 13—catheter guiding channel; 14—needle hub; 15—needle side port; 16—catheter; 17—stabilizing structure; 18—distal tip; 19—side port; 20—central chamber; 21—; 40—housing; 41—handle; 42—rotation wheel of catheter insertion unit; 43—catheter storage mechanism.

FIG. 18 shows schematic diagrams of different stages of operating an exemplary integrated device.

### DETAILED DESCRIPTION

Below is a detailed description of some embodiments of the present disclosure. It should be understood that the specific implementations described herein are meant to illustrate and explain the embodiments of the present disclosure, and should not be considered limiting.

It should be noted that, when not in conflict, the embodiments of the present disclosure and the features of the embodiments may be combined in any suitable manner.

In some embodiments, the positional descriptions of “front,” “back,” “forward,” “backward,” “distal,” and “proximal,” etc. are based on the perspective of an operator of the medical puncturing device or medical apparatus assembly. That is, when the operator is using the medical puncturing device or medical apparatus assembly, the direction pointing away and relatively far from the operator is the forward direction, and the direction pointing toward and relatively close to the operator is the backward direction.

As used herein, the words “proximal” and “distal” refer to the direction closer to and away from, respectively, an operator (e.g., surgeon, physician, nurse, technician, etc.) who would insert the medical device into the patient, with the tip-end (distal end) of the device inserted inside a patient's body first. Thus, for example, the end of a needle (e.g., microneedle) described herein first inserted inside the patient's body would be the distal end, while the opposite end of the needle (e.g., the end of the medical device being manipulated by the operator) would be the proximal end of the needle.

As used herein, the singular forms “a,” “an,” and “the” include plural referents unless the context clearly dictates otherwise. For example, “a” or “an” means “at least one” or “one or more.” Likewise, the term “a member” is intended to mean a single member or a combination of members, “a material” is intended to mean one or more materials, or a combination thereof.

The term “about” or “approximately” as used herein refers to the usual error range for the respective value readily known to the skilled person in this technical field. Reference to “about” a value or parameter herein includes (and describes) embodiments that are directed to that value or parameter per se. For example, “about” can mean within 1 or more than 1 standard deviation, per the practice in the relevant field. Alternatively, “about” can mean a range of up to 20%, up to 10%, up to 5%, or up to 1% of a given value.

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the terms “puncture member”, and “puncturing member” are used interchangeably to refer to an article configured to pierce tissue layers and deliver a substance to a target tissue layer, for example, a needle or a microneedle.

As used herein, the terms “medicament container”, and “medicament containment chamber” are used interchangeably to refer to an article (e.g., a syringe) configured to contain a volume of a substance, for example, a medicament or drug.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The medical puncture device and medical appliance assembly of the present invention can be used for the penetration, expansion and/or injection of cavities such as the epidural space, as well as the implantation of drugs, catheters or other medical devices into the epidural space. At present, injection of drugs and/or medical devices into the epidural space has been broadly used in a variety of treatments, such as epidural anesthesia. Currently, epidural anesthesia is conducted via injection of one or more local anesthetics into the epidural space to block the conduction function of some spinal nerves and temporarily paralyze or anesthetize the innervated area of those spinal nerves, and this method is usually called epidural space block anesthesia. To perform epidural space block anesthesia, anesthesiologists need to perform an epidural puncture, during which the puncture needle sequentially passes through the skin, subcutaneous, supraspinous ligament, interspinous ligament, ligamentum flavum, and reaches the epidural space. When the puncture needle tip reaches the epidural space, anesthesiologist can usually feel a pressure drop at the needle tip. Following this feeling of pressure drop, the anesthesiologists then place an epidural catheter into the epidural space, withdraw the puncture needle, leave one side of the catheter in epidural space and secure the catheter, so that a drug can be administered in batches through the catheter and provide continuous anesthesia. Alternatively, the continuous anesthesia can be administered using an infusion pump coupled to the catheter to achieve the desired postoperative analgesic effect.

In some instances, targeted injection of a therapeutic agent into the epidural space is desirable. In such instances, however, the complicated structure of the tissues around and in the epidural space and the difficulty to visualize them during the injection often result in significant challenges to placing a needle at a target location using known devices and methods, especially as they pertain to placing the distal end of the needle at the desired depth. Therefore, during epidural anesthesia, one key issue is how to accurately determine the distal end of the puncture needle has reached the epidural space.

Existing anesthesia instruments and methods basically rely on doctor's experience and feel to determine whether the distal end of the puncture needle has entered the epidural space, which has a relatively low degree of reliability, requires high operating skills of the doctor, and cannot guarantee the precision and safety of each puncture position. In addition, current methods are usually complicated, have high material and human cost, have low efficiency, tend to cause anesthesia failure or postoperative complications, frequently bring pain to the patient, and sometimes even cause damage to the spinal cord or nerves.

For example, one commonly used main instrument for epidural anesthesia is a low-resistance syringe. Because of the relatively lower pressure in the epidural space, the pressure at the distal opening of the low resistance syringe can be felt by medical personnel via the resistance felt by the medical personnel during injection, therefore the medical personnel can tell the position of the distal opening of the puncture needle. When the distal opening of the needle is advanced into the subcutaneous tissue to reach the dense ligament tissue, the needle distal opening is blocked by the dense tissues, and the medical personnel can feel a large pressure and resistance when the push rod is advanced distally. When the medical personnel feel a sudden pressure drop or reduction of resistance, they can preliminary tell the needle distal opening has reached the epidural space. However, in order to confirm this, the medical personnel must pull and push the push rod and see if any fluid is drawn into the syringe. If during the pull and push of the push rod, resistance of doing so remains small, and no fluid is drawn into the syringe, then the needle distal opening has reached the desired injection site. If some cerebrospinal fluid is drawn into the syringe, it means that the puncture needle has entered the subarachnoid space and must be re-positioned. Because a catheter needs to be placed into the epidural space after the epidural puncture, if the epidural punctuation fails, or if the needle distal opening is not at the correct position or depth, the catheter being put in can enter the subarachnoid space or blood vessels, which may further lead to total spinal cord block or systemic poisoning, severe hypotension, loss of consciousness and respiratory arrest. If no appropriate actions are taken promptly, this may even be followed with cardiac arrest. In some instance, the catheter has a long leading stroke and is inconvenient to operate. As result, the precision and safety of drug injections into the epidural space with low-resistance syringe highly rely on the experience and operation skill of the medical personnel, and are hard to control or improve.

Therefore, providing an epidural anesthesia device and method with precise puncture position, straightforward and rapid operation and high anesthesia success rate to provide epidural anesthesia has great clinical benefits and practical significance.

Aiming at the existing technical problems and shortcomings, to reduce the surgical risk and operation difficulty of epidural anesthesia, a special epidural anesthesia device with pressure sensing indication function is developed, which is an integrated structure with integrated design. The pressure difference between the ligament and the epidural space is judged by the pressure sensing component, so as to accurately locate the placement of the epidural needle. The epidural catheter control mechanism allows the built-in catheter to be easily pushed out and along the needle into the epidural space. The device has the advantages of automatic and precise positioning of the epidural needle tip in the epidural space, simple catheter placement, simplified operation process, high surgical success rate and high efficiency.

In some embodiments, the epidural anesthesia puncture device disclosed herein comprises a puncture needle, syringe components (syringe, push rod, sealing piston, sealing gasket), elastic components (spring, sleeve), knob, catheter mechanism (gripper, runner, fixed shaft), guide rod assembly (guide rod, needle), and operating handle. In some embodiments, the needle can be used to puncture the ligamentum flavum and feel obvious resistance, then the knob compresses the spring, the liquid/gas in the syringe is pre-pressurized, and the puncture is continued slowly until the sealing piston moves forward, and the liquid/gas in the syringe barrel is released, indicating the puncture needle tip has entered the epidural space, and the operator can stop the needle advancement at this point. When the sealing piston reaches the bottom of the syringe, the guide rod can be pushed and the needle will pierce the sealing gasket to form a channel. If necessary, the syringe can be connected to a 25 G lumbar puncture needle to administer anesthesia to the subarachnoid space, and then the syringe and lumbar puncture needle can be withdrawn. In some embodiments, a catheter can be delivered forward, and whether it is in place can be judged by observing the scale of the catheter. After the catheter is in place, an operator can release the gripper by pressing the button, fix the catheter with one hand, and remove the epidural anesthesia puncture device with the other hand, in order to complete the catheter placement. Afterwards, according to clinical needs, multiple doses of anesthesia or continuous anesthesia can be performed. In some embodiments, the epidural anesthesia puncture device disclosed herein can also be used to administer a drug into an intraspinal space in an intraspinal anesthesia. In some embodiments, the epidural anesthesia puncture device disclosed herein can also be used to administer a drug into an intrathecal space in an intrathecal anesthesia. Exemplary devices and steps are shown in FIGS. 14A-14B and FIG. 15.

In order to achieve one or more of the purposes mentioned above, the present disclosure provides in a medical puncturing device comprising: a syringe barrel, wherein the syringe barrel comprises a distal closed end and a proximal open end; an actuation unit (e.g., an elastic movement unit) comprising an actuation member (e.g., pressing element) and a floating seal, wherein the floating seal is positioned inside the syringe barrel and can elastically engage with the actuation member (e.g., pressing element); a hollow puncture needle attached to the actuation member (e.g., pressing element), wherein the hollow puncture needle comprises a needle distal opening and a needle body opening, and wherein the needle body opening is proximal to the floating seal (the needle distal opening can be proximal to the floating seal, e.g., the entire length of the needle is proximal to the floating seal, or alternatively, the needle can be through the floating seal such that the needle distal opening is distal to the floating seal); and a flowable composition lumen (e.g., for a fluid or gel), wherein the flowable composition lumen is formed by the syringe barrel distal closed end, a syringe barrel lumen wall (e.g., a portion of the syringe barrel), and the floating seal.

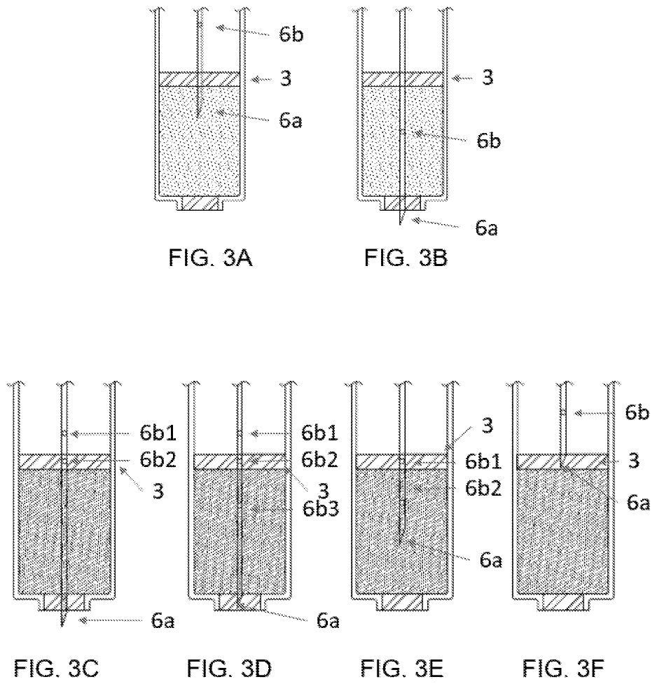

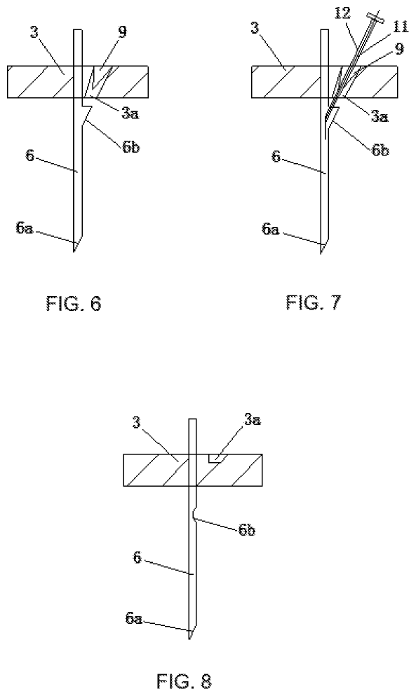

In some embodiments, the medical puncturing device is configured such that the hollow puncture needle can be moved forward by pressing the actuation member (e.g., pressing element). In some embodiments, the hollow puncture needle sequentially pierces the floating seal and the syringe barrel distal closed end, thus connecting the flowable composition lumen, the needle body opening, and the needle distal opening. In some embodiments, the hollow puncture needle is pre-inserted into the floating seal. For example, the needle distal opening can be in the floating seal and blocked by the floating seal, and the needle can be advanced through the flowable composition lumen to pierce the syringe barrel distal closed end. In some embodiments, the hollow puncture needle is pre-inserted through the floating seal. For example, the needle distal opening can be in the flowable composition lumen, while the needle body opening is proximal to the floating seal or in the floating seal (e.g., the needle body opening can be blocked by the floating seal as shown in FIG. 3E), and then the needle can be advanced to pierce the syringe barrel distal closed end. In some embodiments, the hollow puncture needle is pre-inserted through the floating seal and in or through the syringe barrel distal closed end. For example, the needle distal opening can be in a distal seal at the syringe barrel distal closed end (e.g., the needle distal opening can be blocked by the distal seal) or distal to the distal seal and/or the syringe barrel distal closed end, while the needle body opening is proximal to the floating seal (e.g., as shown in FIGS. 3D, 6b1), in the floating seal (e.g., the needle body opening can be blocked by the floating seal as shown in FIGS. 3D, 6b2), or in the flowable composition lumen (e.g., as shown in FIGS. 3D, 6b3), and then the needle can be advanced through the syringe barrel distal closed end and exposing the needle distal opening for puncturing a tissue.

Optionally, the medical puncturing device comprises a state wherein the flowable composition lumen, the needle body opening, and the needle distal opening are in fluidic communication. For example, in a fluidic communication state, the needle body opening can be proximal to the floating seal, while the needle distal opening is distal to the floating seal and in the flowable composition lumen. In the fluidic communication state, the needle and/or the floating seal can be moved. For example, the floating seal can be moved under the elastic resilience between the floating seal and the actuation member (e.g., pressing element) such as that the floating seal seals or blocks the needle body opening, thereby preventing or terminating discharge of the flowable composition (such as a gel) from the needle body opening and/or from the needle distal opening.

Optionally, in the fluidic communication state, the floating seal can seal the needle body opening when it moves forward and contacts the syringe barrel distal closed end, thereby preventing or terminating discharge of the flowable composition (such as a gel) from the needle body opening and/or from the needle distal opening.

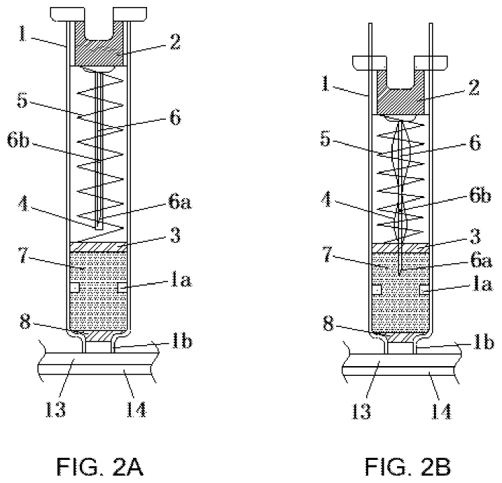

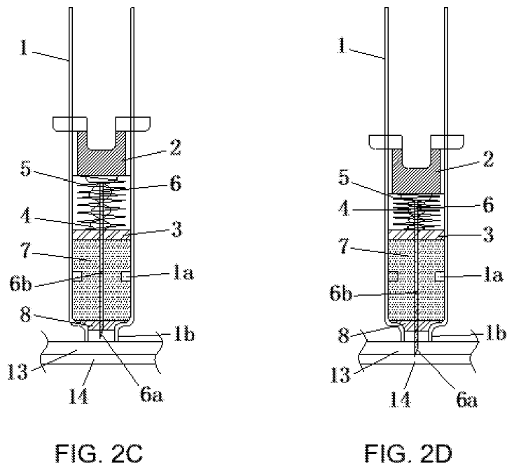

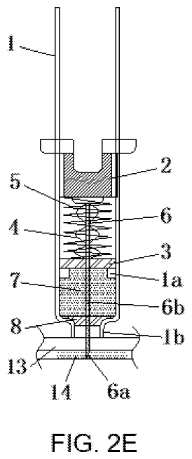

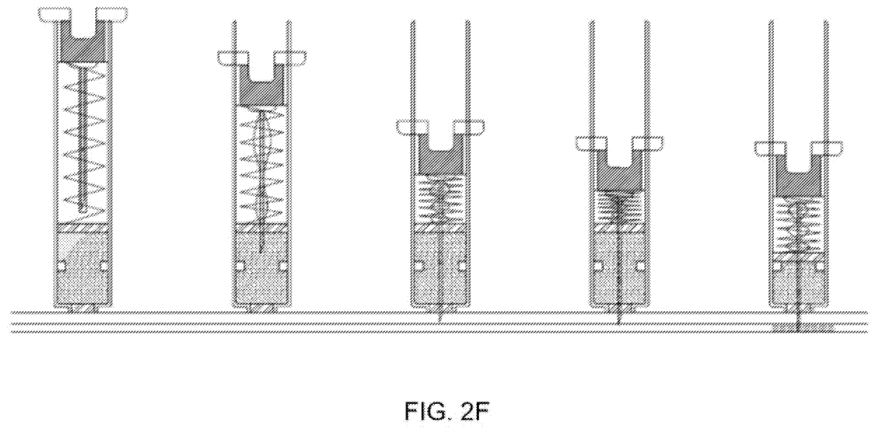

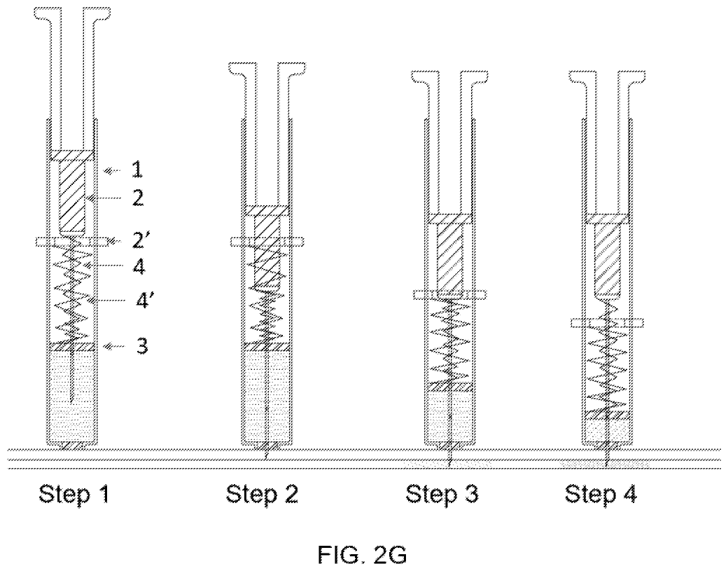

Optionally, a s stopper such as an axial stopper can be provided inside the syringe lumen, distal to the floating seal. In some embodiments, the stopper can be used to limit the forward movement of the floating seal. In some embodiments, the medical puncturing device comprises a fluidic communication state, wherein the flowable composition lumen is connected to the needle body opening and the needle distal opening. When the medical puncturing device is in the fluidic communication state, the needle body opening can be at the distal end of the stopper (e.g., as shown in FIG. 2D), and the floating seal can move forward due to the elastic engagement with the actuation member (e.g., pressing element).

Optionally, the medical puncturing device comprises a manual control element, which is attached to the floating seal and is extended outside of the syringe barrel.

Optionally, the medical puncturing device comprises a pre-puncture state after the hollow puncture needle pierces the syringe barrel distal closed end, a surface tissue puncture state, and a fluidic communication state after the puncture. In the pre-puncture state, the surface tissue puncture state, and the fluidic communication state, the length range of the hollow puncture needle extended outside of the syringe barrel distal closed end can correspond to a pre-puncture length range, a surface tissue puncture length range, and a fluidic communication length range, respectively, wherein: when the length of the of the hollow puncture needle extended outside of the syringe barrel distal closed end is within the pre-puncture length range, the needle body opening remains above the flowable composition lumen (e.g., the needle body opening can be proximal to and within the floating seal); and/or when the length of the of the hollow puncture needle extended outside of the syringe barrel distal closed end is within the surface tissue puncture length range, at least part of the needle body opening is connected to the flowable composition lumen; and/or when the length of the of the hollow puncture needle extended outside of the syringe barrel distal closed end is within the fluidic communication length range, the needle body opening is positioned within the flowable composition lumen.

Optionally, an axially extended circular contacting element is formed at the syringe barrel distal closed end, wherein the difference between the upper and lower limits of the pre-puncture length range equals to the axial length of the circular contacting element.

Optionally, the elastic movement unit comprises a elastic sheath covering the outside of the hollow puncture needle. When the needle body opening is proximal to the floating seal, the elastic sheath can seal the needle body opening. In some embodiments, when the flowable composition is a gel, it may not be necessary to seal the needle body opening when it is proximal to the floating seal.

Optionally, the medical puncturing device comprises a catheter guiding structure which is used to thread the catheter into a cavity (e.g., a needle body passageway connected to the needle distal opening and/or the needle body opening) of the hollow puncture needle.

Optionally, the catheter guiding structure comprises an angled guiding groove which is formed on the floating seal and extends towards the hollow puncture needle in an angle.

Optionally, the angled guiding groove is set to be through the floating seal in the front and back direction. In some embodiments, the catheter guiding structure further comprises a one-way valve which is embedded in the angled guiding groove and can be opened and closed, and/or a guiding groove plug inserted in the angled guiding groove.

Optionally, the angled guiding groove is set to be on the upper surface of the floating seal and is a non-through groove.

Optionally, the needle body opening is formed as an angled opening which opens obliquely backwards.

Optionally, the catheter guiding structure comprises an angled guiding needle hole formed on the body wall of the hollow puncture needle and opens obliquely backwards. In some embodiments, the medical puncturing device comprises a fluidic communication state wherein the flowable composition lumen is in connection with the needle body opening and the needle distal opening. In the fluidic communication state, the angled guiding needle hole is positioned proximal to the floating seal.

Optionally, the catheter guiding structure further comprises a one-way valve which is embedded in the angled guiding needle hole and can be opened and closed, or a guiding groove plug inserted in the angled guiding needle hole.

Optionally, the catheter guiding structure comprises a puncturable central guiding groove that is formed on the center of the proximal surface of the actuation member (e.g., pressing element). In some embodiments, a needle proximal opening is formed on the hollow puncture needle and the needle proximal opening is set to axially align with the central guiding groove.

Optionally, the medical puncturing device comprises a puncture control module and a fluid storage module that are independently manufactured and formed, wherein: the puncture control module comprises a first syringe unit and the elastic movement unit and the hollow puncture needle provided inside the first syringe unit; the fluid storage module comprises a second syringe unit, the flowable composition lumen formed inside the barrel of the second syringe unit, and a module packaging component which is removably packaged to the proximal end of the second syringe unit; and a removable connection structure is formed between the first syringe unit and the second syringe unit.

In a second aspect, the present disclosure provides a medical apparatus assembly. In some embodiments, the medical apparatus assembly comprises a catheter and the medical puncturing device comprising a catheter guiding structure.

Optionally, the medical apparatus assembly further comprises a hollow auxiliary guiding needle which is matched to use with the catheter guiding structure. In some embodiments, when the auxiliary guiding needle is connected to the catheter guiding structure, the catheter can sequentially go through the needle body passageway of the auxiliary guiding needle and the catheter guiding structure and be threaded into the needle body passageway of the hollow puncture needle.

In some embodiments, when using the medical puncturing device of the present disclosure, a user can first apply pressure to the actuation member (e.g., pressing element) to drive the hollow puncture needle sequentially through the floating seal and the syringe barrel distal closed end. When the needle distal opening of the hollow puncture needle reaches apparent or potential tissue gaps, cavity systems, and vessels, the needle body opening has already been positioned in the flowable composition lumen, and the floating seal has already formed an elastic engagement with the actuation member (e.g., pressing element). In some embodiments, the fluid pressure in the flowable composition lumen can be made higher than the pressure inside the an apparent or potential tissue void, cavity, or vessel.

At this time, the fluid inside the flowable composition lumen can flow into the an apparent or potential tissue void, cavity, or vessel through the needle body opening and the needle distal opening. During the injection process, just by maintaining the position of the actuation member (e.g., pressing element), under the action of the elastic engagement between the floating seal and the actuation member (e.g., pressing element), the fluid inside the flowable composition lumen can flow into the needle body opening (and then through the needle body passageway and out of the needle distal opening), thereby achieving injection, penetration, and/or expansion of the an apparent or potential tissue void, cavity, or vessel. Additionally, the medical apparatus assembly as describe in the present disclosure can achieve implantation of catheter and other medical device through the medical puncturing device, e.g., through a catheter guiding structure and a cavity of the needle described herein.

In some embodiments, before the hollow puncture needle pierces into an apparent or potential tissue void, cavity, or vessel, the external pressure on the needle distal opening is higher than the fluid pressure in the flowable composition lumen, thus fluid cannot flow out of the needle distal opening. Thus, by observing whether the floating seal moves forward due to the elastic engagement with the actuation member (e.g., pressing element), it is possible to determine whether the hollow puncture needle has already pierced into an apparent or potential tissue void, cavity, or vessel, thereby reminding the operator of the current punctuation depth to ensure accurate puncture. Since the injection is controlled by fluid pressure changes in the flowable composition lumen, the injection process does not require an operator to manually apply thrust or force during the injection process, thus fluctuations in the flow speed can be prevented and stable injection can be achieved.

Other features and advantages of the present disclosure will be described in the detailed description below.

Some embodiments of the present disclosure will be described with reference to the several views of the accompanying drawings.

### II. Systems and Devices

In some embodiments, disclosed herein is a multifunctional anesthesia device for intraspinal anesthesia, characterized in that it includes: a puncture needle; a position determination component connected to the puncture needle to sense when the puncture needle reaches a predetermined position; a pathway triggering component to establish a pathway for the introduction of anesthetic fluid after the puncture needle reaches the predetermined position; an insertion and withdrawal component to inject anesthetic fluid into the predetermined position; a catheter insertion device to push the catheter through the pathway into the epidural space (also set in a specific position, which can also be included in the dependent claims; wherein the catheter insertion device, the insertion and withdrawal component, the pathway triggering component, and the position determination component are all located within a housing. In some embodiments, the multifunctional anesthesia device for intraspinal anesthesia is characterized in that the catheter introduction device is configured to push the catheter through the introducer pathway into the epidural space. In some embodiments, the multifunctional anesthesia device for intrathecal anesthesia is characterized in that the position determination component comprises one or more elastic components such as springs. In some embodiments, the multifunctional anesthesia device for intrathecal anesthesia is characterized in that the position determination component is configured to sense the arrival of the puncture needle at the predetermined position. In some embodiments, the outer shell of the multi-functional anesthesia device for intrathecal anesthesia is shaped to snap-connect two half shells.

In some embodiments, described herein are systems and devices to assist in the insertion of a puncture member, for example, a needle or microneedle into the epidural space, and/or assist in injecting a medicament into a target ocular tissue. In some embodiments, described herein are systems and devices for controlling the insertion depth of a puncture member, such as, for example, a needle, into the epidural space to deliver anesthetics and/or therapeutic agents. In some embodiments, described herein are systems and devices for introducing an implant into a tissue, such as an apparent or potential tissue void, cavity, or vessel. In some embodiments, the device disclosed herein can also be used to administer a drug into an intraspinal space in an intraspinal anesthesia. In some embodiments, the epidural anesthesia puncture device disclosed herein can also be used to administer a drug into an intrathecal space in an intrathecal anesthesia.

In some embodiments, provided herein is a system comprising a syringe barrel comprising a proximal end and a distal end; a floating seal in the syringe barrel; a needle base proximal to the floating seal (e.g., the needle base is closer to an operator while the floating seal is closer to a subject), and the floating seal and the needle base are configured to elastically engage each other. In some embodiments, the system further comprises a needle comprising a needle proximal end and a needle distal end, and the needle proximal end engages the needle base. In any of the embodiments herein, the needle proximal end can be fixed to the needle base or releasably attached to (e.g., inserted in) the needle base. In any of the embodiments herein, the needle can comprise: (i) a needle distal opening, (ii) a needle body opening between the needle proximal end and the needle distal end, and (iii) a needle body passageway connecting the needle distal opening and the needle body opening. In any of the embodiments herein, the needle body opening can be proximal to the needle distal opening. In any of the embodiments herein, the needle base can be configured to advance the needle distally toward the floating seal (e.g., when the needle distal end is proximal to the floating seal), through the floating seal (e.g., when the needle distal end has entered or pierced into the floating seal), and/or through the distal end of the syringe barrel.

In some embodiments, a device disclosed herein comprises or is configured to be coupled to a medicament container containing a medicament, such as a solution, a liquid, a suspension, a gel, or the like. The medicament container can be formed at least in part by the syringe barrel.

Unlike certain existing devices in which a needle is coupled to a distal end of a medicament container (e.g., the needle is at the distal end of a syringe, for example, as described in U.S. Pat. Nos. 9,180,047, 9,539,139, 9,572,800, 9,636,253, 9,636,332, 9,770,361, 9,937,075, 10,555,833, and 10,517,756, which are incorporated herein by reference for all purposes), in some embodiments, the present disclosure utilizes a needle that is coupled to an actuation member inside a syringe barrel. In some embodiments, a need disclosed herein is at least partially inside the syringe barrel. In some embodiments, prior to use, the needle neither is exposed at the distal end of the syringe barrel nor directly engages the distal end of the syringe barrel.

In some embodiments, a device disclosed herein comprises an energy storage member (e.g., one or more springs) configured to engage the needle base and the floating seal. In some embodiments, a distal end portion of the energy storage member is configured to be disposed within the syringe barrel and directly or indirectly engage the floating seal. In some embodiments, the energy storage member is configured to produce a force on a proximal end portion of the floating seal. In some embodiments, the force is sufficient to move the floating seal within the syringe barrel to convey at least a portion of a substance from the medicament container (e.g., a flowable composition lumen) via the needle when a distal tip of the needle is disposed within an apparent or potential tissue void, cavity, or vessel. Furthermore, the force is insufficient to move the floating seal within the syringe barrel when the distal tip of the needle is disposed within a tissue adjacent to (e.g., above or below) the apparent or potential tissue void, cavity, or vessel. In some embodiments, the apparent or potential tissue void, cavity, or vessel has a first density and the adjacent tissue has a second density, higher than the first density. In some embodiments, the apparent or potential tissue void, cavity, or vessel produces a first backpressure and the adjacent tissue produces a second backpressure, higher than the first backpressure.

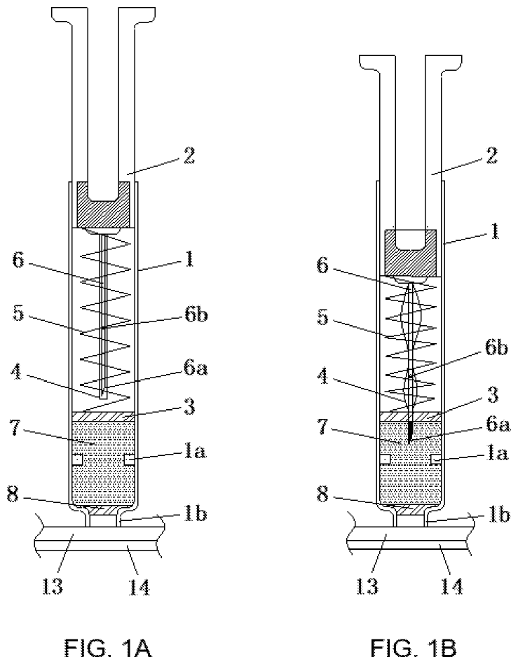

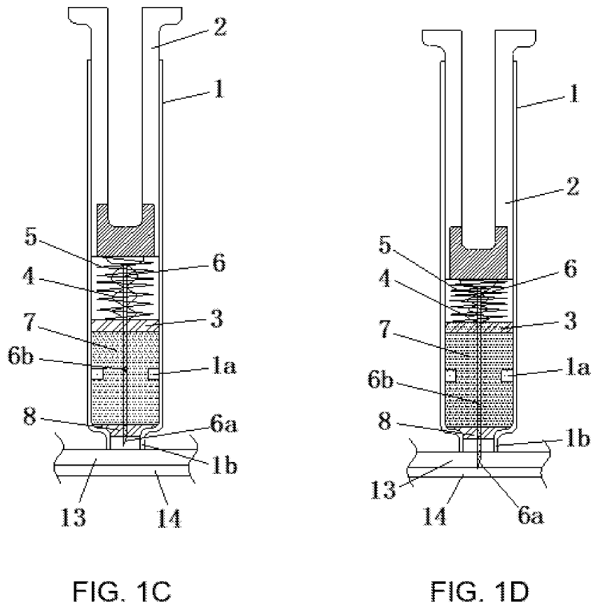

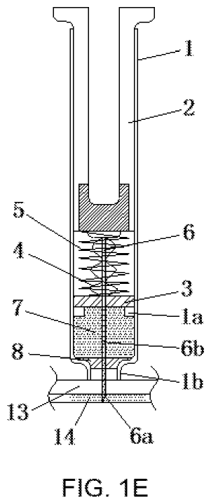

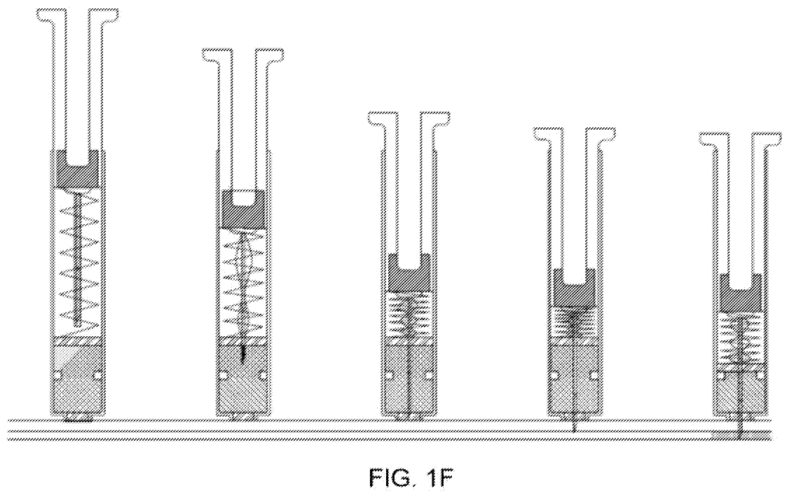

In some embodiments, a device disclosed herein comprises an energy storage member (e.g., one or more springs, e.g., spring 5 in FIGS. 1A-1E or FIG. 12,) configured to exert a force on a floating seal directly (e.g., as shown in FIGS. 1A-1E) or indirectly (e.g., as shown in FIG. 12, via a piston rod 15). In some embodiments, the energy storage member is configured to exert a force on the floating seal that is between the pressure in a first tissue and the pressure in a second, less dense tissue or an apparent or potential tissue void, cavity, or vessel. In some embodiments, the energy storage member is configured to exert a force that is less than or equal to the pressure in the first tissue, but greater than the pressure in the second, less dense tissue or an apparent or potential tissue void, cavity, or vessel. In some embodiments, the energy storage member is configured to directly or indirectly exert a force on the floating seal, and the effect of the force is sufficient to overcome the pressure difference between the pressure at the needle distal opening in the supraspinous ligament/interspinous ligament/ligamentum flavum and the pressure at the needle distal opening in an epidural space. Due to the difference in pressure, once the needle distal opening advances through the first tissue and starts to enter the second, less dense tissue, the energy stored in the energy storage member is automatically released to advance the floating seal (e.g., via a piston rod 15 in FIG. 12), thereby discharging a volume of the flowable composition into the second tissue or in a void between the first and second tissues.

Unlike certain existing devices in which a needle is coupled to a floating seal, in some embodiments, the present disclosure utilizes a needle whose proximal end is coupled to an actuation member inside a syringe barrel, where the actuation member is separately provided and is proximal to the floating seal. In some embodiments, the proximal end of a need disclosed herein is not coupled to the floating seal. In some embodiments, prior to use, the needle can be distal to the floating seal or can be through the floating seal, but the proximal end of the needle remains distal to the floating seal and is not fixedly attached to the floating seal.

In certain existing devices, a medicament container (e.g., comprising a liquid) is provided between a proximal seal and a distal seal that each can move within a syringe barrel, for example, as described in US 2020/0069883 which is incorporated herein by reference for all purposes. In those devices, a force on the proximal side of the proximal seal is transmitted through the liquid to the distal seal which is attached to a needle. Given liquids are generally incompressible, when an operator uses too much force or applies a force abruptly on the proximal seal (e.g., through a plug coupled to the proximal seal), the force will be transmitted to the needle. With the liquid providing little compressibility to buffer the impact of the force, the needle may be inserted too deeply or too abruptly, causing damage to the target tissue and/or surrounding tissues (e.g., causing dura puncture). Although the positions of the proximal seal and the distal seal may be observed during injection, once a force that may cause overshooting of the needle is applied, it could already to be too late to stop the movement of the needle due to lack of the ability to buffer the impact of the force.

In contrast, in some embodiments of the present disclosure, the medicament container (e.g., flowable composition lumen) is provided between a floating seal and the distal end of a syringe barrel (where the distal end does not move relative to the syringe barrel). In some embodiments, the distal end of the syringe barrel comprises a distal seal and the flowable composition lumen is provided between the floating seal and the distal seal. In some embodiments, since the needle base is elastically connected to the floating seal (and therefore the flowable composition), the elastic connection can facilitate the operator to apply the right force and buffer the impact of that force. In addition, an operator can hold the needle base still relative to the syringe barrel and observe the movement of the floating seal in order to assess the depth of needle placement. Once fluidic communication is established between the flowable composition and an apparent or potential tissue void, cavity, or vessel, and the pressure in the flowable composition is greater than that in the apparent or potential tissue void, cavity, or vessel, the floating seal can move as the flowable composition enters the tissue, while the needle and the needle base do not have to move. Thus, precise needle placement and steady injection can be achieved and chances of needle overshooting can be effectively reduced or eliminated.

In some embodiments of the present disclosure, the medicament container (e.g., a syringe configured to contain a flowable composition) can be set to have an adjustable volume, e.g., between about 0 and about 0.2 mL, such as between about 0 and about 0.15 mL, particularly between about 0 and about 0.1 mL, including about 0.025 mL, about 0.05 mL, about 0.075 mL, about 0.1 mL, or between any of the aforementioned values. The volume of the flowable composition to be delivered (e.g., via injection) using a device disclosed herein can be selected based on the conditions of a particular subject, and can be adjusted according to changes in the conditions.

In some embodiments, a device disclosed herein is provided and/or packaged as an integrated device comprising components engaging each other. In some embodiments, a device disclosed herein does not require an operator to assemble one or more of components prior to use. In some embodiments, a device disclosed herein comprises a pre-filled medicament container (e.g., flowable composition lumen) comprising a flowable composition, such as a medicament in the form of a liquid, a solution, a suspension, a gel, an oil, an ointment, an emulsion, a cream, a foam, a lotion, and/or a paste.

Flowable compositions include liquid (e.g., solution, suspension, or the like) or semi-solid compositions (e.g., gels) that are easy to manipulate and may be injected, shaped and/or molded at or near the target tissue site as it coagulates. “Flowable” includes formulations with a low viscosity or water-like consistency to those with a high viscosity, such as a viscoelastic or a paste-like material. In some embodiments, a viscoelastic fluid is are a non-Newtonian fluid formed by a viscous component and an elastic one, such as a blend of a solvent and a polymeric material.

In various embodiments, the flowability of the formulation allows it to conform to irregularities, crevices, cracks, and/or voids in the tissue site. For example, in various embodiments, the formulation may be used to fill one or more voids, expand a tissue void (e.g., an apparent tissue void), and/or create a tissue void from a potential tissue void and optionally expand the created void. In some embodiments, upon contact with an aqueous medium (e.g., body fluid, water, etc.), the flowable composition may harden to form a drug depot that controls drug release.

In some embodiments, a therapeutic agent (e.g., a drug) is added to the flowable composition.

In some embodiments, one or more components of a system or device disclosed herein are configured to be assembled with one another. For example, the system or device may comprise one or more syringe barrels.

In some embodiments, the system or device may comprise two or more units, such as a first syringe unit comprising: a first syringe barrel; a needle base in the first syringe barrel; and a needle comprising a needle proximal end engaging the needle base and a needle distal end. In some embodiments, the system or device may comprise a second syringe unit configured to engage a distal end of the first syringe unit, comprising: a second syringe barrel; and a floating seal in the second syringe barrel, and when the first and second syringe units are engaged, the floating seal is configured to elastically engage the needle base. In some embodiments, the system or device may comprise a third syringe unit configured to engage a distal end of the second syringe unit, comprising a third syringe barrel enclosing a flowable composition, and the needle base can be configured to advance the needle to place the needle proximal end and/or the needle distal end in the flowable composition. In any of the embodiments herein, the system or device can comprise one or more syringe units, optionally a fourth syringe unit configured to engage a distal end of the third syringe unit.

In some embodiments, the system or device may comprise a first syringe unit comprising: a first syringe barrel; a needle base and a floating seal in the first syringe barrel elastically engaging each other, the needle base being proximal to the floating seal; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the system or device may further comprise a second syringe unit configured to engage a distal end of the first syringe unit, comprising a second syringe barrel enclosing a flowable composition, and the needle base can be configured to advance the needle to place the needle proximal end and/or the needle distal end in the flowable composition. In any of the embodiments herein, the device can comprise one or more syringe units, optionally a third syringe unit configured to engage a distal end of the second syringe unit.

In some embodiments, the system or device may comprise a first syringe unit comprising: a first syringe barrel; a needle base in the first syringe barrel; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the system or device may further comprise a second syringe unit configured to engage a distal end of the first syringe unit, comprising: a second syringe barrel; a floating seal in the second syringe barrel, and when the first and second syringe units are engaged, the floating seal is configured to elastically engage the needle base; and a flowable composition, and the needle base can be configured to advance the needle to place the needle proximal end and/or the needle distal end in the flowable composition. In any of the embodiments herein, the device can comprise one or more syringe units, optionally a third syringe unit configured to engage a distal end of the second syringe unit.

In some embodiments, the present disclosure provides in an injection system or device comprising: a syringe barrel, wherein the syringe barrel comprises a distal closed end and a proximal open end; an actuation unit (e.g., an elastic movement unit) comprising an actuation member (e.g., pressing element) and a floating seal, wherein the floating seal is positioned inside the syringe barrel and can elastically engage with the actuation member (e.g., pressing element); a hollow puncture needle attached to the actuation member (e.g., pressing element), wherein the hollow puncture needle comprises a needle distal opening and a needle body opening, and wherein the needle body opening is proximal to the floating seal (the needle distal opening can be proximal to the floating seal, e.g., the entire length of the needle is proximal to the floating seal, or alternatively, the needle can be through the floating seal such that the needle distal opening is distal to the floating seal); and a flowable composition lumen (e.g., for a fluid or gel), wherein the flowable composition lumen is formed by the syringe barrel distal closed end, a syringe barrel lumen wall (e.g., a portion of the syringe barrel), and the floating seal.

In some embodiments, the medical puncturing device is configured such that the hollow puncture needle can be moved forward by pressing the actuation member (e.g., pressing element). In some embodiments, the hollow puncture needle sequentially pierces the floating seal and the syringe barrel distal closed end, thus connecting the flowable composition lumen, the needle body opening, and the needle distal opening. In some embodiments, the hollow puncture needle is pre-inserted into the floating seal. For example, the needle distal opening can be in the floating seal and blocked by the floating seal, and the needle can be advanced through the flowable composition lumen to pierce the syringe barrel distal closed end. In some embodiments, the hollow puncture needle is pre-inserted through the floating seal. For example, the needle distal opening can be in the flowable composition lumen, while the needle body opening is proximal to the floating seal or in the floating seal (e.g., the needle body opening can be blocked by the floating seal as shown in FIG. 3E), and then the needle can be advanced to pierce the syringe barrel distal closed end. In some embodiments, the hollow puncture needle is pre-inserted through the floating seal and in or through the syringe barrel distal closed end. For example, the needle distal opening can be in a distal seal at the syringe barrel distal closed end (e.g., the needle distal opening can be blocked by the distal seal) or distal to the distal seal and/or the syringe barrel distal closed end, while the needle body opening is proximal to the floating seal (e.g., as shown in FIGS. 3D, 6b1), in the floating seal (e.g., the needle body opening can be blocked by the floating seal as shown in FIGS. 3D, 6b2), or in the flowable composition lumen (e.g., as shown in FIGS. 3D, 6b3), and then the needle can be advanced through the syringe barrel distal closed end and exposing the needle distal opening for puncturing a tissue.

Optionally, the medical puncturing device comprises a state wherein the flowable composition lumen, the needle body opening, and the needle distal opening are in fluidic communication. For example, in a fluidic communication state, the needle body opening can be proximal to the floating seal, while the needle distal opening is distal to the floating seal and in the flowable composition lumen. In the fluidic communication state, the needle and/or the floating seal can be moved. For example, the floating seal can be moved under the elastic resilience between the floating seal and the actuation member (e.g., pressing element) such as that the floating seal seals or blocks the needle body opening, thereby preventing or terminating discharge of the flowable composition (such as a gel) from the needle body opening and/or from the needle distal opening.

Optionally, in the fluidic communication state, the floating seal can seal the needle body opening when it moves forward and contacts the syringe barrel distal closed end, thereby preventing or terminating discharge of the flowable composition (such as a gel) from the needle body opening and/or from the needle distal opening.

Optionally, a stopper such as an axial stopper can be provided inside the syringe lumen, distal to the floating seal. In some embodiments, the stopper can be used to limit the forward movement of the floating seal. In some embodiments, the medical puncturing device comprises a fluidic communication state, wherein the flowable composition lumen is connected to the needle body opening and the needle distal opening. When the medical puncturing device is in the fluidic communication state, the needle body opening can be at the distal end of the stopper (e.g., as shown in FIG. 2D), and the floating seal can move forward due to the elastic engagement with the actuation member (e.g., pressing element).

Optionally, the medical puncturing device comprises a manual control element, which is attached to the floating seal and is extended outside of the syringe barrel.

Optionally, the medical puncturing device comprises a pre-puncture state after the hollow puncture needle pierces the syringe barrel distal closed end, a surface tissue puncture state, and a fluidic communication state after the puncture. In the pre-puncture state, the surface tissue puncture state, and the fluidic communication state, the length range of the hollow puncture needle extended outside of the syringe barrel distal closed end can correspond to a pre-puncture length range, a surface tissue puncture length range, and a fluidic communication length range, respectively, wherein: when the length of the of the hollow puncture needle extended outside of the syringe barrel distal closed end is within the pre-puncture length range, the needle body opening remains above the flowable composition lumen (e.g., the needle body opening can be proximal to and within the floating seal); and/or when the length of the of the hollow puncture needle extended outside of the syringe barrel distal closed end is within the surface tissue puncture length range, at least part of the needle body opening is connected to the flowable composition lumen; and/or, when the length of the of the hollow puncture needle extended outside of the syringe barrel distal closed end is within the fluidic communication length range, the needle body opening is positioned within the flowable composition lumen.

Optionally, an axially extended circular contacting element is formed at the syringe barrel distal closed end, wherein the difference between the upper and lower limits of the pre-puncture length range equals to the axial length of the circular contacting element.

Optionally, the elastic movement unit comprises a elastic sheath covering the outside of the hollow puncture needle. When the needle body opening is proximal to the floating seal, the elastic sheath can seal the needle body opening. In some embodiments, when the flowable composition is a gel, it may not be necessary to seal the needle body opening when it is proximal to the floating seal.

Optionally, the medical puncturing device comprises a catheter guiding structure which is used to thread the catheter into a cavity such as the epidural space (e.g., a needle body passageway connected to the needle distal opening and/or the needle body opening) of the hollow puncture needle.

Optionally, the catheter guiding structure comprises an angled guiding groove which is formed on the floating seal and extends towards the hollow puncture needle in an angle.

Optionally, the angled guiding groove is set to be through the floating seal in the front and back direction. In some embodiments, the catheter guiding structure further comprises a one-way valve which is embedded in the angled guiding groove and can be opened and closed, and/or a guiding groove plug inserted in the angled guiding groove.

Optionally, the angled guiding groove is set to be on the upper surface of the floating seal and is a non-through groove.

Optionally, the needle body opening is formed as an angled opening which opens obliquely backwards.

Optionally, the catheter guiding structure comprises an angled guiding needle hole formed on the body wall of the hollow puncture needle and opens obliquely backwards. In some embodiments, the medical puncturing device comprises a fluidic communication state wherein the flowable composition lumen is in connection with the needle body opening and the needle distal opening. In the fluidic communication state, the angled guiding needle hole is positioned proximal to the floating seal.

Optionally, the catheter guiding structure further comprises a one-way valve which is embedded in the angled guiding needle hole and can be opened and closed, or a guiding groove plug inserted in the angled guiding needle hole.

Optionally, the catheter guiding structure comprises a puncturable central guiding groove that is formed on the center of the proximal surface of the actuation member (e.g., pressing element). In some embodiments, a needle proximal opening is formed on the hollow puncture needle and the needle proximal opening is set to axially align with the central guiding groove.

Optionally, the medical puncturing device comprises a puncture control module and a fluid storage module that are independently manufactured and formed, wherein: the puncture control module comprises a first syringe unit and the elastic movement unit and the hollow puncture needle provided inside the first syringe unit; the fluid storage module comprises a second syringe unit, the flowable composition lumen formed inside the barrel of the second syringe unit, and a module packaging component which is removably packaged to the proximal end of the second syringe unit; and a removable connection structure is formed between the first syringe unit and the second syringe unit.

In a second aspect, the present disclosure provides a medical apparatus assembly. In some embodiments, the medical apparatus assembly comprises a catheter and the medical puncturing device comprising a catheter guiding structure.

Optionally, the medical apparatus assembly further comprises a hollow auxiliary guiding needle which is matched to use with the catheter guiding structure. In some embodiments, when the auxiliary guiding needle is connected to the catheter guiding structure, the catheter can sequentially go through the needle body passageway of the auxiliary guiding needle and the catheter guiding structure and be threaded into the needle body passageway of the hollow puncture needle.

In some embodiments, when using the medical puncturing device of the present disclosure, a user can first apply pressure to the actuation member (e.g., pressing element) to drive the hollow puncture needle sequentially through the floating seal and the syringe barrel distal closed end. When the needle distal opening of the hollow puncture needle reaches apparent or potential tissue gaps, cavity systems, and vessels (e.g., the epidural space), the needle body opening has already been positioned in the flowable composition lumen, and the floating seal has already formed an elastic engagement with the actuation member (e.g., pressing element). In some embodiments, the fluid pressure in the flowable composition lumen can be made higher than the pressure inside the an apparent or potential tissue void, cavity, or vessel.

At this time, the fluid inside the flowable composition lumen can flow into the an apparent or potential tissue void, cavity, or vessel through the needle body opening and the needle distal opening. During the injection process, just by maintaining the position of the actuation member (e.g., pressing element), under the action of the elastic engagement between the floating seal and the actuation member (e.g., pressing element), the fluid inside the flowable composition lumen can flow into the needle body opening (and then through the needle body passageway and out of the needle distal opening), thereby achieving injection, penetration, and/or expansion of the an apparent or potential tissue void, cavity, or vessel. Additionally, the medical apparatus assembly as describe in the present disclosure can achieve implantation of catheter and other medical device through the medical puncturing device, e.g., through a catheter guiding structure and a cavity of the needle described herein.

In some embodiments, before the hollow puncture needle pierces into an apparent or potential tissue void, cavity, or vessel, the external pressure on the needle distal opening is higher than the fluid pressure in the flowable composition lumen, thus fluid cannot flow out of the needle distal opening. Thus, by observing whether the floating seal moves forward due to the elastic engagement with the actuation member (e.g., pressing element), it is possible to determine whether the hollow puncture needle has already pierced into an apparent or potential tissue void, cavity, or vessel, thereby reminding the operator of the current punctuation depth to ensure accurate puncture. Since the injection is controlled by fluid pressure changes in the flowable composition lumen, the injection process does not require an operator to manually apply thrust or force during the injection process, thus fluctuations in the flow speed can be prevented and stable injection can be achieved.

In some embodiments, provided herein is a method of providing epidural injection and/or placing an implant (e.g., catheter) into the epidural space, comprising using an injection system, wherein the injection system comprises:
a syringe barrel extending from a proximal end to a distal end;
a first hollow needle extending from a proximal end to a distal end comprising an end opening, wherein the needle distal end is connected to the distal end of the syringe barrel;
a floating seal, wherein the floating seal is positioned inside the syringe barrel, forms a lumen between the floating seal and the distal end of the syringe barrel, and comprises a hollow channel aligned with the first hollow needle;
a push shaft with a hollow channel extending from a proximal end to a distal end, wherein the distal end of the push shaft is proximal to and in contact with the floating seal, and the hollow channel of the push shaft is aligned with the hollow channel of the floating seal and the first hollow needle to form a central hollow channel extending from the proximal end of the push shaft to the distal opening of the first hollow needle;
a proximal seal at the proximal end of the central hollow channel; and
an actuation unit comprising an actuation member and an energy storage member, wherein the actuation member can elastically engage with the push shaft via the energy storage member.

In some embodiments of the foregoing, the injection system further comprises a second syringe unit (e.g., 50 in FIG. 15) comprising a needle extending from a proximal end to a distal end, wherein the needle distal end of the second syringe can pierce the proximal seal, and the needle of the second syringe unit can be placed into the central hollow channel. In some embodiments, the injection system further comprises a piercing unit (e.g., 6′ in FIG. 14B and FIG. 15), wherein the piercing unit can pierce the proximal seal (e.g., 8a in FIG. 14B and FIG. 15), and allow the needle of the second syringe unit can be placed into the central hollow channel. In some embodiments, when the needle of the second syringe is placed into the central hollow channel, the distal end of the needle of the second syringe unit can be distal to the distal opening of the first hollow needle. In some embodiments, the second syringe unit comprises a fluid lumen containing a flowable material, wherein the flowable material can be injected in an epidural space via the needle of the second syringe, when the distal end of the first hollow needle is at a target injection site. In some embodiments, the needle of the second syringe can be withdrawn from the central hollow channel when the injection of a flowable material is finished. In some embodiments, the injection system further comprises a needle guiding structure with a hollow needle guiding channel extending from a proximal end to a distal end in the guiding structure 43, wherein the distal end of the needle guiding channel is connected to and aligned with the proximal end of the central hollow channel. In some embodiments, which may be combined with any of the foregoing embodiments, the needle of the second syringe unit can be inserted into the central hollow channel via the needle guiding channel, and the needle guiding channel can stabilized the needle of the second syringe unit.

In some embodiments, an implant (e.g., a catheter) can be placed into an epidural space via the central hollow channel. In some embodiments, the injection system further comprises a piercing unit (e.g., 6′ in FIG. 14B and FIG. 15), wherein the piercing unit can pierce the proximal seal, and allow the catheter to be placed into the central hollow channel. In some embodiments, the injection system further comprises a catheter guiding structure with a hollow catheter guiding channel extending from a proximal end to a distal end (e.g., 12b in FIG. 14B and FIG. 15), wherein the distal end of the catheter guiding channel is connected to and aligned with the proximal end of the central hollow channel. In some embodiments, when the piercing unit pierces the proximal seal, a catheter (e.g., 11 in FIG. 14B and FIG. 15) can be placed into the central hollow channel via the catheter guiding channel.

In some embodiments, the guiding structure 43 can comprise a side port 44, such that when the guiding structure 43 is advanced distally, the side port aligns with a catheter guiding structure with a hollow channel (e.g., 12b in FIG. 14B and FIG. 15), and a catheter can be inserted through the side port into the hollow needle guiding channel inside the guiding structure 43 and further inserted into the central hollow channel inside push shaft 2, into the hollow puncture needle 6, and eventually into an epidural space.

In some embodiments, the injection system further comprises a needle stabilizing structure connected to the catheter (e.g., 12a in FIG. 14B and FIG. 15), preferably to the proximal side of the catheter, so that the catheter can be stabilized during insertion. In some embodiments, during the insertion of a catheter into the central hollow channel, the catheter is moved distally by hand. In some embodiments, the injection system further comprises a catheter insertion unit (e.g., 42 in FIG. 14B and FIG. 15), and during the insertion of a catheter into the central hollow channel, the catheter is moved distally by the catheter insertion unit.

In some embodiments, the injection further comprises both a needle guiding structure with a hollow channel (e.g., 43 in FIG. 14B and FIG. 15), which forms a hollow channel with the central hollow channel for needle of a second syringe, and a catheter guiding structure with a hollow channel (e.g., 12b in FIG. 14B and FIG. 15), which forms a hollow channel with the central guiding channel for a catheter. In some embodiments, the side wall of the needle guiding structure comprises an opening, and an angled hollow catheter guiding structure (e.g., 12b in FIG. 14B and FIG. 15) can be connected to the opening, further to the needle guiding structure, and further to the central hollow channel, so that a hollow channel is formed for catheter insertion. In some embodiments, the needle of the second syringe can pierce the proximal seal, and simultaneously opens the needle guiding channel and the catheter guiding channel. In some embodiments, the piercing unit can pierce the proximal seal, and simultaneously opens the needle guiding channel and the catheter guiding channel.

In some embodiments, provided herein is an integrated device. The device comprises a) a syringe barrel (e.g., 1 in FIG. 17) extending from a proximal end to a distal end, wherein the distal end of the syringe barrel comprises a needle base (e.g., 4 in FIG. 17) configured to be coupled to a needle (e.g., 5 in FIG. 17) comprising a needle lumen (e.g., 6 in FIG. 17), and wherein the needle base comprises a passageway (e.g., 7 in FIG. 17) configured to fluidically communicate with the needle lumen; a gasket seal (e.g., 3 in FIG. 17) that forms a fluid-tight seal with an inner wall of the syringe barrel, wherein a flowable composition lumen (e.g., 8 in FIG. 17) is formed between the gasket seal and the distal end of the syringe barrel, wherein the gasket seal comprises a through hole (e.g., 9 in FIG. 17), and wherein the through hole is configured to align with the passageway in the needle base; c) a push shaft (e.g., 2 in FIG. 17) extending from a proximal end to a distal end, wherein the distal end of the push shaft engages the gasket seal, and the push shaft comprises: a central channel (e.g., 10 in FIG. 17) comprising a distal end that aligns with the through hole of the gasket seal; and a valve (e.g., 11 in FIG. 17) that aligns with a proximal end of the central channel; d) an elastic element (e.g., 12 in FIG. 17) configured to actuate the push shaft such that the gasket seal is moved distally; and e) a catheter configured to be inserted through the valve and into the central channel. In some embodiments, the gasket seal is configured to move distally (e.g., along the axis of the syringe barrel) to connect the central channel and the passageway in the needle base. For instance, the central channel in the push shaft can be a tubular structure that is configured to pierce the gasket seal or pass through the through hole (of the gasket seal) which can be along an axis of the syringe barrel. In some embodiments, the gasket seal is configured to move distally (e.g., along the axis of the syringe barrel) to abut the central channel and the passageway in the needle base.

Having an integrated device offers many advantages over assembling a device from separate components. (1) The integrated device ensures that all parts are optimized to work seamlessly together, enhancing overall reliability, performance, and user experience. This holistic approach allows for more rigorous quality control, as the device is conceived and manufactured as a single unit, reducing the chances of incompatibility or failure between individual parts. It also streamlines the regulatory approval process, since the integrated device can be evaluated as a whole rather than individual parts. (2) The integrated device is also user-friendly and intuitive, making it easier for healthcare providers to be trained on and operate the device. An integrated device not only eliminates the redundancies and inefficiencies that can occur when combining components from different sources, but it also creates a less stressful environment in the fast-paced and high-stakes setting of a hospital. The result is a medical device that is more coherent, efficient, and effective, contributing to quicker and safer healthcare delivery.

In some embodiments, the integrated device comprises a needle (e.g., 5 in FIG. 17). In some embodiments, the needle is an epidural needle. In some embodiments, the epidural needles can be used to administer epidural an anesthesia or an analgesia during a labor, a childbirth, a surgery, a diagnostic procedure, a pain control, or other medical conditions where a dose of medication is required in the epidural space. In some embodiments, the needle is a Tuohy epidural needle, a Hustead epidural needle, a Crawford epidural needle, or a Weiss epidural needle. These specialized epidural needles are designed for epidural anesthesia or analgesia procedures with specific features intended to improve the ease, safety, and effectiveness of the procedure. In some embodiments, the needle is between about 17 G and about 22 G (iso-9626) in gauge size. In some embodiments, the needle is 17-18 G (iso-9626) in gauge size. In some embodiments, the needle is 19-20 G (iso-9626) in gauge size. “G” stands for gauge, which is a measure of the thickness of the needle. The gauge size of an epidural needle can generally range between 17 G and 22 G as per ISO-9626 standards. They are chosen based on a balance of several factors, e.g. ease of insertion, minimizing complications, flow rate, visibility, patient comfort, durability etc. In some embodiments, the needle is between about 2.5 and about 6 inches in length. In some embodiments, the needle is between about 3 and about 3.5 inches in length. The length of an epidural needle is designed to reach the epidural space effectively while minimizing the risk of complications. Factors that influence this choice of needle length can be anatomical considerations, safety margin, ese of maneuverability, or clinical experience etc. In some embodiments, the needle comprises a straight distal tip. In some embodiments, the needle comprises a curved distal tip. In some embodiments, the needle comprises a distal tip comprising a blunt bevel. The design of the distal tip of an epidural needle can vary based on specific medical needs, procedure types, and physician preferences. Different tips offer different advantages and limitations: In some embodiments, the needle comprises a side port. In some embodiments, the side port of the needle is configured to allow injection of a flowable composition through the needle lumen (e.g., 6 in FIG. 17). In some embodiments, the side port of the needle is configured to allow injection of an anesthetic agent through the needle lumen. Based on the disclose above, a person having ordinary skills would know how to appropriately choose a suitable needle in operations with the integrated device (e.g., FIGS. 16 and 17) in order to maximize effectiveness while minimizing complications and risks.

In some embodiments, the integrated device comprises a gasket seal (e.g., 3 in FIG. 17). In some embodiments, the gasket seal is configured to allow distal advancement of the catheter through the through hole (e.g., 9 in FIG. 17). In some embodiments, the gasket seal is configured to allow proximal retraction of the catheter through the through hole. In some embodiments, the gasket seal is configured to prevent proximal retraction of the catheter through the through hole. In some embodiments, the gasket seal is configured to lock the catheter in place through the through hole so the catheter is prevented from either a proximal retraction or a distal advancement. Depending on the specific requirements of the medical procedure being performed, each of these embodiments can be further configured to move an additional distance for fine adjustments after the catheter is locked in place, e.g. 0-about 10 centimeters distally or proximally. In some embodiments, the through hole (e.g., 9 in FIG. 17) is configured to close in the absence of the catheter or the central channel (e.g., 10 in FIG. 17) inserted in the gasket seal. In some embodiments, the through hole is configured to remain open in the absence of the catheter or the central channel inserted in the gasket seal. In some embodiments, the through hole is configured to be sealed in the absence of the catheter or the central channel inserted in the gasket seal. In some embodiments, the through hole is configured to allow the catheter or the central channel to pass through the gasket seal. Each of these configurations caters to specific clinical scenarios, usability concerns, and safety protocols, in maintaining a controlled environment within a body cavity or surgical site. For example, a sealed through hole of the gasket seal can prevent an unintended fluid leakage or a backflow in cases where some patients may have an unusual high pressure in their epidural space.

In some embodiments, the integrated device comprises a central channel (e.g., 10 in FIG. 17). In some embodiments, distinct configurations for how the central channel interacts with the through hole (e.g., 9 in FIG. 17) of the gasket seal (e.g., 3 in FIG. 17) can cater to specific clinical needs and procedural requirements. In some embodiments, a distal end of the central channel (e.g., 10 in FIG. 17) passes through or abuts the through hole of the gasket seal. In some embodiments, the distal end of the central channel extends distally beyond the gasket seal. In some embodiments, the distal end of the central channel extends distally beyond the gasket seal and can be placed inside the passageway (e.g., 7 in FIG. 17) of the needle base (e.g., 4 in FIG. 17), thereby reducing the likelihood of the catheter being stuck at the interface of the needle base and the central channel, e.g. in particularly when a thin and soft catheter is used with the integrated device. In some embodiments, the configuration of the distal end of the central channel to pass entirely through the through-hole can enhance ease of insertion of the catheter and improve on the reproducibility. In some embodiments, the distal end of the central channel is flush with the distal end of the through hole. In some embodiments, the distal end of the central channel is flush with the proximal end of the through hole. These alternative configurations of having the distal end of the central channel (e.g., 10 in FIG. 17) flush with either the proximal end or the distal end of the through hole (e.g., 9 in FIG. 17) can greatly reduce the manufacturing cost of the integrated device by not having the central channel extension in the integrated device.

In some embodiments, the integrated device comprises a valve (e.g., 11 in FIG. 17) at the proximal end of the central channel (e.g., 10 in FIG. 17). In some embodiments, the push shaft (e.g., 2 in FIG. 17) further comprises a central chamber (e.g., 20 in FIG. 17) in fluidic communication with the central channel (e.g., 10 in FIG. 17). The central chamber is optional and in some embodiments, the push shaft does not comprise any chamber or channel except the central channel which is configured to accommodate at least a portion of a catheter. In some embodiments, the central chamber is at least partially between the valve and the proximal end of the central channel. In some embodiments, the central chamber acts as a buffering space and allows the valve to open upon insertion of the catheter. In some embodiments, the central chamber provides a buffering space for materials forming the valve to open distally into the central chamber. In some embodiments, the valve is configured to allow one-way passage of the catheter. In some embodiments, the valve is configured to allow two-way passage of the catheter. In some embodiments, the valve is configured to lock the catheter in place. In some embodiments, the valve is configured to allow the catheter to make further fine movements, e.g. 0-about 10 centimeters distally or proximally, after the catheter is locked in place. In some embodiments, the push shaft comprises a catheter guiding channel (e.g., 13 in FIG. 17). In some embodiments, the valve is at the distal end of the catheter guiding channel. In some embodiments, the inner diameter of the catheter guiding channel is 20% larger than the outer diameter of the catheter, thereby reducing the resistance during the movement of the catheter in the catheter guiding channel. In some embodiments, the valve aligns with a distal end of the catheter guiding channel. In some embodiments, the inner wall of the open valve is flush with the inner wall of the catheter guiding channel, thereby preventing the catheter from being stuck at the interface when passing through the valve. In some embodiments, the push shaft comprises a side port (e.g., 19 in FIG. 17) configured to allow passage of the catheter through the side port into the catheter guiding channel. In some embodiments, the passageway of the side port forms a blunt angle with the catheter guiding channel, thereby allowing the catheter to be inserted with ease. In some embodiments, the push shaft comprises a locking mechanism configured to maintain a position of the gasket seal in the syringe barrel and a compressed or expanded state of the elastic element (e.g., 12 in FIG. 17). In some embodiments, the push shaft can be proximally withdrawn and compress the elastic element in the process. In some embodiments, the locking mechanism allows the push shaft to maintain at a proximal position, wherein the elastic element maintains a compressed state. In some embodiments, the push shaft can be proximally withdrawn and expand the elastic element in the process. In some embodiments, the locking mechanism allows the push shaft to maintain at a proximal position, wherein the elastic element maintains an expanded state. In some embodiments, the locking mechanism is activated by turning the push shaft clockwise or counterclockwise. In some embodiments, the turning angle of activating the locking mechanism is 90 degree. In some embodiments, the locking mechanism is deactivated by turning the push shaft clockwise or counterclockwise. In some embodiments, the turning angle of deactivating the locking mechanism is 90 degree. In some embodiments, a proximal position of the push shaft is maintained by activating the locking mechanism. In some embodiments, a proximal position of the push shaft is maintained not by activating the locking mechanism, instead by a high pressure present in the flowable composition lumen (e.g., 12 in FIG. 17). In some embodiments, the high pressure present in the flowable composition lumen is caused by the distal end of the needle in the anatomical tissue layers, e.g. the skin, subcutaneous, supraspinous ligament, interspinous ligament, or ligamentum flavum. In some embodiments, the high pressure present in the flowable composition lumen drops upon the distal end of the needle enters the epidural space. In some embodiments, the pressure drop of the flowable composition lumen allows the push shaft (e.g., 2 in FIG. 17) or the gasket seal (e.g., 3 in FIG. 17) to move distally toward the needle base (e.g., 4 in FIG. 17).

In some embodiments, the integrated device comprises an elastic element (e.g., 12 in FIG. 17). The elastic element provides a means to convert stored potential energy (from its compressed or expanded state) into kinetic energy, resulting in the movement or actuation of other components in the device, e.g. the push shaft (e.g., 2 in FIG. 17) or the gasket seal (e.g., 3 in FIG. 17). In some embodiments, the elastic element comprises a spring, a rubber band, a bungee cord, a memory foam, an air bag, or a combination thereof. In some embodiments, a distal end of the elastic element engages a portion of the push shaft or the gasket seal, and a proximal end of the elastic element engages a portion of the syringe barrel. In some embodiments, the elastic element is configured to be compressed, and wherein the decompression of the compressed elastic element exerts a force to actuate the push shaft or the gasket seal such that the gasket seal is moved distally along the axis of the syringe barrel. In some embodiments, a distal end of the elastic element engages a portion of the syringe barrel, and a proximal end of the elastic element engages a portion of the push shaft or the gasket seal. In some embodiments, the elastic element is configured to be expanded, and wherein the collapse of the expanded elastic element exerts a force to actuate the push shaft or the gasket seal such that the gasket seal is moved distally along the axis of the syringe barrel (e.g., 1 in FIG. 17). In some embodiments, the elastic element serves as a mechanism for generating force or movement within the integrated device, specifically for actuating the push shaft or the gasket seal in a way that moves the gasket seal along the axis of the syringe barrel.

In some embodiments, a catheter (e.g., 16 in FIG. 17) is used with the integrated device. In some embodiments, the catheter is between about 19 G and about 20 G (iso-9626) in gauge size. In some embodiments, the outer surface of the catheter is modified with a feature to indicate a position of the catheter in the relative to the integrated device. In some embodiments, the feature comprises an expansion, a depression, a hump, a bent, a knot, a groove, a marking, or a joint. In some embodiments, the integrated device further comprises a housing (e.g., 40 in FIG. 17) accommodating at least a portion of the syringe barrel, at least a portion of the push shaft, and at least a portion of the catheter. The housing provides a protective enclosure for the integrated device's internal components and ensures ease of use and comfort for both the medical professional and the patient. In some embodiments, the integrated device further comprises a catheter storage mechanism (e.g., 43 in FIG. 17) and a catheter actuation mechanism (e.g., 42 in FIG. 17). In some embodiments, at least a portion of the catheter is inserted through the valve (e.g., 11 in FIG. 17). In some embodiments, at least a portion of the catheter is inserted into the central channel (e.g., 10 in FIG. 17). In some embodiments, at least a portion of the catheter is passed through the through hole (e.g., 9 in FIG. 17) of the gasket seal. In some embodiments, at least a portion of the catheter is passed through the passageway (e.g., 4 in FIG. 17) of the needle base. In some embodiments, the distal advancement of the catheter (e.g., 16 in FIG. 17) is controlled by a catheter insertion unit (e.g., 42 in FIG. 17). In some embodiments, the catheter insertion unit is a pair of gears wherein the catheter is located in between the two gears. In some embodiments, the distal advancement of the catheter is achieved by dialing the gears. In some embodiments, at least a portion of the catheter is inserted into the needle lumen (e.g., 6 in FIG. 17). In some embodiments, the catheter comprises a distal end configured to form a coil, thereby reducing a risk of the tip of the catheter moving away from the insertion site. The integration and interaction of the catheter with the other components of the integrated device are tailored to ensure smooth, efficient, and safe operation, catering to various medical applications and needs.

In some embodiments, provided herein is an integrated device. In some embodiments, the integrated device comprises a syringe barrel (e.g., 1 in FIG. 17), a gasket seal (e.g., 3 in FIG. 17), a push shaft (e.g., 2 in FIG. 17), a spring (e.g., 12 in FIG. 17), a catheter (e.g., 16 in FIG. 17), and a housing (e.g., 40 in FIG. 17). In some embodiments, the syringe barrel extends from a proximal end to a distal end, wherein the distal end of the syringe barrel comprises a needle base coupled to a needle comprising a needle lumen. In some embodiments, the needle base comprises a passageway fluidically communicating with the needle lumen. In some embodiments, the gasket seal forms a fluid-tight seal with an inner wall of the syringe barrel, wherein the gasket seal abuts the distal end of the syringe barrel. In some embodiments, the gasket seal comprises a through hole, and wherein the through hole aligns with the passageway in the needle base. In some embodiments, the through hole is along an axis of the syringe barrel. In some embodiments, the push shaft extends from a proximal end to a distal end, wherein the distal end of the push shaft engages the gasket seal. In some embodiments, the push shaft comprises a central channel. In some embodiments, the central channel comprises a distal end that aligns with the through hole of the gasket seal and a valve that aligns with a proximal end of the central channel. In some embodiments, a distal end of the spring engages a portion of the push shaft, and a proximal end of the spring engages a structure (e.g., a baffle) in or of the syringe barrel, wherein the spring is configured to be compressed, and decompression of the compressed spring actuates the push shaft or the gasket seal such that the gasket seal is moved distally along the axis of the syringe barrel. In some embodiments, the catheter is configured to be inserted through the valve and into the central channel. In some embodiments, the housing accommodates at least a portion of the syringe barrel, at least a portion of the push shaft, and at least a portion of the catheter.

In some embodiments, provided herein is an integrated device. In some embodiments, the integrated device comprises a syringe barrel (e.g., 1 in FIG. 17), a gasket seal (e.g., 3 in FIG. 17), a push shaft (e.g., 2 in FIG. 17), a spring (e.g., 12 in FIG. 17), a catheter (e.g., 16 in FIG. 17), and a housing (e.g., 40 in FIG. 17). In some embodiments, the syringe barrel extending from a proximal end to a distal end, wherein the distal end of the syringe barrel comprises a needle base coupled to a needle comprising a needle lumen. In some embodiments, the needle base comprises a passageway fluidically communicating with the needle lumen. In some embodiments, the gasket seal forms a fluid-tight seal with an inner wall of the syringe barrel, wherein a flowable composition lumen is formed between the gasket seal and the distal end of the syringe barrel. In some embodiments, the gasket seal comprises a through hole, and wherein the through hole aligns with the passageway in the needle base. In some embodiments, the push shaft extends from a proximal end to a distal end, wherein the distal end of the push shaft engages the gasket seal. In some embodiments, the push shaft comprises a central channel comprising a distal end that aligns with the through hole of the gasket seal and a valve that aligns with a proximal end of the central channel. In some embodiments, the distal end of the spring engages a portion of the push shaft, and a proximal end of the spring engages a structure (e.g., a baffle) in or of the syringe barrel, wherein the spring is compressed, and decompression of the compressed spring actuates the push shaft or the gasket seal such that the gasket seal is moved distally along the axis of the syringe barrel. In some embodiments, the catheter is configured to be inserted through the valve and into the central channel. In some embodiments, the housing accommodates at least a portion of the syringe barrel, at least a portion of the push shaft, and at least a portion of the catheter. In some embodiments, the flowable composition lumen contains a gas, optionally wherein the gas is air. In some embodiments, the flowable composition lumen contains no liquid.

In some embodiments, provided herein is an integrated device. In some embodiments, the integrated device comprises a syringe barrel (e.g., 1 in FIG. 17), a gasket seal (e.g., 3 in FIG. 17), a push shaft (e.g., 2 in FIG. 17), a spring (e.g., 12 in FIG. 17), a catheter (e.g., 16 in FIG. 17), and a housing (e.g., 40 in FIG. 17). In some embodiments, the syringe barrel extends from a proximal end to a distal end, wherein the distal end of the syringe barrel comprises a needle base coupled to a needle comprising a needle lumen. In some embodiments, the needle base comprises a passageway fluidically communicating with the needle lumen. In some embodiments, the gasket seal forms a fluid-tight seal with an inner wall of the syringe barrel. In some embodiments, the gasket seal comprises a through hole, and wherein the through hole aligns with the passageway in the needle base. In some embodiments, the through hole is along an axis of the syringe barrel. In some embodiments, the push shaft extends from a proximal end to a distal end, wherein the distal end of the push shaft engages the gasket seal. In some embodiments, the push shaft comprises a central channel comprising a distal end that aligns with the through hole of the gasket seal and a valve that aligns with a proximal end of the central channel. In some embodiments, a distal end of the spring engages a portion of the push shaft, and a proximal end of the spring engages a structure (e.g., a baffle) in or of the syringe barrel, wherein the spring exerts a force on the push shaft or the gasket seal such that the gasket seal abuts the distal end of the syringe barrel, and wherein the central channel and the passageway in the needle base are configured to be connected. In some embodiments, the catheter is configured to be inserted through the valve and into the central channel. In some embodiments, the housing accommodates at least a portion of the syringe barrel, at least a portion of the push shaft, and at least a portion of the catheter.

In some embodiments, provided herein is an integrated device. In some embodiments, the integrated device comprises a syringe barrel (e.g., 1 in FIG. 17), a gasket seal (e.g., 3 in FIG. 17), a push shaft (e.g., 2 in FIG. 17), a spring (e.g., 12 in FIG. 17), a catheter (e.g., 16 in FIG. 17), and a housing (e.g., 40 in FIG. 17). In some embodiments, the syringe barrel extends from a proximal end to a distal end, wherein the distal end of the syringe barrel comprises a needle base coupled to a needle comprising a needle lumen. In some embodiments, the needle base comprises a passageway fluidically communicating with the needle lumen. In some embodiments, the gasket seal forms a fluid-tight seal with an inner wall of the syringe barrel, wherein the gasket seal comprises a through hole, and wherein the through hole aligns with the passageway in the needle base. In some embodiments, the through hole is along an axis of the syringe barrel. In some embodiments, the push shaft extends from a proximal end to a distal end, wherein the distal end of the push shaft engages the gasket seal. In some embodiments, the push shaft comprises a central channel comprising a distal end that aligns with the through hole of the gasket seal; a valve that aligns with a proximal end of the central channel; and a catheter guiding channel. In some embodiments, the valve aligns with a distal end of the catheter guiding channel. In some embodiments, a distal end of the spring engages a portion of the push shaft, and a proximal end of the spring engages a structure (e.g., a baffle) in or of the syringe barrel, wherein the spring exerts a force on the push shaft or gasket seal such that the gasket seal abuts the distal end of the syringe barrel, and wherein the central channel and the passageway in the needle base are configured to be connected. In some embodiments, a portion of the catheter is in the catheter guiding channel. In some embodiments, the housing accommodates at least a portion of the syringe barrel, at least a portion of the push shaft, and at least a portion of the catheter. In some embodiments, the distal end of catheter is in the catheter guiding channel of the push shaft. In some embodiments, wherein the distal end of catheter is inserted through the valve of the push shaft. In some embodiments, the distal end of catheter is in the central channel of the push shaft or the through hole of the gasket seal. In some embodiments, the distal end of catheter is in the passageway of the needle base. In some embodiments, the distal end of catheter is in the needle lumen of the needle.

### III. Methods for Medical Penetration

In some embodiments, disclosed herein is a method of epidural injection, comprising using the injection system described in any of the embodiments herein to inject anesthetic into the epidural space. In some embodiments, disclosed herein is a method of using the injection system described in any of the embodiments herein to place a catheter into the epidural space and inject anesthetic into the epidural space through the catheter.

In some embodiments, described herein are methods for medical puncture, for example, in an epidural space or other organs or tissues. In some embodiments, the method disclosed herein can also be used to administer a drug into an intraspinal space in an intraspinal anesthesia. In some embodiments, the epidural anesthesia puncture device disclosed herein can also be used to administer a drug into an intrathecal space in an intrathecal anesthesia.

In some embodiments the present disclosure provides a medical puncturing or penetration device which comprises syringe barrel 1, an actuation unit (e.g., an elastic movement unit for pushing a needle), hollow puncture needle 6, and flowable composition lumen 7.

In some embodiments, syringe barrel 1 comprises a distal closed end and a proximal open end. In some embodiments, syringe barrel 1 can be designed to have two open ends in an axial direction, and sealing of the distal end can be achieved by installing distal seal 8 at the distal opening of syringe barrel 1. In some embodiments, distal seal 8 can be made of a material that can be punctured by hollow puncture needle 6, such as rubber or the like.

In some embodiments, the actuation unit (e.g., elastic movement unit) comprises actuation member (e.g., pressing element) 2 and floating seal 3, where the floating seal 3 sealingly engages an inside wall of the syringe barrel and is configured to move in an axial direction, e.g., toward the distal end or the proximal end of the syringe barrel. In some embodiments, actuation member (e.g., pressing element) 2 or a portion thereof is located outside the proximal opening of the syringe barrel, so that an operator can press on the actuation member (e.g., pressing element) or portion thereof manually. In some embodiments, floating seal 3 elastically engages actuation member 2, and when pressure is applied on actuation member 2, floating seal 3 can move forward or backward relative to the actuation member (e.g., pressing element). In some embodiments, floating seal 3 is configured to move toward the distal end of the syringe barrel. In some embodiments, floating seal 3 is configured to move toward the proximal end of the syringe barrel. In some embodiments, the position of the actuation member (e.g., pressing element) relative to the syringe barrel is kept still, floating seal 3 is configured to move forward (e.g., in a distal direction) under elastic resilience due to the elastic engagement with the actuation member (e.g., pressing element).

In some embodiments, hollow puncture needle 6 is fixedly connected to actuation member 2. When no pressure is applied to actuation member 2, hollow puncture needle 6 remains proximal to floating seal 3 and the two do not come into contact. In some embodiments, hollow puncture needle 6 itself comprises needle distal opening 6a and needle body opening 6b. In some embodiments, needle distal opening 6a and needle body opening 6b are connected through a needle cavity or needle body passageway of hollow puncture needle 6.

In some embodiments, flowable composition lumen 7 is used for storage, e.g., of a medication and other flowable composition such as a liquid or a gel. In some embodiments, the flowable composition lumen is enclosed by a distal closed end of the syringe barrel, a lumen wall of the syringe barrel, and floating seal 3; that is, the flowable composition lumen occupies a distal portion of a syringe barrel lumen. In some embodiments, since floating seal 3 can move along in an axial direction, flowable composition lumen 7 is configured to have a variable volume, thus the fluid pressure inside flowable composition lumen 7 can change due to an axial movement of floating seal 3.

In some embodiments, using a medical puncturing device disclosed herein comprises applying pressure on actuation member 2, thereby advancing hollow puncture needle 6 forward in a distal direction, sequentially through floating seal 3 (e.g., by puncturing the floating seal or forcing open an existing aperture or slit through the floating seal) and through a distal closed end (e.g., by puncturing the distal closed end or forcing open an existing aperture or slit through the distal closed end) of the syringe barrel. The existing aperture or slit may be through the floating seal, e.g., from a proximal surface of the floating seal to a distal surface of the floating seal, thereby providing a through hole in the floating seal. The existing aperture or slit may be not through the entire floating seal, and advancing the needle distal end through the floating seal may comprise advancement through the existing aperture or slit and puncturing a portion of the floating seal in any suitable combination. For instance, the needle distal end may first advance through an existing aperture or slit from a proximal surface and then puncture the floating seal before emerging from a distal surface of the floating seal, or vice versa. In some embodiments, hollow puncture needle 6 pierces into an apparent or potential tissue void, cavity, or vessel, thereby placing needle distal opening 6a in the apparent or potential tissue void, cavity, or vessel. In some embodiments, needle body opening 6b is positioned inside flowable composition lumen 7, and floating seal 3 is elastically engaged with actuation member 2. In some embodiments, the fluid pressure in flowable composition lumen 7 is higher than the pressure inside the apparent or potential tissue void, cavity, or vessel.

At this time, the flowable composition inside flowable composition lumen 7 can flow through needle body opening 6b and needle distal opening 6a and into the apparent or potential tissue void, cavity, or vessel. In some embodiments, during an injection process, a user can simply maintain the pressure on actuation member 2, e.g., without further increasing the pressure. Under the action of the elastic engagement between floating seal 3 and actuation member 2, the flowable composition (e.g., a solution, a suspension, or a gel) inside flowable composition lumen 7 can enter needle body opening 6b and through the needle body passageway, thus achieving injection, penetration, and/or expansion of the apparent or potential tissue void, cavity, or vessel.

In some embodiments, before hollow puncture needle 6 pierces into an apparent or potential tissue void, cavity, or vessel, external pressure on needle distal opening 6a is higher than the fluid pressure in flowable composition lumen 7, e.g., due to the needle distal opening being in a tissue denser, harder, and/or less deformable than the apparent or potential tissue void, cavity, or vessel. Thus, the flowable composition inside the flowable composition lumen cannot exist needle distal opening 6a and into the surrounding tissue.

In some embodiments, by observing whether floating seal 3 moves forward due to the elastic engagement when actuation member 2 is held still under pressure, an operator can determine whether hollow puncture needle 6 has already pierced into an apparent or potential tissue void, cavity, or vessel, thereby informing the operator of the current needle depth and/or location of the needle distal opening and ensure accurate needle placement. In some embodiments, since the injection is controlled by fluid pressure changes in flowable composition lumen 7, the injection process does not require manually applying a force that is transmitted via relatively rigid medium (e.g., solid or liquid) in order to advance and precisely place the needle tip into an apparent or potential tissue void, cavity, or vessel. Rather, an abrupt force applied to actuation member 2 can be buffered due to the elastic engagement between actuation member 2 and floating seal 3, thus allowing more controllable and steady movement of the floating seal. In some embodiments, using a device disclosed herein, fluctuations in the flow speed can be prevented or reduced and steady injection can be achieved.

It should be noted that, the apparent or potential tissue gaps, voids, cavities, cavity systems, or vessels of the present disclosure can include but are not limited to an epidural space, a pleural cavity, a peritoneal cavity, an artery, a vein, a joint space (e.g., a knee join space), etc. Thus, the medical puncturing device disclosed herein also has the advantage of being highly versatile for use in any suitable apparent or potential tissue gaps, voids, cavities, cavity systems, or vessels. In some embodiments, the medical puncturing device can be used for epidural puncture and drug delivery (e.g., epidural anesthesia), pleural puncture and intrapleural drug delivery, peritoneal puncture and intraperitoneal drug delivery, or intraarticular injection. For example, applications include accessing suprachoroidal space (ocular), performing epidural injections (spinal cord access), accessing large vessels (arteries/veins) for inserting surgical wires (e.g., to access heart through vessels), accessing vessels for fistula access or catheter insertion, inserting through heart wall without damaging inner wall, accessing the abdomen (e.g. trocar access for minimally invasive surgery), injecting in fat under the skin, accessing insides of amniotic sac without damaging the fetus, performing a knee sac injection without damaging cartilage, injecting inside meninges without damaging brain tissue (drill in skull then use autostop on meninges), injecting between pericardium and heart, injecting between fascia and kidney, injecting between fibrous tissue layer and implants (for e.g. breast implant), injecting into other ocular spaces (e.g., for Deep Anterior Lamellar Keratoplasty (DALK) to separate epithelial cell layer from collagenous layer), or accessing collapsed lungs from outside. Also, the system may be used to deliver gene therapy including but not limited to viral vectors and/or transfected cells. In some embodiments, the flowable composition may include a variety of therapeutics. As non-limiting examples, therapeutics may include mRNA, CRISPR agents, RNAi, antibodies, nanobodies, nanoparticles, proteins, peptides, small molecules, aptamers, cells, extracellular vesicles, microRNA and the like.

In some embodiments, when hollow puncture needle 6 pierces through the syringe barrel distal closed end, the medical puncturing device can be in at least three states: a pre-puncture state, a surface tissue puncture state, and a fluidic communication state.

In some embodiments, in the pre-puncture state, the length range of hollow puncture needle 6 extending from the syringe barrel distal closed end is a pre-puncture length range.

Within this range, hollow puncture needle 6 has not yet started puncturing an organism or a tissue thereof.

In some embodiments, a system or device of the present disclosure comprises a flowable composition lumen pre-filled with a flowable composition. In some embodiments, prior to use of the system or device, the needle is already through the floating seal. In some embodiments, prior to use of the system or device, the needle is already through the floating seal and the syringe barrel distal end, e.g., a distal seal sealing the syringe barrel distal end.

In some embodiments, the flowable composition is of a relatively high viscosity, e.g., higher than water-like consistency, such as a gel or paste-like material. Elastic sleeve or sheath 4 shown in the figures of the present disclosure is optional, especially when the viscosity of the flowable composition is sufficient to prevent discharge from the needle body opening and/or needle distal opening when the openings are in the flowable composition lumen. For example, as shown in FIG. 3A, the needle can be through the floating seal such that needle body opening 6b is proximal to the floating seal while needle distal opening 6a is in the flowable composition lumen. Discharge of the flowable composition from the needle body opening can be prevented due to viscosity of the composition, and the elastic sheath is optional. Alternatively, as shown in FIG. 3B, the needle body opening 6b can be in the flowable composition lumen while needle distal opening 6a is outside the flowable composition lumen. Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be outside the flowable composition lumen, while needle body opening 6b can be proximal to the floating seal (e.g., as shown in FIGS. 3C, 6b1) or within the floating seal (e.g., as shown in FIGS. 3C, 6b2). Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be within a distal seal at the syringe barrel distal closed end (e.g., the needle distal opening can be blocked by the distal seal), while needle body opening 6b can be proximal to the floating seal (e.g., as shown in FIGS. 3D, 6b1), within the floating seal (e.g., as shown in FIGS. 3D, 6b2), or within the flowable composition lumen (e.g., as shown in FIGS. 3D, 6b3). Discharge of the flowable composition from the needle distal opening and the needle body opening can be prevented.

In some embodiments, for example prior to or during the use of the system or device, needle distal opening 6a can be within the flowable composition lumen, while needle body opening 6b can be within the floating seal (e.g., as shown in FIGS. 3E, 6b1) or within the flowable composition lumen (e.g., as shown in FIGS. 3E, 6b2). Discharge of the flowable composition from the needle body opening can be prevented.

In some embodiments, for example prior to or during the use of the system or device, needle distal opening 6a can be within the floating seal, while needle body opening 6b can be proximal to the floating seal (e.g., as shown in FIGS. 3F, 6b). Discharge of the flowable composition from the needle body opening can be prevented.

In some embodiments, in the surface tissue puncture state, the length range of hollow puncture needle 6 extending from the syringe barrel distal closed end is a surface tissue puncture length range. Within this range, the distal end of hollow puncture needle 6 has entered a tissue but has not yet entered the apparent or potential tissue void, cavity, or vessel (for example, not pierced into epidural space 14). In some embodiments, because the surface tissue is relatively dense, external pressure on needle distal opening 6a is higher than the fluid pressure in flowable composition lumen 7, therefore, no matter whether needle body opening 6b is connected to flowable composition lumen 7 or not, the flowable composition does not enter needle body opening 6b and/or exit needle distal opening 6a.

In some embodiments, while in the fluidic communication state, the length range of hollow puncture needle 6 extending from the syringe barrel distal closed end is the a fluidic communication. Within this range, the distal end of hollow puncture needle 6 has pierced into the apparent or potential tissue void, cavity, or vessel. In some embodiments, the device can be designed such that in the fluidic communication state, the fluid pressure in flowable composition lumen 7 is higher than the pressure inside the apparent or potential tissue void, cavity, or vessel. In some embodiments, in the fluidic communication state, needle body opening 6b has already positioned inside flowable composition lumen 7, and due to a difference in the internal (e.g., in the apparent or potential tissue void, cavity, or vessel) and external (e.g., in flowable composition lumen 7) pressures, the flowable composition inside lumen 7 can flow into the apparent or potential tissue void, cavity, or vessel through needle body opening 6b, the needle body passageway, and then needle distal opening 6a.

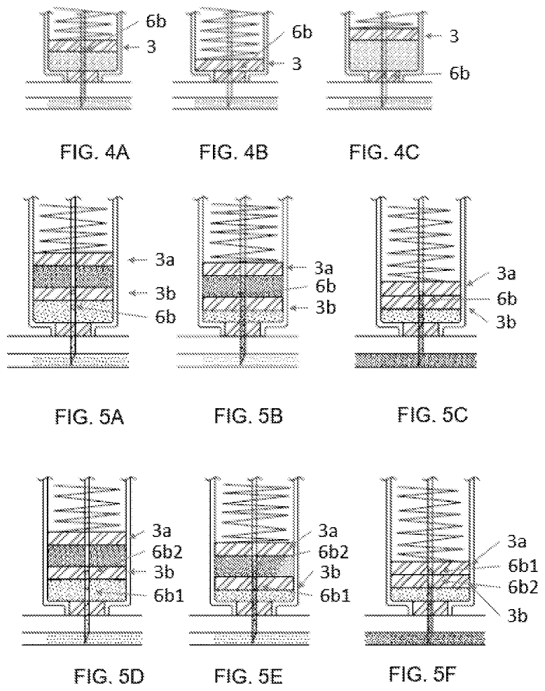

In some embodiments, floating seal 3 moves distally due to the elastic engagement with actuation member 2 (e.g., due to the pressure in the flowable composition lumen being higher than a backpressure at the needle distal opening in the apparent or potential tissue void, cavity, or vessel) until the floating seal seals needle body opening 6b (e.g., as shown in FIGS. 4A-4B). In some embodiments, the axial dimension of the needle body opening is no greater than the thickness of the floating seal. In some embodiments, the needle body opening can be completely sealed or blocked by the floating seal, at which time no more flowable composition exits needle distal opening 6a to enter the tissue void. In some embodiments, when the floating seal blocks the needle body opening, only a portion of the total volume of flowable composition has exited needle distal opening 6a (e.g., as shown in FIG. 4A). In some embodiments, when the floating seal blocks the needle body opening, the total volume of flowable composition in the lumen has exited needle distal opening 6a (e.g., as shown in FIG. 4B).

In some embodiments, when the needle body opening can be in the distal seal or in a tissue of a subject, the flowable composition will stop existing needle distal opening 6a (e.g., as shown in FIG. 4C). In some embodiments, the distance between needle distal opening 6a and needle body opening 6b can be keep constant. In some embodiments, the distance between needle distal opening 6a and needle body opening 6b can be varied. For example, a needle having a suitable distance between needle distal opening 6a and needle body opening 6b can be selected based on a known or estimated depth of the tissue to be accessed. In some embodiments, stopper 1a is provided inside the syringe lumen and can be used to limit the forward movement of floating seal 3 in order to achieve precise injection, for example, injection of a pre-determined volume.

In some embodiments, once floating seal 3 contacts stopper 1a, further distal movement of the floating seal is limited, thereby stabilizing floating seal 3 for subsequent operation.

In some embodiments, a system or device disclosed herein comprises two or more floating seals. For example, as shown in FIG. 5A, a first lumen is formed between floating seal 3b and the distal seal of the syringe barrel, and a second lumen is formed between floating seal 3a and floating seal 3b. In some embodiments, the first lumen and the second lumen comprise the same flowable material. In some embodiments, the first lumen and the second lumen comprise different flowable compositions. In some embodiments, the first lumen and the second lumen comprise the same medicament (e.g., active pharmaceutical ingredient) in the same or different flowable carriers or excipients. In some embodiments, the first lumen and the second lumen comprise different medicaments (e.g., active pharmaceutical ingredients) in the same or different flowable carriers or excipients. In some embodiments, the first lumen comprises a medicament and the second lumen comprises a pharmaceutically acceptable carrier or excipient such as a saline, or vice versa.

In some embodiments, the flowable compositions in the first lumen and the second lumen can be sequentially delivered to an apparent or potential tissue void, cavity, or vessel. In some embodiments, the flowable compositions in the first lumen and the second lumen can be mixed in the apparent or potential tissue void, cavity, or vessel. In some embodiments, the flowable composition in the first lumen enters the apparent or potential tissue void, cavity, or vessel in order to access and/or expand the tissue void, cavity, or vessel. Subsequently, the flowable composition in the second lumen comprising a medicament can enter the apparent or potential tissue void, cavity, or vessel. For example, as shown in FIG. 5A, when needle distal opening 6a is in the apparent or potential tissue void, cavity, or vessel while needle body opening 6b is in the first lumen (between floating seal 3b and the distal seal of the syringe barrel), the flowable composition in the first lumen is delivered to the tissue. In FIG. 5B, needle distal opening 6a can be held still in the apparent or potential tissue void, cavity, or vessel, when floating seal 3b moves distally and needle body opening 6b contacts the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b, as shown in FIG. 5C. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, the dimension of needle body opening 6b along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the dimension of needle body opening 6b along the needle axis is no greater than the thickness of floating seal 3a and floating seal 3b combined. In some embodiments, the dimension of needle body opening 6b along the needle axis is greater than the thickness of floating seal 3b and less than the thickness of floating seal 3a and floating seal 3b combined. In some embodiments, a system or device disclosed herein comprises one or more additional floating seals (e.g., a third floating seal, 3c) that are proximal to floating seal 3a, distal to floating seal 3b, and/or between floating seal 3a and floating seal 3b, such that a third flowable composition may be delivered before the first flowable composition, after the second flowable composition, or between the first and second flowable compositions.

In some embodiments, a system or device disclosed herein comprises two or more needle body openings. In some embodiments, a system or device disclosed herein comprises two or more needle body openings and two or more floating seals. For example, as shown in FIG. 5D, when needle distal opening 6a is in the apparent or potential tissue void, cavity, or vessel while needle body opening 6b1 is in the first lumen (between floating seal 3b and the distal seal of the syringe barrel) and needle body opening 6b2 is blocked by floating seal 3b, the flowable composition in the first lumen is delivered to the tissue. In FIG. 5E, needle distal opening 6a can be held still in the apparent or potential tissue void, cavity, or vessel, when floating seal 3b moves distally to block needle body opening 6b1, allowing needle body opening 6b2 to contact the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b2 (and/or needle body opening 6b1) as shown in FIG. 5F. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, the distance between needle body opening 6b1 and needle body opening 6b2 along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the distance between needle body opening 6b1 and needle body opening 6b2 along the needle axis is no greater than the thickness of floating seal 3a and floating seal 3b combined. In some embodiments, the distance between needle body opening 6b1 and needle body opening 6b2 along the needle axis is greater than the thickness of floating seal 3b and less than the thickness of floating seal 3a and floating seal 3b combined. In some embodiments, a system or device disclosed herein comprises one or more additional needle body openings (e.g., a third needle body opening, 6b3) that are proximal to needle body opening 6b2, distal to needle body opening 6b1, and/or between needle body openings 6b1 and 6b2, such that a third flowable composition may be delivered before the first flowable composition, after the second flowable composition, or between the first and second flowable compositions.

Described below are multiple embodiments to control the termination of the injection process using a medical puncturing device disclosed herein.

In some embodiments, when the medical puncturing device is in a fluidic communication state, floating seal 3 moves forward due to the elastic engagement with actuation member 2 until it seals needle body opening 6b. Once needle body opening 6b is sealed, the injection process is terminated. In some embodiments, the axial position of needle body opening 6b within the flowable composition lumen 7 limits the maximum injection volume of the medical puncturing device. In some embodiments, when needle body opening 6b is blocked or sealed by floating seal 3, floating seal 3 has not contacted a wall at the syringe barrel distal closed end. In some embodiments, flowable composition lumen 7 is not completely emptied and there is still flowable composition between floating seal 3 and the wall at the syringe barrel distal closed end.

In some embodiments, when flowable composition lumen 7 needs to be emptied, floating seal 3 can be designed to seal needle body opening 6b when the floating seal contacts the syringe barrel distal closed end. In some embodiments, needle body opening 6b is at the distal end of flowable composition lumen 7. In some embodiments, floating seal 3 contacts a wall at the syringe barrel distal closed end and needle body opening 6b is blocked or sealed by floating seal 3 and/or the wall at the syringe barrel distal closed end. In some embodiments, flowable composition lumen 7 is emptied and there is no or little flowable composition between floating seal 3 and the wall at the syringe barrel distal closed end.

In some embodiments, as the flowable composition inside flowable composition lumen 7 gradually enters the apparent or potential tissue void, cavity, or vessel, there can be a state wherein the fluid pressure inside flowable composition lumen 7 reaches equilibrium with the pressure in the apparent or potential tissue void, cavity, or vessel. At this time, floating seal 3 no longer moves, due to the balance of forces. In order to continue injection and/or empty flowable composition lumen 7, additional force is needed on floating seal 3 in order to move it forward toward the syringe barrel distal closed end.

For example, as shown in FIGS. 2A-2E, one, two, or more axially extending sliding grooves (not shown) can be provided on a body wall of syringe barrel 1. A slider matching a sliding groove can be provided on actuation member 2 (e.g., a slider can comprise a portion of actuation member 2 extending outside of syringe barrel 1), thus increasing the upper limit of the movement distance or stroke of actuation member 2 since the movement is not limited by the proximal end of actuation member 2. When floating seal 3 can no longer move due to the equilibrium of forces (e.g., between pressure inside flowable composition lumen 7 and the apparent or potential tissue void, cavity, or vessel), more pressure can be applied on a slider of actuation member 2 to drive actuation member 2 forward distally, which in turn can increase the elastic resilience between floating seal 3 and actuation member 2, thus breaking the force equilibrium and moving floating seal 3 forward toward the distal end of the syringe barrel. This way, more flowable composition can be expelled from flowable composition lumen 7, in some embodiments emptying flowable composition lumen 7.

In some embodiments, other drive structures can be used to move floating seal 3 further until it contacts a wall of the syringe barrel distal closed end. Exemplary drive structures are described below.

In some embodiments, an axially extending sliding groove can be provided on a peripheral wall of syringe barrel 1, proximal to floating sealing 3. In some embodiments, a manual control part can include an actuation member 2′ (which may be in the form of a slider) that is slidingly matched with the sliding groove of the peripheral wall of the syringe barrel. In some embodiments, a portion of actuation member (e.g., slider) 2′ extends outside of the syringe barrel through the sliding groove, which is convenient for a user to manipulate. In some embodiments, floating sealing 3 and actuation member (e.g., slider) 2′ form an elastic connection. For example, floating sealing 3 and actuation member (e.g., slider) 2′ can engage each other via elastic piece (e.g., spring) 4′ as shown in Step 1, FIG. 2G, whereas floating sealing 3 and actuation member (e.g., slider) 2 can engage each other via elastic piece (e.g., spring) 4. In some embodiments, actuation member 2 may comprise a rod that is configured to insert through a space between portions of actuation member 2′ such that actuation members 2 and 2′ do not interfere with each other. In some embodiments, elastic piece (e.g., spring) 4 and elastic piece (e.g., spring) 4′ may function independently and do not interfere with each other. In some embodiments, spring 4 is smaller than spring 4′, for instance, the average diameter of spring 4 can be can be smaller than the average diameter of spring 4′. In some embodiments, elastic piece 4′ is nested inside elastic piece 4. In Step 2, FIG. 2G, a force can be applied to actuation member 2 to move the needle distally while maintaining the position of floating sealing 3. In some embodiments, as shown in Step 3, FIG. 2G, a force can be applied on actuation member 2′ to move it distally along the axial direction of the sliding groove on the peripheral wall of the syringe barrel. This way, elastic piece (e.g., spring) 4′ between floating sealing 3 and actuation member (e.g., slider) 2′ can be elastically compressed. In some embodiments, when the position of actuation member (e.g., slider) 2′ is maintained, under the action of an elastic force, floating sealing 3 can break the equilibrium of forces and continue to move distally until the volume of discharged flowable composition reaches a target volume. In some embodiments, actuation member (e.g., slider) 2′ can be moved distally as shown in Step 4, FIG. 2G, to move floating sealing 3 further distally in order to discharge the flowable composition from the needle.

In some embodiments, the medical puncturing device comprises an element configured for an operator to manually control movement of the floating seal using one or both hands. In some embodiment, the manual control element can be moved using one or more fingers, for example, one finger of the same hand holding the syringe barrel. In some embodiments, the manual control element is fixed to floating seal 3 and partially extends outside the syringe barrel. In some embodiments, when the flowable composition volume injected into the apparent or potential tissue void, cavity, or vessel does not reach a target volume, while floating seal 3 is no longer moving due to the equilibrium of forces, the operator can drive further movement of floating seal 3 forward by moving the portion of the manual control element that extends outside the syringe barrel, until the expelled flowable composition volume reaches the target volume. In some embodiments, using the manual control element helps empty flowable composition lumen 7. These embodiments are not limited to situations where flowable composition lumen 7 needs to be emptied.

In some embodiments, the medical puncturing device can achieve delivery (e.g., via injection) of a flowable composition of a defined volume with precision, and/or the ability to control the volume to be delivered. In some embodiments, the defined volume is a preset volume prior to the delivery. In some embodiments, the defined volume is one of multiple volumes that an operator can select during the delivery, and the delivered volume may be different from a preset volume. In some embodiments, as shown in FIGS. 1A-1F, FIGS. 2A-2F, and FIG. 11, axial stopper 1a is provided inside the syringe lumen and distal to floating seal 3, and is used to limit the forward movement of floating seal 3. In some embodiments, when the medical puncturing device is in the fluidic communication state, needle body opening 6b can be distal to axial stopper 1a, and floating seal 3 can move forward due to the elastic engagement with actuation member 2.

In some embodiments, floating seal 3 is moved to the position limited by axial stopper 1a. In some embodiments, when floating seal 3 moves to the position limited by axial stopper 1a, pressure in flowable composition lumen 7 is still no less than the pressure inside the apparent or potential tissue void, cavity, or vessel. In some embodiments, floating seal 3 can be pushed forward to the position limited by axial stopper 1a by the elastic resilience between floating seal 3 and actuation member 2, and there is no need to rely on additional driving structure or force to move floating seal 3 to the position limited by axial stopper 1a.

In some embodiments, before floating seal 3 is moved to the position limited by axial stopper 1a by the elastic resilience between the floating seal and actuation member 2, pressure in flowable composition lumen 7 has already become equal with the pressure inside the apparent or potential tissue void, cavity, or vessel (that is, due to balance of forces, floating seal 3 is no longer moving before it reaches axial stopper 1a). At this time, just by the elastic resilience between floating seal 3 and actuation member 2, floating seal 3 is not pushed forward to the position limited by axial stopper 1a. Thus, in some embodiments, one or more additional driving structure or mechanism can be employed to further push forward floating seal 3. For example, the additional driving structure or mechanism can comprise a manual control element described herein (e.g., as shown in FIGS. 2A-2E). In some embodiments, axial stopper 1a provides a mechanism for achieving fluid injection of set volumes.

Described below are multiple embodiments for puncture and injection timing of a medical puncturing device disclosed herein.

In some embodiments, when the medical puncturing device is in pre-puncture state, that is, when the length of hollow puncture needle 6 extending from the syringe barrel distal closed end is within the pre-puncture length range (or when hollow puncture needle 6 has already pierced the syringe barrel distal closed end but has not yet started puncturing the organism or a tissue thereof), needle body opening 6b remains above (e.g., proximal to) flowable composition lumen 7. When provided in this way, early leakage from needle distal opening 6a can be prevented and the reliability of the medical puncturing device can be improved.

In some embodiments, corresponding structure(s) can be provided on the device to prevent early leakage before hollow puncture needle 6 punctures the tissue and/or before needle distal opening 6a reaches the apparent or potential tissue void, cavity, or vessel. For example, axially extending circular contacting element 1b can be formed at the syringe barrel distal closed end. In some embodiments, the axial length of circular contacting element 1b is set to be the same as the difference between the upper and lower limits of the pre-puncture length range of hollow puncture needle 6 (that is, the difference in needle pre-puncture lengths between when hollow puncture needle 6 pierces the syringe barrel distal closed end and when it starts puncturing the organism or tissue). Under this setting, as long as the distal end of hollow puncture needle 6 is still within the axial length range of circular contacting element 1b, early leakage will not happen at needle distal opening 6a. When puncturing, circular contacting element 1b can come into contact with the surface of the organism or tissue first to stabilize the medical puncturing device. Then, pressure can be applied to actuation member 2 to start the puncture operation.

In some embodiments, when the medical puncturing device is in the surface tissue puncture state, that is, when the length of hollow puncture needle 6 extending from the syringe barrel distal closed end is within the surface tissue puncture length range (or when the distal end of hollow puncture needle 6 has pierced the surface tissue but has not yet entered the apparent or potential tissue void, cavity, or vessel), needle body opening 6b is at least partially connected to flowable composition lumen 7. In some embodiments, before the distal end of hollow puncture needle 6 pierces into the apparent or potential tissue void, cavity, or vessel, fluidic communication among flowable composition lumen 7, needle distal opening 6a and needle body opening 6b is established. In some embodiments, the flowable composition in lumen 7 can enter the needle body passageway (via needle body opening 6b) of hollow puncture needle 6 in advance, removing at least part of the air that may be in the needle body passageway, thereby reducing the amount of air entering the apparent or potential tissue void, cavity, or vessel.

In some embodiments, when the distal end of hollow puncture needle 6 starts to pierce into the surface tissue, needle body opening 6b starts to connect with flowable composition lumen 7. In some embodiments, when the distal end of hollow puncture needle 6 pierces into the apparent or potential tissue void, cavity, or vessel, the needle body passageway of hollow puncture needle 6 has already been filled with the flowable composition, thereby eliminating or reducing the possibility of air entering the apparent or potential tissue void, cavity, or vessel.

In some embodiments, when the medical puncturing device is in the fluidic communication state, that is, when the length of hollow puncture needle 6 extending from the syringe barrel distal closed end is within the fluidic communication length range (or when the distal end of hollow puncture needle 6 has pierced into the apparent or potential tissue void, cavity, or vessel), needle body opening 6b has been positioned inside flowable composition lumen 7, achieving maximum flow at needle body opening 6b and thereby increasing injection speed.

The embodiments described herein can be implemented separately or in any suitable combination.

In some embodiments, a device disclosed herein can prevent fluid backflow and/or reverse spill through needle body opening 6b.

In some embodiments, there is a risk for fluid backflow and/or reverse spill from needle body opening 6b when needle distal opening 6a is connected with flowable composition lumen 7, while needle body opening 6b is still at the proximal end of floating seal 3. In some embodiments, there is a risk for fluid backflow and/or reverse spill from needle body opening 6b when needle distal opening 6a is inside the apparent or potential tissue void, cavity, or vessel, while needle body opening 6b is still at the proximal end of floating seal 3. In some embodiments, an elastic sheath 4 covering the outside of hollow puncture needle 6 can be provided within the actuation unit (e.g., elastic movement unit), e.g., between the needle base and floating seal 3. In some embodiments, when needle body opening 6b is at the proximal end of floating seal 3 (e.g., when needle body opening 6b is not connected to flowable composition lumen 7), elastic sheath 4 can keep the needle body opening 6b sealed, thereby effectively avoiding backflow and/or reverse spill of the flowable composition, preventing contamination of the area proximal to floating seal 3, reducing fluid loss, and improving product reliability.

In some embodiments, elastic sheath 4 is not used to seal needle body opening 6b, but simply as an elastic engagement part between floating seal 3 and actuation member 2. In some embodiments, by moving actuation member 2 forward, elastic sheath 4 between floating seal 3 and actuation member 2 can become compressed, thereby forming elastic resilience between floating seal 3 and actuation member 2, which can in turn drive floating seal 3 forward. In some embodiments, the elastic engagement part between floating seal 3 and actuation member 2 can comprise or be a spring 5, which is attached to floating seal 3 and actuation member 2 at its two axial ends, respectively. The attachment at either or both ends of the spring can be direct or indirect. The attachment at either or both ends of the spring can be releasable or not releasable. The spring, the floating seal, and the actuation member (e.g., pressing element) can be separately manufactured and then assembled in any suitable order. Alternatively, any two or more of the spring, the floating seal, and the actuation member (e.g., pressing element) can be integral, e.g., made as one piece. Spring 5 and elastic sheath 4 can be implemented separately or in combination.

In some embodiments, the elastic engagement between floating seal 3 and actuation member 2 can be achieved through other methods besides providing one or more elastic engagement parts. For example, floating seal 3 and actuation member 2 can be provided as a one-piece integrated actuation unit (e.g., elastic movement unit).

In some embodiments, provided herein are devices and methods for implantation into apparent or potential tissue gaps, cavity systems, and vessels using a medical puncturing device disclosed herein. For ease of understanding, a catheter is used as an example for the implanted medical device. In some embodiments, a method disclosed herein comprises using a catheter guiding structure for guiding catheter 11 into the needle body passageway of hollow puncture needle 6. In some embodiments, a catheter guiding structure is provided in a medical puncturing device disclosed herein.

In some embodiments, as shown in FIGS. 6-8, the catheter guiding structure comprises an angled guiding groove 3a, which is provided in or engages floating seal 3 and extends towards hollow puncture needle 6 at an angle. In some embodiments, when flowable composition lumen 7, needle body opening 6b, and needle distal opening 6a are connected, a flowable composition can enter and expand the apparent or potential tissue void, cavity, or vessel. In some embodiments, catheter 11 can be implanted through angled guiding groove 3a, needle body opening 6b, the needle body passageway of hollow puncture needle 6, and needle distal opening 6a into the expanded apparent or potential tissue void, cavity, or vessel.

It should be noted that, angled guiding groove 3a can be provided as a groove through floating seal 3 in a proximal/distal direction, or as a non-through groove formed on a proximal surface of floating seal 3.

In some embodiments, angled guiding groove 3a is a through groove. In some embodiments, the catheter guiding structure further comprises valve 9 provided in or engages angled guiding groove 3a, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into needle body opening 6b through the opened valve. In some embodiments, the catheter guiding structure further comprises a guiding groove plug configured to be removably inserted in angled guiding groove 3a, and the guiding groove plug can be pulled out when catheter 11 needs to be implanted.

In some embodiments, angled guiding groove 3a is a non-through groove. In some embodiments, the angled guiding groove is punctured directly by catheter 11 to be implanted. In some embodiments, the angled guiding groove is punctured by a piercing component other than the catheter, and catheter 11 can be threaded through the punctured opening into needle body opening 6b.

In some embodiments, to match the guiding direction of angled guiding groove 3a, needle body opening 6b can be provided as an angled opening, which opens obliquely backwards, so that needle body opening 6b can align with angled guiding groove 3a, thereby precisely guiding catheter 11 through the angled guiding groove and into the needle body opening.

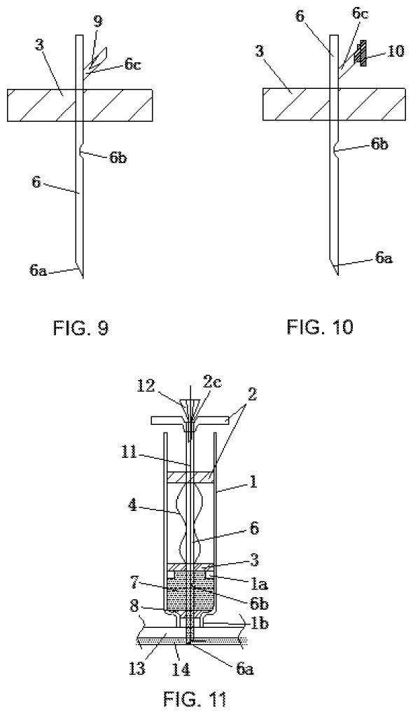

In some embodiments, for example as shown in FIG. 9 and FIG. 10, the catheter guiding structure comprises an angled guiding needle hole 6c which is formed or provided on the body wall of hollow puncture needle 6 and opens obliquely backwards. In some embodiments, angled guiding needle hole 6c remains proximal to floating seal 3, for example, when the medical puncturing device is in a fluidic communication state. In some embodiments, catheter 11 can be threaded into the needle body passageway of hollow puncture needle 6 through angled guiding needle hole 6c. In some embodiments, catheter 11 can be implanted into an apparent or potential tissue void, cavity, or vessel (or an apparent or potential tissue void, cavity, or vessel that has been expanded with a flowable composition) through needle distal opening 6a.

In some embodiments, the catheter guiding structure can further comprise valve 9 provided in or engages angled guiding needle hole 6c, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into a needle body passageway (which may be connected to or separate from the needle body passageway connecting needle body opening 6b and needle distal opening 6a) through the opened valve and angled guiding needle hole 6c. In some embodiments, the catheter guiding structure can further comprise needle hole plug 10 configured to be removably inserted in angled guiding needle hole 6c, and needle hole plug 10 can be pulled out for the implantation operation of catheter 11 to begin. In some embodiments, guiding needle hole 6c is connected needle distal opening 6a. The needle body passageway connecting needle distal opening 6a and needle body opening 6b can be the same as or separate from the needle body passageway connecting needle distal opening 6a and guiding needle hole 6c. In some embodiments, guiding needle hole 6c is connected to a needle distal opening other than needle distal opening 6a connected to needle body opening 6b. The needle body passageway connecting needle body opening 6b to a needle distal end can be completely separate from the needle body passageway connecting guiding needle hole 6c to a needle distal end. The needle body passageway connecting needle body opening 6b to a needle distal end can be at least partially overlapping or in fluidic communication with the needle body passageway connecting guiding needle hole 6c to a needle distal end.

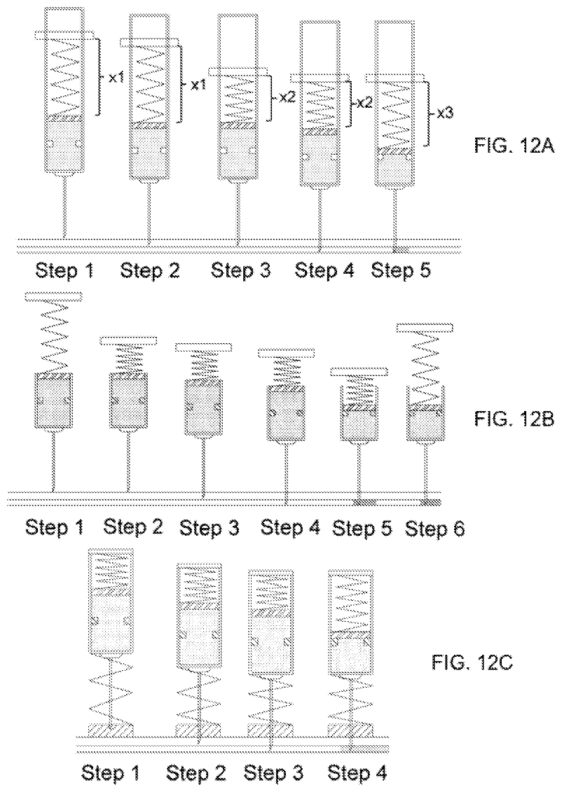

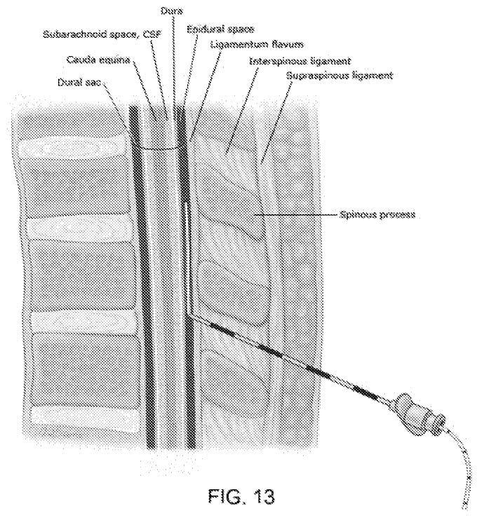

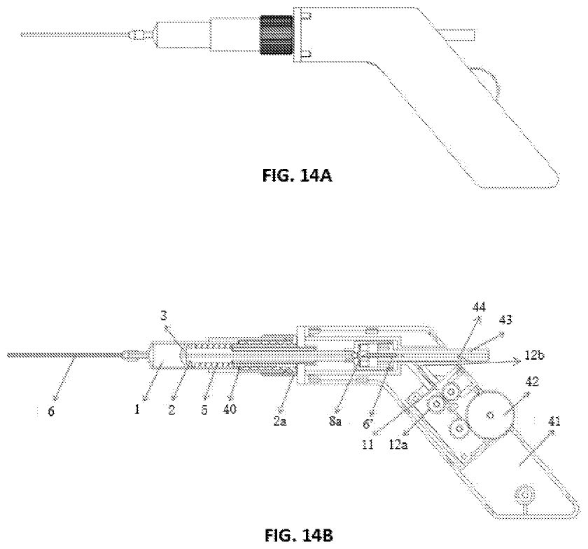

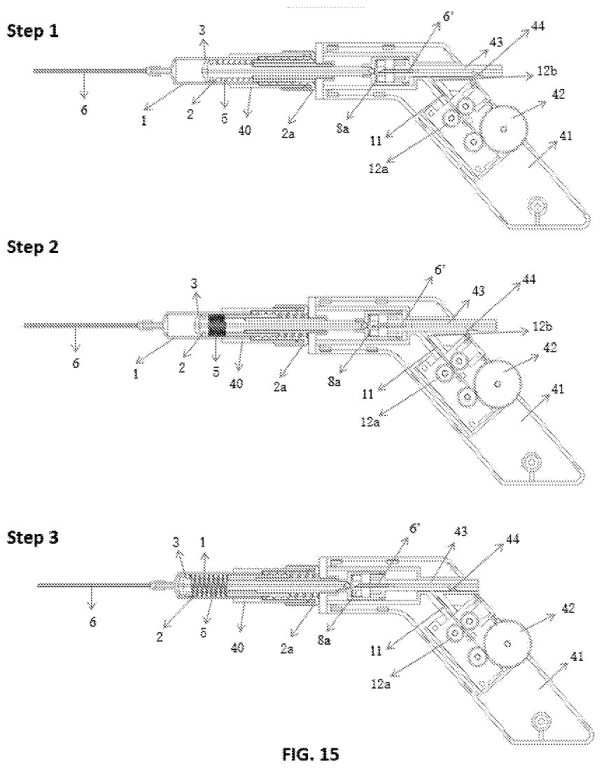

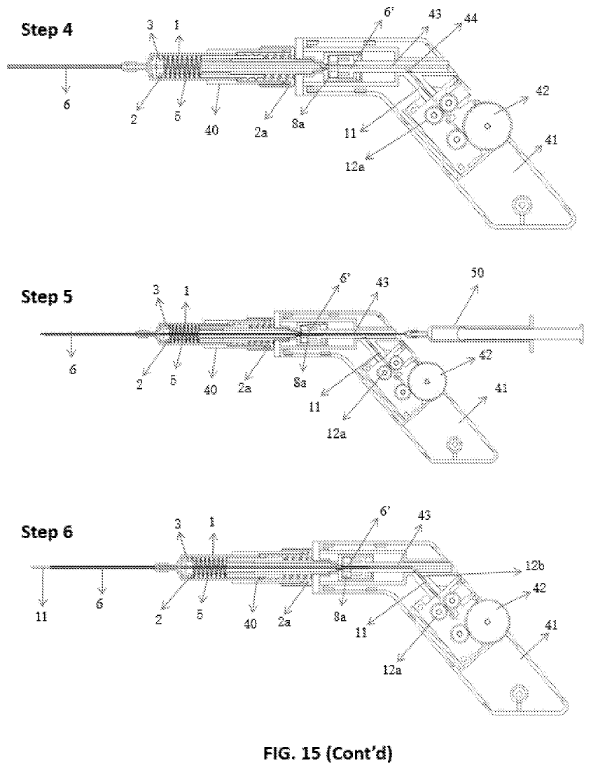

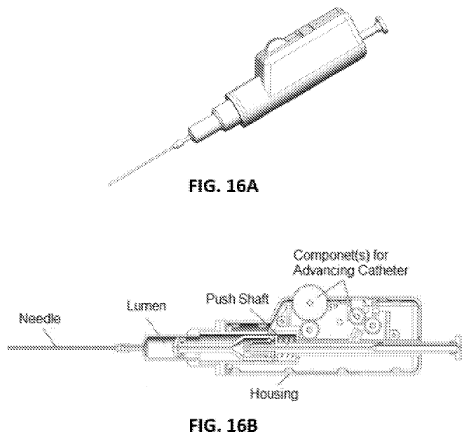

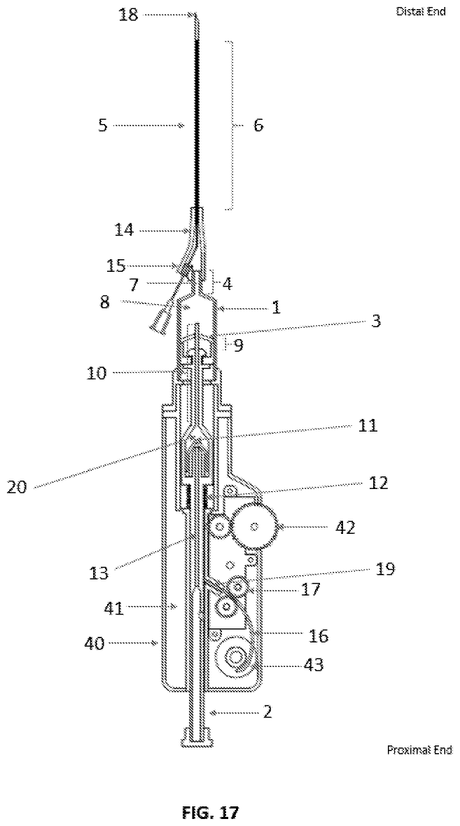

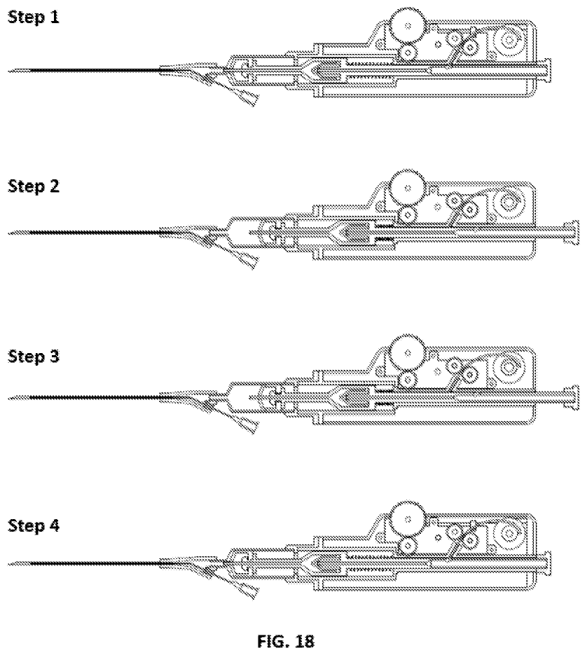

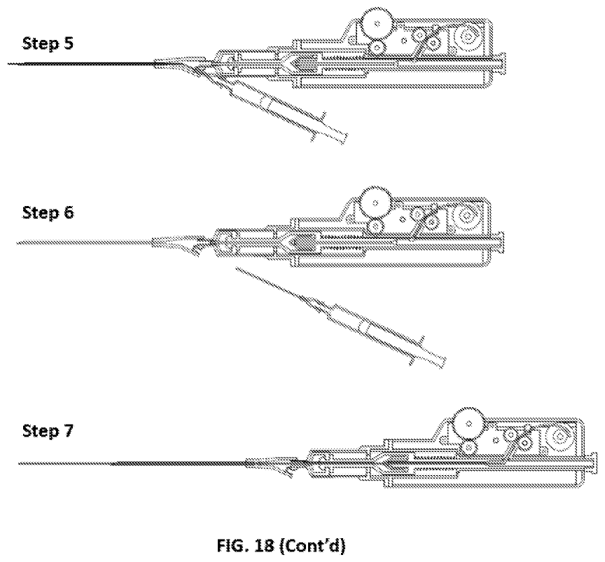

In some embodiments, for example as shown in FIG. 11, the catheter guiding structure comprises a central guiding groove 2c that is formed or provided on a proximal surface of actuation member 2. In some embodiments, central guiding groove 2c comprises an aperture or can form an aperture in the center of proximal surface of actuation member 2. In some embodiments, central guiding groove 2c can be punctured to provide an aperture. In some embodiments, a needle proximal opening is provided on hollow puncture needle 6 and is aligned with central guiding groove 2c along the axis. In some embodiments, when catheter 11 needs to be implanted, central guiding groove 2c can be punctured and catheter 11 can be threaded into a needle body passageway (which may be connected to or separate from the needle body passageway connecting needle body opening 6b and needle distal opening 6a) through the punctured opening of central guiding groove 2c and the needle proximal opening of hollow puncture needle 6. In some embodiments, catheter 11 can be implanted into an apparent or potential tissue void, cavity, or vessel (or an apparent or potential tissue void, cavity, or vessel that has been expanded with a flowable composition) through a needle distal opening, such as needle distal opening 6a or a different needle distal opening.

In some embodiments, disclosed herein is a kit comprising components configured to be assembled to form a medical puncturing device disclosed herein.

In some embodiments, the kit for assembling a medical puncturing device comprises a puncture control module and a flowable composition storage module (e.g., a fluid storage module). In some embodiments, the puncture control module and the flowable composition storage module are independently manufactured and/or provided. In some embodiments, the puncture control module comprises a first syringe unit, as well as an actuation unit (e.g., elastic movement unit), and hollow puncture needle 6, which are provided inside a syringe barrel of the first syringe unit. It can be seen based on the embodiments disclosed herein that the puncture control module can further comprise other parts or components, such as elastic sheath 4 and spring 5. In some embodiments, the fluid storage module comprises a second syringe unit, flowable composition lumen 7 which is formed inside a syringe barrel of the second syringe unit, and a module packaging component which is removably provided at the proximal end of the second syringe unit. In some embodiments, a removable connection structure is formed between the first syringe unit and the second syringe unit. In some embodiments, the first syringe unit and the second syringe unit form syringe barrel 1 after being connected with each other. It can be seen based on the embodiments disclosed herein that the fluid storage module can further comprise other parts such as distal seal 8.

In some embodiments, the puncture control module and the fluid storage module can be manufactured, assembled, and/or packaged separately, and then assembled with each other and optionally with other modules, components, and/or parts into the medical puncturing device disclosed herein. In some embodiments, the module packaging component is used to seal the proximal end of flowable composition lumen 7. In some embodiments, when assembling the puncture control module and the fluid storage module, the module packaging component can be removed.

In some embodiments, provided herein is a medical apparatus assembly and a system comprising the same. As shown in FIG. 7 and FIG. 11, in some embodiments the medical apparatus assembly comprises catheter 11 and the medical puncturing device comprising the catheter guiding structure disclosed herein. In some embodiments, catheter 11 can be implanted into an apparent or potential tissue void, cavity, or vessel by the medical puncturing device. The medical apparatus assembly described herein can have all of the technical effects provided by the medical puncturing device.

In some embodiments, the medical apparatus assembly comprises hollow auxiliary guiding needle 12, which is matched to be used with the catheter guiding structure. In some embodiments, the needle body passageway diameter of auxiliary guiding needle 12 is large enough to accommodate catheter 11 and allow the catheter to thread in. In some embodiments, during an operation to implant catheter 11, auxiliary guiding needle 12 is connected to the catheter guiding structure so that catheter 11 can sequentially go through the needle body passageway of auxiliary guiding needle 12, the catheter guiding structure, the needle body passageway of hollow puncture needle 6, and then into an apparent or potential tissue void, cavity, or vessel through needle distal opening 6a. In some embodiment, the apparent or potential tissue void, cavity, or vessel is expanded with a flowable composition using a medical puncturing device disclosed herein, prior to the implant of the catheter. In some embodiment, the catheter is implanted as the apparent or potential tissue void, cavity, or vessel is being expanded with a flowable composition using a medical puncturing device disclosed herein. In some embodiment, the catheter is implanted prior to the apparent or potential tissue void, cavity, or vessel being expanded with a flowable composition using a medical puncturing device disclosed herein.

In some embodiments, as shown in FIG. 7, the catheter guiding structure comprises through angled guiding groove 3a and one-way valve 9, which is embedded in angled guiding groove 3a and can be opened and closed. In some embodiments, needle body opening 6b is provided as an angled opening which opens obliquely backwards. In some embodiments, when implanting catheter 11, auxiliary guiding needle 12 is used to open one-way valve 9 so that the auxiliary guiding needle can be positioned inside angled guiding groove 3a. In some embodiments, the distal end of auxiliary guiding needle 12 advances into needle body opening 6b, and catheter 11 can sequentially advance through the needle body passageway of auxiliary guiding needle 12, the needle body passageway of hollow puncture needle 6, and the needle distal opening 6a and then be implanted into an apparent or potential tissue void, cavity, or vessel.

In some embodiments, as shown in FIG. 11, the catheter guiding structure comprises a central guiding groove 2c. In some embodiments, a needle proximal opening is formed on hollow puncture needle 6, which is aligned with central guiding groove 2c along its axis. In some embodiments, when implanting catheter 11, central guiding groove 2c can be punctured by auxiliary guiding needle 12, such that auxiliary guiding needle 12 is axially aligned with the proximal opening of hollow puncture needle 6. In some embodiments, catheter 11 is threaded into a needle body passageway of hollow puncture needle 6 by sequentially advancing through a needle body passageway of auxiliary guiding needle 12, and a proximal opening of hollow puncture needle 6, and is then implanted into an apparent or potential tissue void, cavity, or vessel through a needle distal opening such as needle distal opening 6a.

In some embodiments, the pressing shaft comprises a threaded portion configured to be in threaded engagement with a control knob. For example, the control knob can comprises an internal helical thread configured to engage a threaded portion of the pressing shaft. In some embodiments, the control knob can be rotated along a central axis, and through the threaded engagement, rotation of the control knob can drive translation of the pressing shaft in an axial direction. In some embodiments, the pressing shaft is moved along a helical path having a rotatory component and a translational component in an axial direction relative to the housing (or shell). In some embodiments, depending on whether the control knob is rotated clockwise or counterclockwise, the translational movement of the pressing shaft can be in a distal direction (e.g., towards an epidural space of a subject) or in a proximal direction (e.g., towards an operator). In some examples, clockwise rotation of the control knob advances the pressing shaft in a distal direction, whereas counterclockwise rotation of the control knob retracts the pressing shaft in a proximal direction. In other examples, counterclockwise rotation of the control knob advances the pressing shaft in a distal direction, whereas clockwise rotation of the control knob retracts the pressing shaft in a proximal direction.

In some embodiments, the pressing shaft is coupled to the syringe needle such that movement of the pressing shaft in an axial direction can lead to and/or allow movement of the syringe needle. In some embodiments, the pressing shaft and the syringe needle are directly coupled. In some embodiments, the pressing shaft and the syringe needle are indirectly coupled. In some embodiments, the pressing shaft and the syringe needle are elastically coupled. In some embodiments, the pressing shaft and the syringe needle elastically engage each other. In some embodiments, the pressing shaft and the syringe needle are coupled via an elastic connection. In some embodiments, the pressing shaft and the syringe needle are fixedly or removably coupled. In some embodiments, the pressing shaft and the syringe needle fixedly or removably engage each other. In some embodiments, the pressing shaft and the syringe needle are coupled via a fixed connection. In some embodiments, the connection between the pressing shaft and the syringe needle is sufficiently rigid such that the pressing shaft can drive advancement or retraction of the syringe needle. In some embodiments, the syringe needle is provided on a needle base or seat that is part of the pressing shaft or that is directly or indirectly coupled to the pressing shaft. In some embodiments, the needle base or seat is elongated axially and has a smaller cross-sectional area than the cross-sectional area of a portion of the pressing shaft that directly abuts the needle base or seat. In some embodiments, the needle base or seat is fixedly coupled to the pressing shaft. In some embodiments, the needle base or seat is integral to the pressing shaft. In some embodiments, the pressing shaft and the syringe needle are coupled via a needle base or seat that is sufficiently rigid in at least an axial direction, such that the pressing shaft can be moved axially distally or proximally in order to advance or retract the syringe needle relative to the housing or shell.

In some embodiments, the pressing shaft (e.g., pressing shaft 2 in FIG. 12) and the piston rod (e.g., push rod 15 in FIG. 12) are coupled via an elastic element or piece, such as a spring (e.g., spring 5 in FIG. 12). In some embodiments, the elastic element or piece is directly or indirectly coupled to the pressing shaft and/or the needle base or seat, or a portion thereof. For example, a portion (e.g., a proximal end) of the elastic element or piece can directly or indirectly engage a portion of the pressing shaft or needle base or seat. The elastic element or piece can fixedly or removably engage a proximal portion of the needle base or seat. In some embodiments, the elastic element or piece is directly or indirectly coupled to the piston rod. For example, a portion (e.g., a distal end) of the elastic element or piece can directly or indirectly engage a portion (e.g., a proximal end) of the piston rod. In some embodiments, the elastic element or piece is fixedly or removably coupled to the piston rod. In some embodiments, the pressing shaft can be pushed distally relative to the housing in order to exert a force on the elastic element or piece (e.g., spring), which in turn exerts a force on the piston rod, while the syringe needle is advanced distally by the pressing shaft.

In some embodiments, the needle base or seat or a portion thereof is elongated axially, providing space between a portion of the pressing shaft and the piston rod configured to accommodate one or more elastic elements or pieces. In embodiments where multiple elastic elements or pieces are used, any two or more of the elastic elements or pieces can be arranged in tandem or in parallel. Each elastic element or piece can be in the form of a flexible sheath or tube, a spring, an annular ring, an elongated rod or stripe, or any combination thereof. The elastic element or piece can be arranged in parallel with the needle base or seat, and/or allow the needle base or seat to pass through. For example, an elongated needle base or seat can pass through the coils of a spring, where a proximal end of the spring engages a proximal portion of the elongated needle base or seat, and a distal end of the spring engages a proximal portion of the piston rod. A distal portion of the elongated needle base or seat may be inserted in an internal lumen of the piston rod, and all or a portion of the syringe needle can be housed in the internal lumen of the piston rod. In some embodiments, prior to medical penetration using the syringe needle, the syringe needle is positioned in the internal lumen without pass through the distal end of the piston rod or the a seal (e.g., plunger seal 3 in FIG. 12) attached thereto. As such, in some embodiments, the pressing shaft (e.g., comprising or connected to elongated needle base or seat) can be configured to elastically engage the piston rod (e.g., via spring 5 in FIG. 12), a distal portion of the piston rod engaging a seal such that the seal can be configured as a floating seal.

In some embodiments, the piston rod (e.g., push rod 15 in FIG. 12) is configured to receive and/or house the syringe needle (e.g., syringe needle 6 in FIG. 12) or at least a portion thereof. In some embodiments, the piston rod is hollow. In some embodiments, the piston rod comprises an internal lumen configured to receive and/or house the syringe needle or at least a portion thereof. The internal lumen of the piston rod may but does not need to be configured to receive and/or house a flowable composition (e.g., a drug composition). In some embodiments, the internal lumen of the piston rod contains a gas (e.g., air) and houses the syringe needle but does not contain a liquid such as a drug solution. In some embodiments, the piston rod can be used to draw a flowable composition. In some embodiments, the piston rod can be pulled by a handle (e.g., handle 21 in FIG. 12) to draw the flowable composition into a syringe (e.g., syringe 1 in FIG. 12). In some embodiments, the piston rod can be used to inject a flowable composition. In some embodiments, the piston rod can be pushed by the pressing shaft (e.g., via spring 5 in FIG. 12), and the syringe needle inside the piston rod can pass through a seal (e.g., plunger seal 3 in FIG. 12) at the distal end of the piston rod. In some embodiments, there is a needle body opening between the proximal end and the distal end of the syringe needle, and the flowable composition in the syringe can contact the needle body opening once the needle body opening is distal to the seal. In some embodiments, a needle body passageway connects the needle body opening to a needle distal opening, such that a pressure different between the needle body opening (e.g., when it is inside the syringe and contacting the flowable composition) and the needle distal opening can drive the flowable composition through the needle body passageway, thereby injecting the flowable composition (through the needle distal opening) into an apparent or potential tissue void, cavity, or vessel.

In some embodiments, a portion of the piston rod (e.g., push rod 15 in FIG. 12) is configured to engage a guide tube (e.g., guide tube 16 in FIG. 12). In some embodiments, the guide tube is a tube provided inside the housing. In some embodiments, the guide tube is provided inside a another tube within the housing. In some embodiments, a portion of the piston rod slidably engages an inner surface of the guide tube, such that the piston rod can move axially relative to the guide tube. In some embodiments, a portion of the pressing shaft is configured to engage the guide tube. In some embodiments, a portion of the pressing shaft slidably engages an inner surface of the guide tube, such that the pressing shaft can move axially to move the syringe needle relative to the guide tube. In some embodiments, the guide tube may comprise a structure (e.g., one or more axial ridges or grooves) on an internal surface that slidably engage a corresponding structure (e.g., one or more axial grooves or ridges) on an outside surface of the pressing shaft and/or on the piston rod. The corresponding structures (e.g., axial ridges and grooves) can allow sliding movement of the pressing shaft and/or the piston rod in a axial direction, while maintaining positional stability and/or minimizing movement of the syringe needle in other directions (e.g., radially). In some embodiments, a proximal portion of the piston rod comprises a protrusion (e.g., one or more annular ridges) that engages an internal surface of the guide tube. As such, in some embodiments, the piston rod can be considered a floating structure in that it slidably engages an inner surface of the guide tube and can be moved axially relative to the guide tube due to the engagement of the piston rod with the spring. In some embodiments, the guide tube is fixed relative to the housing.

In some embodiments, a distal portion of the piston rod (e.g., push rod 15 in FIG. 12) is configured to engage the seal (e.g., plunger seal 3 in FIG. 12). In some embodiments, the piston rod is configured to slidably engage an internal surface of the syringe (e.g., syringe 1 in FIG. 12). In some embodiments, the seal is a floating seal that slidably and sealingly engages an internal surface of syringe. In some embodiments, the seal separates a proximal lumen and a distal lumen formed by a syringe barrel of the syringe, where the distal lumen of the syringe is configured to draw and/or store a flowable composition. In some embodiments, the seal together with a syringe barrel of the syringe forms a lumen that is configured to draw and/or store a flowable composition. In some embodiments, the seal is on the distal end of the piston rod inserted in the syringe.

In some embodiments, the syringe (e.g., syringe 1 in FIG. 12) is configured to engage the housing. In some embodiments, a proximal portion of the syringe fixedly or removably engages the housing. In some embodiments, a distal portion of the syringe fixedly or removably engages the distal seal (e.g., sealing tip 8 in FIG. 12). In some embodiments, an internal lumen of the syringe is configured to draw and/or store a flowable composition such as a drug composition. In some embodiments, the internal lumen configured to contain the flowable composition is distal to the floating seal (e.g., plunger seal) and proximal to the distal seal (or sealing tip), and is formed by sealing engagement between the syringe and the floating seal and between the syringe and the distal seal. In some embodiments, the distal seal sealingly engages the distal end of the syringe. In some examples, the distal seal can be pressed onto the distal end of the syringe in order to form the sealing engagement. Exemplary configurations of the distal seal are shown in FIGS. 16A-16C and the distal seal can have a flat distal portion, a spherical distal portion, or a cone-shaped distal portion. The distal seal can have a distal portion having a flat distal surface, a convex surface, a spherical surface, a concave surface, or a distal surface of any other suitable shape.

In some examples, the distal seal can comprise a proximal portion that inserts into the syringe to form a sealing engagement with an internal surface of the syringe. In some examples, the portion of the distal seal and the internal surface of the syringe can comprise corresponding structures (e.g., protrusions such as threads and ridges, e.g., an annular ridge, and indentations such as grooves, e.g., annular groove) that engage each other. For instance, the portion of the distal seal can comprise a thread on an outside surface that engages a thread on internal surface of a distal portion of the syringe. In some examples, the distal seal can comprise a portion that engages an outside surface of the syringe. In some examples, the portion of the distal seal and the outside surface of the syringe can comprise corresponding structures (e.g., protrusions such as threads and ridges, and indentations such as grooves) that engage each other. For instance, the portion of the distal seal can comprise a thread on an internal surface that engages a thread on an external surface of a distal portion of the syringe.

In some examples, the distal seal can comprise a proximal portion that engages a gland (e.g., gland 23 shown in FIG. 12). In some examples, an internal surface of the gland and an outside surface of the syringe can comprise corresponding structures (e.g., protrusions such as threads and ridges, e.g., an annular ridge, and indentations such as grooves, e.g., annular groove) that engage each other, e.g., via threaded engagement. In some examples, the gland engages the distal seal at an annular groove 24 and presses the distal seal against the distal opening of the syringe barrel to form a sealing engagement.

In some embodiments, a device disclosed herein comprises a stopper, e.g., limiter 18 in FIG. 12. In some embodiments, the stopper can be used to limit the maximal length of an axial movement of the pressing shaft, e.g., in order to achieve precise injection. In some embodiments, the stopper can be used to limit a rotation and/or a radial movement of the pressing shaft, e.g., in order to prevent or minimize deviation of the pressing shaft (and the needle base and syringe needle coupled thereto) from a central axis of the assembled device. In some embodiments, the stopper can engage the guide tube. In some embodiments, the stopper can engage the fixedly or removably engage the proximal end of the guide tube. In some embodiments, the guide tube can be used to guide the movement of the pressing shaft and the piston rod, e.g., through corresponding structures on the components, in order to achieve precision of the axial movement of the pressing shaft and the piston rod, as well as precision of the syringe needle movement. In some embodiments, the device through a combination of features (e.g., the stopper and the guide tube) prevents or minimizes the rotation and/or deviation (e.g., from a central axis) of the pressing shaft, the piston rod, the needle base or seat, and/or the syringe needle, both during transportation and storage of the assemble device and during the use of the device for medical penetration.

In some embodiments, a device disclosed herein comprises an adapter, e.g., adapter 20 in FIG. 12. In some embodiments, the adapter can comprise a distal end comprising a plurality of distal petals and/or a proximal end comprising a plurality of proximal petals. In some embodiments, the adapter can comprise an adapter needle. In some embodiments, the adapter can be used to transfer a flowable composition from a container (e.g., a vial) to the syringe of the device disclosed herein. In some embodiments, the syringe can be inserted into the proximal end of the adapter. For instance, the distal seal (e.g., sealing tip 8 in FIG. 12) can be inserted inside the adapter, where the adapter needle contacts and passes through the distal seal to establish a fluid communication with an internal lumen of the syringe, where the internal lumen of the syringe is distal to the floating seal. The fluid communication can allow passage of a gas, a liquid, or a mixture thereof. In some embodiment, the adapter needle can be inserted into a container (e.g., a vial) containing a flowable composition (e.g., a drug solution), e.g., by using the adapter needle to penetrate a seal of the container, thereby establishing a fluid communication between the internal lumen of the syringe and the inside of the container. In some embodiments, a handle (e.g., handle 21 in FIG. 12) configured to engage a proximal portion of the piston rod can be used to push and/or pull the piston rod in an axial direction relative to the syringe. For example, the handle can be pulled proximally to draw the flowable composition from the container into the internal lumen of the syringe via the adapter needle. In another example, the handle can be pushed distally to expel gas and/or liquid via the adapter needle. For instance, the handle can be pulled proximally to draw a liquid (e.g., a drug solution) along with undesired gas (e.g., air) into the syringe, and then the assembly comprising the syringe and the adapter can be positioned with the adapter needle pointing upwards (e.g., vertically), such that the handle can be pushed distally (e.g., in an upward direction) to expel the undesired gas through the adapter needle, leaving the flowable composition in the syringe.

In some examples, the syringe of a device disclosed herein can be prefilled with a flowable material or composition. In some embodiments, the syringe (e.g., syringe 1 show in FIG. 12) can be provided in one or more parts. In some embodiments, a container (e.g., a syringe unit) can comprise a cylindrical wall sealingly engaging a fixed seal (which can be fixed to the container at a distal end of the container and can be passed through by the needle) and a floating seal (which can move inside the container and can be passed through by the needle), and the space enclosed by the cylindrical wall, the fixed seal and the floating seal can be prefilled with a flowable material or composition. In some embodiments, the device or system can comprise a first syringe unit and the container can be a second syringe unit configured to engage the distal end of the first syringe unit. The container (e.g., syringe unit) can be inserted into or attached to the body (e.g., to the first syringe unit) of the device prior to or after the flowable material or composition is filled into the container (e.g., syringe unit). In some embodiments, the floating seal in the container (e.g., syringe unit) may contact the distal end of the piston rod, thereby establishing an engagement between the piston rod and the floating seal that transmits a force from the spring to the floating seal. The fixed seal at the distal end of the container (e.g., syringe unit) may contact a contacting element at the distal end of the device, and the contacting element can be a distal seal of the syringe. In some embodiments, the fixed seal of the container (e.g., syringe unit) also serves as a distal seal of the syringe and/or as a contacting element. In some embodiments, the container (e.g., syringe unit) can be configured to at least partially insert into a syringe barrel. In some embodiments, the fixed seal sealingly engages the container (e.g., syringe unit) which in turn engages an inside wall of the syringe barrel. In some embodiments, the fixed seal sealingly engages both the container (e.g., syringe unit) and an inside wall of the syringe barrel. The engagement between the container (e.g., syringe unit) and the syringe barrel and the engagement between the fixed seal and a wall of the container can comprise any suitable engagement, such as via insertion, a threaded engagement, a non-threaded engagement, engagement secured by a clip, engagement secured by a gland, or any combination thereof.

In some embodiments, a device disclosed herein achieves precise control of the syringe needle as it advances through one or more tissues, and is particular useful for accessing an apparent or potential tissue void, cavity, or vessel, such as potential space between two adjacent tissues having different densities. In some embodiments, a device disclosed herein achieves precise access of an epidural space, while reducing or minimizing the risk of insufficient penetration and/or the risk of overshooting, e.g., the needle going too deep. In some examples, the axial movement of the syringe needle can be controlled and reach micron precision as it advances in the tissue. In some examples, the axial movement distance of the syringe needle in the tissue can be set to be within a length of between about 0 and about 4.0 mm, e.g., between about 0 and about 0.5 mm, between about 0 and about 1.0 mm, between about 0 and about 1.5 mm, between about 0 and about 2.0 mm, or between about 0 and about 2.5 mm. In some embodiments, a device disclosed herein comprises a syringe needle (e.g., 6 as shown in FIG. 12) of a size and configuration disclosed herein, e.g., the syringe needle having a bevel angle between about 0 degree and about 40 degrees, particularly between about 5 degrees and about 30 degrees, such as between about 15 degree and about 25 degrees. In some embodiments, the volume of the flowable composition to be delivered (e.g., via injection) using a device disclosed herein can be selected based on the conditions of a particular subject, and can be adjusted according to changes in the conditions. In some embodiments, the energy stored in the energy storage member (e.g., spring) is automatically released to advance the floating seal (e.g., via a piston rod 15 in FIG. 12), thereby discharging a volume of the flowable composition into an apparent or potential tissue void, cavity, or vessel. Given the combination of various features disclosed herein, the devices and methods disclosed herein can achieve precise, safe, and controllable delivery of agents into a tissue of a subject, such as an apparent or potential tissue void, cavity, or vessel.

In some embodiments, a device or system disclosed herein comprises a cannula (e.g., a microcannula), a microneedle, and an operation module (e.g., comprising a handle or knob configured to control the advancement or retraction of the microneedle and the advancement or retraction of the flexible cannula). In some embodiments, the cannula comprises a distal tip which may comprise a sharp tip, a stylet, a bevel, or a blunt tip. In some embodiments, the cannula comprises a flexible body. In some embodiments, the operation module is configured to control cannula placement and delivery to achieve minimally invasive operation. In some embodiments, the microneedle has a curved tip and is configured to be housed in the cannula. In some embodiments, the microneedle is configured to be advanced and/or retracted through an internal lumen of the cannula. In some embodiments, a proximal end of the cannula is configured to be engage a distal connector of the operation module. In some embodiments, the operation module comprises one or more elements configured to engage the microneedle in order to control movement of the microneedle inside an internal lumen of the cannula. In some embodiments, a distal connector of the operation module is configured to engage one or more syringes through one or more adapters. In some embodiments, each syringe is connected to an adapter that is connected to the operation module. In some embodiments, the one or more syringes can contain one or more compositions, such as a flowable material, a viscoelastic material, or an infusate, and delivery of the composition(s) through the microneedle can be controlled.

In some embodiments, the linear member such as a cannula is a thin, flexible hollow tube with a smooth round tip on the distal end, where the opposite, proximal end can have a hub (e.g., a plastic hub) that can be attached to a syringe. In some embodiments, the cannula comprises a sharp distal tip. In some embodiments, the cannula comprises a blunt distal tip. In some embodiments, the distal end of the cannula opens up a path between structures in tissue, thereby helping dissecting the structures while reducing tissue damage. In some embodiments, the cannula can comprise an opening at its distal end, e.g., at a blunt-tip of the cannula. In some embodiments, the cannula can comprise a side opening on a side wall of the cannula, whereas the distal end may or may not comprise an opening.

In some embodiments, the device or system comprises a hollow needle comprising a proximal end, wherein the needle is slidable relative to the cannula. In some embodiments, the device or system comprises an actuation member coupled to the proximal end of the hollow needle to translate the hollow needle. In some embodiments, the hollow needle is configured to translate relative to the cannula to thereby drive a distal portion of the needle along an exit axis that is obliquely oriented relative to the longitudinal axis of the cannula.

In some embodiments, the device or system comprises a fluid source in fluid communication with the proximal end of the hollow needle. In some embodiments, the needle includes a sharp distal tip. In some embodiments, the sharp distal tip of the needle comprises a first bevel, a second bevel, and optionally a third bevel, wherein the first bevel, second bevel, and optional third bevel are each oriented obliquely relative to each other. In some embodiments, the exit axis is oriented at an angle between approximately 5° and approximately 30° relative to the longitudinal axis of the cannula. In some embodiments, the exit axis is oriented at an angle between approximately 7° and approximately 9° relative to the longitudinal axis of the cannula. In some embodiments, the cannula includes a beveled distal end, wherein the beveled distal end has a bevel angle, wherein the bevel angle is between approximately 10° and approximately 30°. In some embodiments, the cannula defines a plurality of lumens extending longitudinally through the length of the cannula, wherein at least one lumen of the plurality of lumens is configured to slidably receive the needle. In some embodiments, the cannula has a flexural stiffness between 0.5×10⁻⁶ N/mm and 12×10⁻⁶ N/mm. In some embodiments, the cannula has a flexural stiffness between 2.0×10⁻⁶ N/mm and 8.0×10⁻⁶ N/mm. In some embodiments, the cannula has a bending stiffness of about 1.0×10⁻⁶, about 1.5×10⁻⁶, about 2.0×10⁻⁶, about 2.2×10⁻⁶, about 2.4×10⁻⁶, about 2.6×10⁻⁶, about 2.8×10⁻⁶, about 3.0×10⁻⁶, about 3.2×10⁻⁶, about 3.4×10⁻⁶, about 3.6×10⁻⁶, about 3.8×10⁻⁶, about 4.0×10⁻⁶, about 4.2×10⁻⁶, about 4.4×10⁻⁶, about 4.6×10⁻⁶, about 4.8×10⁻⁶, about 5.0×10⁻⁶, about 5.2×10⁻⁶, about 5.4×10⁻⁶, about 5.6×10⁻⁶, about 5.8×10⁻⁶, about 6.0×10⁻⁶, about 6.2×10⁻⁶, about 6.4×10⁻⁶, about 6.6×10⁻⁶, about 6.8×10⁻⁶, about 7.0×10⁻⁶, about 7.2×10⁻⁶, about 7.4×10⁻⁶, about 7.6×10⁻⁶, about 7.8×10⁻⁶, about 8.0×10⁻⁶, about 8.5×10⁻⁶, about 9.0×10⁻⁶, about 9.5×10⁻⁶, about 10.0×10⁻⁶, about 10.5×10⁻⁶, about 11.0×10⁻⁶, or about 11.5×10⁻⁶ N/mm.

In some embodiments, provided herein is a method for use of a device or system comprising a cannula and a hollow needle that is movable relative to the cannula.

The exemplary embodiments and optional implementations of the present disclosure are described in detail above in combination with the figures. However, the present disclosure is not limited to the details described in the embodiments described above. Simple variants can be applied to the embodiments of the present disclosure, all of which are within the scope of the present disclosure.

It should be appreciated that any suitable injection device or systems, including but not limited to those described herein in connection with the figures, may be used in a method for epidural injection disclosed herein, or combined with any of the catheter guiding structures described herein for placing a catheter into the epidural space. For instance, an injection device or system shown in FIG. 12A may be used. In some embodiments, the injection device or system comprises a syringe barrel comprising a proximal end and a distal end; a floating seal in the syringe barrel; a puncture member such as a needle at the distal end of the syringe barrel, wherein the puncture member is not attached to the floating seal; and an actuation member configured to elastically engage the floating seal via an energy storage member such as a spring or the like and/or another suitable elastic member. In some embodiments, the puncture member comprises a distal end opening configured to form a fluidic communication with a lumen in the syringe barrel containing a flowable composition. In some embodiments, the injection device or system further comprises a stopper in the syringe barrel, between the floating seal and the distal end of the syringe barrel. As shown in FIG. 12A, Step 1, the injection device or system is in an initial state where the distal end opening of the puncture member has not entered a tissue of a subject, and the distance between the actuation member and the floating seal is x₁. In FIG. 12A, Step 2, the distal end opening of the puncture member has entered a relatively dense tissue, where the distance between the actuation member and the floating seal remains the same (x₁). In FIG. 12A, Step 3, the distal end opening of the puncture member remains in the relatively dense tissue (e.g., ligament tissue), when the energy storage member is compressed by reducing the distance between the actuation member and the floating seal from x₁ to x₂. This way, the energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 12A, Step 4, the puncture member is advanced distally into a less dense tissue, such as an apparent or potential tissue void, cavity, or vessel (for instance, the epidural space). In some embodiments, due to the decrease in tissue density, the back pressure on the distal opening of the puncture member becomes less than the pressure of the flowable composition, thereby allowing release of the flowable composition into the less dense tissue, such as the apparent or potential tissue void, cavity, or vessel. As the flowable composition is discharged from the distal end opening of the puncture member, energy in the energy storage member is released, thereby increasing the distance between the actuation member and the floating seal from x₂ to x₃, as shown in FIG. 12A, Step 5. Distal movement of the floating seal in the syringe barrel may be stopped by the stopper, for example, in order to control the volume of the flowable composition delivered into the less dense tissue.

Another example is shown in FIG. 12B, Step 1, where the medical puncture device is in an initial state where the distal end opening of the puncture member has not entered a tissue of a subject, and in FIG. 12B, Step 2, the energy storage member can be compressed, whereas the distal end opening of the puncture member remains outside a tissue and the floating seal is not advanced distally to discharge the flowable composition from the distal end opening. In FIG. 12B, Step 3, the distal end opening of the puncture member has entered a relatively dense tissue (e.g., the ligament tissue). The energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 12B, Step 4, the distal end opening of the puncture member starts to enter a less dense tissue, such as an apparent or potential tissue void, cavity, or vessel (for instance, the epidural space), whereas the energy storage member remains compressed. In FIG. 12B, Step 5, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled. The force applied onto the actuation member may be released as shown in FIG. 12B, Step 6.

Yet another example is shown in FIG. 12C. In some embodiments, the injection device or system comprises a syringe barrel comprising a proximal end and a distal end; a floating seal in the syringe barrel; a puncture member such as a needle at the distal end of the syringe barrel, wherein the puncture member is not attached to the floating seal; and an energy storage member configured to elastically engage the floating seal and the proximal end of the syringe barrel. In some embodiments, the injection device or system further comprises a stopper in the syringe barrel, between the floating seal and the distal end of the syringe barrel. In some embodiments, the medical puncture device comprises a contact member. In FIG. 12C, Step 1, the medical puncture device is in an initial state where the distal end opening of the puncture member is in the contact member which prevents discharge of the flowable composition from the distal end opening. The energy storage member applies a force onto the floating seal, and through the flowable composition and the distal opening of the puncture member, a pressure is in turn applied to the contact member. Due to the density of the contact member, the back pressure on the distal opening of the puncture member prevents leakage of the flowable composition from the syringe barrel. In FIG. 12C, Step 2, the distal end opening of the puncture member has entered a relatively dense tissue (e.g., the ligament tissue), and the back pressure of the relatively dense tissue on the distal opening prevents leakage of the flowable composition into the tissue. In FIG. 12C, Step 3, the distal end opening of the puncture member starts to enter a less dense tissue, such as an apparent or potential tissue void, cavity, or vessel (for instance, the epidural space). In FIG. 12C, Step 4, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled.

FIG. 13 shows for epidural anesthesia, an epidural needle (e.g., a styletted needle) can be inserted through the skin and subcutaneous tissue, the supraspinous and interspinous ligaments, and into the ligamentum flavum. As the needle is advanced, the epidural space is identified by loss of resistance. The syringe can then be removed, and an epidural catheter can be advanced through the needle and into the epidural space. The needle can be removed over the catheter, leaving a portion (e.g., 4 to 6 cm) of the catheter in the epidural space.

In some embodiments, the injection system or device provided in FIGS. 14A-14B and FIG. 15 is used for a method of epidural injection, wherein the injection system or device comprises: a syringe barrel 1 extending from a proximal end to a distal end; a first hollow needle 6 extending from a proximal end to a distal end comprising an end opening, wherein the needle distal end is connected to the distal end of the syringe barrel; a floating seal 3, wherein the floating seal is positioned inside the syringe barrel, forms a lumen between the floating seal and the distal end of the syringe barrel, and comprises a hollow channel aligned with the first hollow needle; a push shaft 2 with a hollow channel extending from a proximal end to a distal end, wherein the distal end of the push shaft is proximal to and in contact with the floating seal, and the hollow channel of the push shaft is aligned with the hollow channel of the floating seal and the first hollow needle to form a central hollow channel extending from the proximal end of the push shaft to the distal opening of the first hollow needle; a proximal seal 8a at the proximal end of the central hollow channel; and an actuation unit comprising an actuation member 2a (e.g., a control knob) and an energy storage member 5 (e.g., a spring), wherein the actuation member 2a can elastically engage with the push shaft 2 via the energy storage member 5. In some embodiments, the injection system or device further comprises a piercing unit 6′, which can pierce the proximal seal 8a and open the central hollow channel. In some embodiments, the injection device or system further comprises a needle guiding structure 43 with a hollow channel. In some embodiments, the injection device or system further comprises a catheter guiding channel 12b, from which a catheter 11 can be inserted into the central hollow channel, and ultimately into the epidural space.

In some embodiments, the operation of an injection system or device, for example, as shown in FIGS. 14A-14B, for injecting a flowable composition and/or placing an implant (e.g., catheter) into the epidural space, is shown in FIG. 15. In FIG. 15, Step 1, the injection system or device is in an initial state where the distal end opening of the first hollow needle 6 has not entered a tissue of a subject, and wherein the energy storage member 5 is at its static state. In FIG. 15, Step 2, the distal end opening of the first hollow needle 6 has reached a dense tissue (e.g., the ligamentum flavum), and the energy storage member 5 (e.g., spring) is compressed by dialing the control knob 2a, wherein the pressure from the dense tissue (e.g., the ligamentum flavum) balances the pressing force from the energy storage member 5, so floating seal 3 is not advanced distally. In FIG. 15, Step 3, the distal end opening of the first hollow needle 6 has reached a less dense tissue (e.g., the epidural space). In some embodiments, the pressure at the distal end opening of the first hollow needle 6 drops and becomes less than the pressing pressure from the energy storage member 5, therefore allowing the distal movement of the floating seal 3 without any manual movement of the push shaft. This automatic movement of the floating seal 3 provides a clear visual indication that the distal end opening of the first hollow needle 6 has reached the epidural space, when a flowable material can be injected and/or an implant (e.g., catheter) can be installed. In FIG. 15, Step 4, the distal end opening of the first hollow needle 6 has reached the epidural space, and the piercing unit 6′ is advanced distally to pierce the proximal seal 8a, so that a hollow channel is formed from the proximal end of a needle guiding structure 43 all the way to the distal end opening of the first hollow needle 6.

In some embodiments, the guiding structure 43 can comprise a side port 44, such that when the guiding structure 43 is advanced distally, the side port aligns with a catheter guiding structure with a hollow channel (e.g., 12b in FIG. 14B and FIG. 15), and a catheter can be inserted through the hollow channel of the catheter guiding structure, through the side port, and into the hollow needle guiding channel inside the guiding structure 43 and further inserted into the central hollow channel inside push shaft 2, into the hollow puncture needle 6, and eventually into an epidural space.

In FIG. 15, Step 5, a second syringe 50 with a hollow needle is inserted sequentially through the needle guiding structure 43, a hollow channel within the piercing unit 6′, the pierced proximal seal 8a, the hollow channel in push shaft 2, the hollow channel in the floating seal 3, and the first hollow needle 6, and ultimately punctures into the epidural space through the distal end opening of the first hollow needle 6. A flowable material (e.g., a drug composition) contained in the second syringe 50 can then be injected into the epidural space via the needle of the second syringe. In some embodiments, the injection of a flowable materials is necessary or desirable, therefore a step such as Step 5 in FIG. 15 is performed. In some embodiments, the injection of a flowable material is not necessary or desirable, therefore a step such as Step 5 in FIG. 15 is omitted. In some embodiments, as shown in FIG. 15, Step 6, after piercing the proximal seal 8a, and optionally after injecting a flowable material via a second syringe 50, a catheter 11 is advanced into the central hollow channel and ultimately placed into the epidural space via the distal end opening of the first hollow needle 6. In some embodiments, the distal advancement of the catheter 11 is controlled by a catheter insertion unit 42. In some embodiments, the catheter insertion unit is a pair of gears wherein the catheter 11 is located in between the two gears. In some embodiments, the distal advancement of the catheter 11 is achieved by dialing the gears. In some embodiments, the distal end of the catheter 11 sequentially pass through a catheter stabilizing structure 12a, a catheter guiding channel 12b, part of the needle guiding structure 43, the pierced proximal seal 8a, the hollow channel of the push shaft 2, the hollow channel of the floating seal 3, and the first hollow needle 6. In this way, the distal end of the catheter is placed into the epidural space, from which drugs can be delivered to the epidural space continuously if needed. Once the catheter has been inserted at the target site, the injection system or device can be removed, leaving the inserted catheter at the target site.

In some embodiments, the operation of an injection system or device, for example, as shown in FIGS. 14A-14B, for injecting a flowable composition and/or placing an implant (e.g., catheter) into the epidural space, is shown in FIG. 15.

In some embodiments, the operation of an integrated device, for example, as shown in FIGS. 16A-16B, or 17, for injecting a flowable composition and/or placing an implant (e.g., catheter) into the epidural space, is shown in FIG. 18. In FIG. 18, Step 1, the integrated device is in an initial state by coupling a needle (e.g., 5 in FIG. 17) to a device, wherein the needle comprises a needle tip (e.g., 18 in FIG. 17), a needle lumen (e.g., 6 in FIG. 17) and a needle hub (e.g., 14 in FIG. 17). In some embodiments, the integrated device comprises: i) a syringe barrel (e.g., 1 in FIG. 17) extending from a proximal end to a distal end, wherein the distal end of the syringe barrel comprises a needle base (e.g., 4 in FIG. 17) configured to be coupled to the needle hub, and wherein the needle base comprises a passageway (e.g., 7 in FIG. 17) configured to fluidically communicate with the needle lumen; ii) a gasket seal (e.g., 3 in FIG. 17) that forms a fluid-tight seal with an inner wall of the syringe barrel, wherein the gasket seal abuts the distal end of the syringe barrel, wherein the gasket seal comprises a through hole (e.g., 9 in FIG. 17) along an axis of the syringe barrel, and wherein the through hole aligns with the passageway in the needle base; iii) a push shaft (e.g., 2 in FIG. 17) extending from a proximal end to a distal end, wherein the distal end of the push shaft engages the gasket seal, and the push shaft comprises: a central channel (e.g., 10 in FIG. 17) comprising a distal end that aligns with the through hole of the gasket seal; and a valve (e.g., 11 in FIG. 17) that aligns with a proximal end of the central channel; iv) a spring (e.g., 12 in FIG. 17), wherein a distal end of the spring engages a portion of the push shaft, and a proximal end of the spring engages a structure (e.g., a baffle) in or of the syringe barrel, wherein the spring is configured to be compressed; and v) a catheter (e.g., 16 in FIG. 17) configured to be inserted through the valve and into the central channel. In this initial state, the distal tip (e.g., 18 in FIG. 17) of the needle (e.g., 5 in FIG. 17) has not entered a tissue of a subject, and wherein the elastic element (e.g. the spring, 12 in FIG. 17) is at its static state.

In FIG. 18, Step 2, the push shaft is proximally actuated, thereby compressing the spring and forming a flowable composition lumen (e.g., 8 in FIG. 17) between the gasket seal and the distal end of the syringe barrel, wherein the flowable composition lumen contains a gas. In FIG. 18, Step 2, the push shaft is locked to maintain a compressed state of the spring and maintain a position of the gasket seal in the syringe barrel.

In FIG. 18, Step 3, the needle tip is advance in the subject towards a location in the ligamenta flava of the subject, without a user holding the push shaft to maintain the compressed state of the spring. In FIG. 18, Step 3, the push shaft is unlocked when the needle tip is in the ligamenta flava. Once the distal tip (e.g., 18 in FIG. 17) of the needle has reached the ligamenta flava (or other dense tissue) of the subject, the pressure from the dense tissue balances the pressing force from the compressed state of the spring, so the push shaft or the gasket seal is not advanced distally.

In FIG. 18, Step 4, the needle tip is advanced through the ligamenta flava into an epidural space of the subject, thereby allowing the compressed spring to decompress such that the gasket seal is moved distally to connect the central channel and the passageway in the needle base. Once the distal tip (e.g., 18 in FIG. 17) of the needle has reached the epidural space (or a less dense tissue), the pressure at the distal tip drops and becomes less than the pressing pressure from the compressed state of the spring, therefore allowing the distal movement of the push shaft or the gasket seal without any manual movement of the push shaft. This automatic movement of the push shaft or the gasket seal provides a clear visual indication that the distal tip of the needle has reached the epidural space, when a flowable material can be injected and/or an implant (e.g., catheter) can be installed.

In FIG. 18, Step 5, an anesthetic agent is injected through a side port of the needle into the needle lumen, thereby injecting anesthetic agent into the epidural space of the subject. In some embodiments, the anesthetic agent is delivered by a second syringe through the side port of the needle into the needle lumen.

Optionally in FIG. 18, Step 6, the second syringe for injecting the anesthetic agent is uncoupled from the side port of the needle, thereby emptying the needle lumen for subsequent insertion of the catheter from the integrated device. A hollow channel is formed from the proximal tip (e.g., 18 in FIG. 17) of the needle through the needle lumen (e.g., 6 in FIG. 17), the passageway (e.g., 7 in FIG. 17) of the needle base, the through hole (e.g., 9 in FIG. 17) of the gasket seal, and the central channel (e.g., 10 in FIG. 17), all the way to the valve.

In FIG. 18, Step 7, the catheter is inserted through the valve, the central channel, the through hole, the passageway, and the needle lumen, thereby placing a distal portion of the catheter in the epidural space. In some embodiments, the integrated device comprise a catheter guiding channel (e.g., 13 in FIG. 17). In some embodiments, the catheter guiding channel further comprises a side port (e.g., 19 in FIG. 17), wherein the side port is configured to align with the catheter guiding channel. In some embodiments, the catheter is inserted sequentially through a catheter stabilizing structure (e.g., 17 in FIG. 17), the side port, the catheter guiding channel, the valve, the central channel, the passageway of the gasket seal, the passageway of the needle base, the needle lumen, the distal tip of the needle, and eventually into an epidural space. In some embodiments, the distal advancement of the catheter (e.g., 16 in FIG. 17) is controlled by a catheter insertion unit (e.g., 42 in FIG. 17). In some embodiments, the catheter insertion unit is a pair of gears wherein the catheter is located in between the two gears. In some embodiments, the distal advancement of the catheter is achieved by dialing the gears.

In FIG. 18, Step 7, the needle is uncoupled from the needle base to remove the catheter from the device, while the distal portion of the catheter remains in the epidural space. In this way, the distal end of the catheter is placed into the epidural space, from which drugs can be delivered to the epidural space continuously if needed.

In some embodiments, the catheter is used for epidural anesthesia by continuous infusion or intermittent bolus, optionally by manually delivered intermittent bolus (MIB) and programmable intermittent bolus (PIB). In some embodiments, the catheter is used for intraoperative epidural anesthesia and/or postoperative analgesia.

It should be noted that, each of the technical features described in the embodiments above, when not in conflict, can be combined in any reasonable manner. To avoid unnecessary repetition, the possible combinations are not described separately in the embodiments.

Additionally, the different implementations of the embodiments of the present disclosure can be freely combined. As long as they do not go against the ideas of the present disclosure, they should also be considered part of this disclosure.

1. A device, comprising:
a) a syringe barrel extending from a proximal end to a distal end, wherein the distal end of the syringe barrel comprises a needle base configured to be coupled to a needle comprising a needle lumen, and wherein the needle base comprises a passageway configured to fluidically communicate with the needle lumen;
b) a gasket seal that forms a fluid-tight seal with an inner wall of the syringe barrel, wherein a flowable composition lumen is formed between the gasket seal and the distal end of the syringe barrel, wherein the gasket seal comprises a through hole, and wherein the through hole is configured to align with the passageway in the needle base;
c) a push shaft extending from a proximal end to a distal end, wherein the distal end of the push shaft engages the gasket seal, and the push shaft comprises:
a central channel comprising a distal end that aligns with the through hole of the gasket seal; and
a valve that aligns with a proximal end of the central channel;
d) an elastic element configured to actuate the push shaft such that the gasket seal is moved distally along the axis of the syringe barrel; and
e) a catheter configured to be inserted through the valve and into the central channel.

2. The device of claim 1, wherein the needle is an epidural needle.

3. (canceled)

4. The device of claim 1, wherein the needle is between about 17 G and about 22 G (iso-9626) in gauge size.

5-7. (canceled)

8. The device of claim 1, wherein the needle comprises a straight or beveled distal tip.

9. (canceled)

10. (canceled)

11. The device of claim 1, wherein the needle comprises a side port.

12. (canceled)

13. The device of claim 1, wherein the gasket seal is configured to allow distal advancement of the catheter through the through hole, optionally wherein the through hole is along the axis of the syringe barrel.

14. The device of claim 1, wherein the gasket seal is configured to allow or prevent proximal retraction of the catheter through the through hole.

15. The device of claim 1, wherein the through hole is configured to close or remain open in the absence of the catheter or the central channel inserted in the gasket seal.

16. The device of claim 1, wherein the through hole is configured to allow the catheter or the central channel to pass through the gasket seal.

17. The device of claim 1, wherein a distal end of the central channel passes through or abuts the through hole of the gasket seal.

18. The device of claim 1, wherein a distal end of the central channel extends distally beyond the gasket seal, or wherein a distal end of the central channel is flush with the distal end of the through hole, or wherein a distal end of the central channel is flush with the proximal end of the through hole.

19. (canceled)

20. The device of claim 1, wherein the push shaft further comprises a central chamber in fluidic communication with the central channel, and wherein the central chamber is at least partially between the valve and the proximal end of the central channel.

21. (canceled)

22. The device of claim 1, wherein the push shaft comprises a catheter guiding channel, and wherein the valve aligns with a distal end of the catheter guiding channel.

23. (canceled)

24. (canceled)

25. The device of claim 1, wherein the push shaft comprises a locking mechanism configured to maintain a position of the gasket seal in the syringe barrel and a compressed or expanded state of the elastic element.

26. (canceled)

27. The device of claim 1, wherein a distal end of the elastic element engages a portion of the push shaft or the gasket seal, and a proximal end of the elastic element engages a portion of the syringe barrel.

28-39. (canceled)

40. A device, comprising:
a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen;
b) a gasket seal that forms a fluid-tight seal with an inner wall of the syringe barrel, wherein the gasket seal abuts the distal end of the syringe barrel, wherein the gasket seal comprises a through hole, and wherein the through hole aligns with the passageway in the needle base;
c) a push shaft extending from a proximal end to a distal end, wherein the distal end of the push shaft engages the gasket seal, and the push shaft comprises:
a central channel comprising a distal end that aligns with the through hole of the gasket seal; and
a valve that aligns with a proximal end of the central channel;
d) a spring, wherein a distal end of the spring engages a portion of the push shaft, and a proximal end of the spring engages a structure (e.g., a baffle) in or of the syringe barrel, wherein the spring is configured to be compressed, and decompression of the compressed spring actuates the push shaft or the gasket seal such that the gasket seal is moved distally along the axis of the syringe barrel;
e) a catheter configured to be inserted through the valve and into the central channel, and
f) a housing accommodating at least a portion of the syringe barrel, at least a portion of the push shaft, and at least a portion of the catheter.

41. A device, comprising:
a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen;
b) a gasket seal that forms a fluid-tight seal with an inner wall of the syringe barrel, wherein a flowable composition lumen is formed between the gasket seal and the distal end of the syringe barrel, wherein the gasket seal comprises a through hole, and wherein the through hole aligns with the passageway in the needle base;
c) a push shaft extending from a proximal end to a distal end, wherein the distal end of the push shaft engages the gasket seal, and the push shaft comprises:
a central channel comprising a distal end that aligns with the through hole of the gasket seal; and
a valve that aligns with a proximal end of the central channel;
d) a spring, wherein a distal end of the spring engages a portion of the push shaft, and a proximal end of the spring engages a structure (e.g., a baffle) in or of the syringe barrel, wherein the spring is compressed, and decompression of the compressed spring actuates the push shaft or the gasket seal such that the gasket seal is moved distally along the axis of the syringe barrel;
e) a catheter configured to be inserted through the valve and into the central channel, and
f) a housing accommodating at least a portion of the syringe barrel, at least a portion of the push shaft, and at least a portion of the catheter.

42. (canceled)

43. (canceled)

44. A device, comprising:
a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen;
b) a gasket seal that forms a fluid-tight seal with an inner wall of the syringe barrel, wherein the gasket seal comprises a through hole, and wherein the through hole aligns with the passageway in the needle base;
c) a push shaft extending from a proximal end to a distal end, wherein the distal end of the push shaft engages the gasket seal, and the push shaft comprises:
a central channel comprising a distal end that aligns with the through hole of the gasket seal; and
a valve that aligns with a proximal end of the central channel;
d) a spring, wherein a distal end of the spring engages a portion of the push shaft, and a proximal end of the spring engages a structure (e.g., a baffle) in or of the syringe barrel, wherein the spring exerts a force on the push shaft or the gasket seal such that the gasket seal abuts the distal end of the syringe barrel, and wherein the central channel and the passageway in the needle base are configured to be connected;
e) a catheter configured to be inserted through the valve and into the central channel, and
f) a housing accommodating at least a portion of the syringe barrel, at least a portion of the push shaft, and at least a portion of the catheter.

45. A device, comprising:
a) a syringe barrel extending from a proximal end to a distal end, wherein the distal end of the syringe barrel comprises a needle base coupled to a needle comprising a needle lumen, and wherein the needle base comprises a passageway fluidically communicating with the needle lumen;
b) a gasket seal that forms a fluid-tight seal with an inner wall of the syringe barrel, wherein the gasket seal comprises a through hole, and wherein the through hole aligns with the passageway in the needle base;
c) a push shaft extending from a proximal end to a distal end, wherein the distal end of the push shaft engages the gasket seal, and the push shaft comprises:
a central channel comprising a distal end that aligns with the through hole of the gasket seal;
a valve that aligns with a proximal end of the central channel; and
a catheter guiding channel, wherein the valve aligns with a distal end of the catheter guiding channel;
d) a spring, wherein a distal end of the spring engages a portion of the push shaft, and a proximal end of the spring engages a structure (e.g., a baffle) in or of the syringe barrel, wherein the spring exerts a force on the push shaft or gasket seal such that the gasket seal abuts the distal end of the syringe barrel, and wherein the central channel and the passageway in the needle base are connected;
e) a catheter, wherein a portion of the catheter is in the catheter guiding channel; and
f) a housing accommodating at least a portion of the syringe barrel, at least a portion of the push shaft, and at least a portion of the catheter.

46-50. (canceled)

51. A method for accessing an epidural space of a subject, comprising:
a) coupling a needle to a device, wherein the needle comprises a needle tip, a needle lumen, and a needle hub, and the device comprises:
i) a syringe barrel extending from a proximal end to a distal end, wherein the distal end of the syringe barrel comprises a needle base configured to be coupled to the needle, and wherein the needle base comprises a passageway configured to fluidically communicate with the needle lumen;
ii) a gasket seal that forms a fluid-tight seal with an inner wall of the syringe barrel, wherein the gasket seal abuts the distal end of the syringe barrel, wherein the gasket seal comprises a through hole along an axis of the syringe barrel, and wherein the through hole aligns with the passageway in the needle base;
iii) a push shaft extending from a proximal end to a distal end, wherein the distal end of the push shaft engages the gasket seal, and the push shaft comprises:
a central channel comprising a distal end that aligns with the through hole of the gasket seal; and
a valve that aligns with a proximal end of the central channel;
iv) a spring, wherein a distal end of the spring engages a portion of the push shaft, and a proximal end of the spring engages a structure (e.g., a baffle) in or of the syringe barrel, wherein the spring is configured to be compressed; and
v) a catheter configured to be inserted through the valve and into the central channel; and
b) proximally actuating the push shaft, thereby compressing the spring and forming a flowable composition lumen between the gasket seal and the distal end of the syringe barrel, wherein the flowable composition lumen contains a gas.

52-60. (canceled)
